(19) United States
(12) Patent Application Publication
(10) Pub. No.: US 2012/0135975 A1
(43) Pub. Date: May 31, 2012

(54) Substituted Esters as Cannabinoid-1 Receptor Modulators

(75) Inventors: Vincent J. Colandrea, North Brunswick, NJ (US); William K. Hagmann, Westfield, NJ (US); Jeffrey J. Hale, Westfield, NJ (US); Irene E. Whitney, New Haven, CT (US)

(73) Assignee: MERCK & CO., INC., rAHWAY, NJ (US)

(21) Appl. No.: 12/227,035

(22) PCT Filed: May 14, 2007
(86) PCT No.: PCT/US07/11548

§ 371 Date: Nov. 5, 2008

Related U.S. Application Data
(60) Provisional application No. 60/801,291, filed on May 18, 2006.

(51) Int. Cl. A61K 31/397; C07D 213/80; C07D 213/79; C07D 285/06; C07D 231/14; C07D 241/24; C07D 487/04; A61K 31/277; A61K 31/455; A61K 31/4402; A61K 31/4409; A61K 31/433; A61K 31/415; A61K 31/4965; A61K 31/42; A61K 31/519; A61K 31/4985; A61P 25/18; A61P 25/28; A61P 25/06; A61P 25/00; A61P 29/00; A61P 25/22; A61P 25/08; A61P 25/16; A61P 25/30; A61P 1/00; A61P 1/16; A61P 11/06; A61P 3/04; A61P 25/32; A61P 25/34; A61P 25/36; A61K 31/366; C07C 255/60

(52) U.S. Cl. 514/210.02; 558/414; 546/322; 546/326; 548/127; 548/374.1; 544/406; 544/281; 514/522; 514/356; 514/357; 558/416; 514/361; 514/406; 514/255.06; 514/378; 514/259.3; 514/249; 514/460

(57) ABSTRACT

Novel compounds of the structural formula (I) are antagonists and/or inverse agonists of the Cannabinoid-1 (CB1) receptor and are useful in the treatment, prevention and suppression of diseases mediated by the CB1 receptor. The compounds of the present invention are useful as centrally acting drugs in the treatment of psychosis, memory deficits, cognitive disorders, Alzheimer's disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, the treatment of obesity or eating disorders, as well as the treatment of asthma, constipation, chronic intestinal pseudo-obstruction, cirrhosis of the liver, non-alcoholic fatty liver disease (NAFLD), non-alcoholic steatohepatitis (NASH), and the promotion of wakefulness.

### BACKGROUND OF THE INVENTION

Marijuana (Cannabis sativa L.) and its derivatives have been used for centuries for medicinal and recreational purposes. A major active ingredient in marijuana and hashish has been determined to be Δ⁹-tetrahydrocannabinol (Δ⁹-THC). Detailed research has revealed that the biological action of Δ⁹-THC and other members of the cannabinoid family occurs through two G-protein coupled receptors termed CB1 and CB2. The CB1 receptor is primarily found in the central and peripheral nervous systems and to a lesser extent in several peripheral organs. The CB2 receptor is found primarily in lymphoid tissues and cells. Three endogenous ligands for the cannabinoid receptors derived from arachidonic acid have been identified (anandamide, 2-arachidonoyl glycerol, and 2-arachidonyl glycerol ether). Each is an agonist with activities similar to Δ⁹-THC, including sedation, hypothermia, intestinal immobility, antinociception, analgesia, catalepsy, anti-emesis, and appetite stimulation.

There are at least two CB1 modulators characterized as inverse agonists/antagonists, ACOMPLIA (rimonabant, N-(1-piperidinyl)-5-(4-chlorophenyl)-1-(2,4-dichlorophenyl)-4-methylpyrazole-3-carboxamide, SR141716A), and 3-(4-chlorophenyl-N′-(4-chlorophenyl)sulfonyl-N-methyl-4-phenyl-4,5-dihydro-1H-pyrazole-1-carboxamide (SLV-319), in clinical trials for treatment of eating disorders and/or smoking cessation at this time. There still remains a need for potent low molecular weight CB1 modulators that have pharmacokinetic and pharmacodynamic properties suitable for use as human pharmaceuticals.

WO 03/077847, 03/082190, 03/086288, 03/087037, 04/048317, 04/058145, 05/009479, 05/027837, 05/044785 describe CB1 receptor antagonists/inverse agonists with an acyclic core.

### SUMMARY OF THE INVENTION

The present invention is concerned with novel substituted esters of structural Formula I:

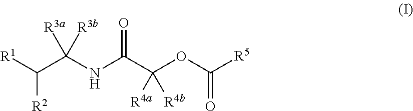

and pharmaceutically acceptable salts thereof which are modulators of and, in particular, antagonists and/or inverse agonists of the Cannabinoid-1 (CB1) receptor and are useful in the treatment, prevention or suppression of diseases mediated by the Cannabinoid-1 (CB1) receptor. In one aspect, the invention is concerned with the use of these novel compounds to selectively antagonize the Cannabinoid-1 (CB1) receptor. As such, compounds of the present invention are useful as centrally acting drugs in the treatment of psychosis, memory deficits, cognitive disorders, Alzheimer's disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, particularly abuse and/or addiction to opiates, alcohol, marijuana, and nicotine, including smoking cessation. The compounds are also useful for the treatment of obesity or eating disorders associated with excessive food intake and complications associated therewith, including left ventricular hypertrophy. The compounds are also useful for the treatment of constipation and chronic intestinal pseudo-obstruction. The compounds are also useful for the treatment of cirrhosis of the liver, non-alcoholic fatty liver disease (NAFLD) and non-alcoholic steatohepatitis (NASH). The compounds are also useful for the treatment of asthma and promotion of wakefulness.

The present invention is also concerned with treatment of these conditions, and the use of compounds of the present invention for manufacture of a medicament useful in treating these conditions. The present invention is also concerned with treatment of these conditions through a combination of compounds of formula I and other currently available pharmaceuticals.

The invention is also concerned with pharmaceutical formulations comprising one of the compounds as an active ingredient.

The invention is further concerned with processes for preparing the compounds of this invention.

### DETAILED DESCRIPTION OF THE INVENTION

The compounds of the present invention are represented by the compound of structural formula I:

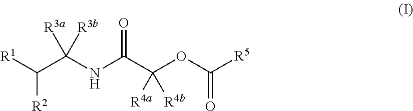

or a pharmaceutically acceptable salt thereof, wherein;

R¹ is selected from: C₁₋₁₀alkyl, C₃₋₁₀cycloalkyl, C₃₋₁₀cycloalkyl-C₁₋₄alkyl, cycloheteroalkyl, cycloheteroalkyl-C₁₋₄alkyl, aryl, aryl-C₁₋₄alkyl, heteroaryl, heteroaryl-C₁₋₄alkyl, —OR^{e}, —NR^{c}R^{d}, —NR^{c}C(O)R^{e}, —CO₂R^{e}, and —C(O)NR^{c}R^{d}, wherein each alkyl is optionally substituted with one to four substituents independently selected from R^{a}, and each cycloalkyl and cycloheteroalkyl are optionally substituted with one to four substituents selected from R^{b} and oxo, and each aryl and heteroaryl are optionally substituted with one to four substituents independently selected from R^{b};

R² is selected from: C₁₋₁₀alkyl, C₃₋₁₀cycloalkyl-C₁₋₄alkyl, cycloheteroalkyl, cycloheteroalkyl-C₁₋₄alkyl, aryl, aryl-C₁₋₄alkyl, aryloxy, arylthio, heteroaryl, and heteroaryl-C₁₋₄alkyl, wherein each alkyl is optionally substituted with one to four substituents independently selected from R^{a}, and each cycloalkyl and cycloheteroalkyl are optionally substituted with one to four substituents selected from R^{b} and oxo, and each aryl and heteroaryl are optionally substituted with one to four substituents independently selected from R^{b};

R^{3a} and R^{3b} are each independently selected from: hydrogen, aryl, and C₁₋₄alkyl, wherein each alkyl is optionally substituted with one to four substituents independently selected from R^{a};

R^{4a} and R^{4b} are each independently selected from: hydrogen, C₁₋₈alkyl, C₃₋₈cycloalkyl, C₃₋₈cycloalkyl-C₁₋₄alkyl, cycloheteroalkyl, cycloheteroalkyl-C₁₋₄alkyl, aryl, aryl-C₁₋₄alkyl, heteroaryl, and heteroaryl-C₁₋₄alkyl, or R^{4a} and R^{4b} together with the carbon to which they are attached form a carbocylic ring of 3 to 7 members containing 0-2 additional heteroatoms independently selected from oxygen, sulfur and N—Rg, wherein each alkyl is optionally substituted with one to four substituents independently selected from R^{a}, and each cycloalkyl and cycloheteroalkyl are optionally substituted with one to four substituents selected from R^{b} and oxo, and each aryl and heteroaryl are optionally substituted with one to four substituents independently selected from R^{b},

PROVIDED that R^{4a} and R^{4b} are not both hydrogen;

R⁵ is selected from: C₁₋₁₀alkyl, C₂₋₁₀alkenyl, C₂₋₁₀alkynyl, C₃₋₁₀cycloalkyl, C₃₋₁₀ cycloheteroalkyl, cycloheteroalkyl-C₁₋₄alkyl, aryl, aryl-C₁₋₄alkyl, heteroaryl, heteroaryl-C₁₋₄alkyl, —OR^{e}, and —NR^{c}R^{d}, wherein alkyl, alkenyl, cycloalkyl, and cycloheteroalkyl are optionally substituted with one to four substituents independently selected from R^{a} and each cycloalkyl and cycloheteroalkyl are optionally substituted with one to four substituents selected from R^{b} and oxo, and each aryl and heteroaryl are optionally substituted with one to four substituents independently selected from R^{b};

each R^{a} is independently selected from: —OR^{e}, —NR^{c}S(O)ₘR^{e}, halogen, —SR^{e}, —S(O)ₘNR^{c}R^{d}, —NR^{c}R^{d}, —C(O)R^{e}, —OC(O)R^{e}, —CO₂R^{e}, —CN, —C(O)NR^{c}R^{d}, —NR^{c}C(O)R^{e}, —NR^{c}C(O)OR^{e}, —NR^{c}C(O)NR^{c}R^{d}, —CF₃, and —OCF₃;
each R^{b} is independently selected from: R^{a}, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein alkyl and alkenyl moieties are unsubstituted or substituted with one, two, three or four R^{k} substituents, and cycloalkyl, cycloheteroalkyl, aryl and heteroaryl moieties are unsubstituted or substituted with one, two or three R^{k} substituents;

R^{c} and R^{d} are independently selected from: hydrogen, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₄alkyl, or R^{c} and R^{d} together with the atom(s) to which they are attached form a heterocyclic ring of 4 to 7 members containing 0-2 additional heteroatoms independently selected from oxygen, sulfur and N—Rg, wherein when R^{c} is not hydrogen, each R^{c} may be optionally or substituted with one to three substituents selected from R^{f}, and wherein when R^{d} is not hydrogen, each R^{d} may be optionally substituted with one to three substituents selected from R^{f};

each R^{e} is independently selected from: hydrogen, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein, when R^{e} is other than hydrogen, each R^{e} may be unsubstituted or substituted with one to three substituents selected from R^{f};

each R^{f} is independently selected from: halogen, C₁₋₁₀alkyl, C₂₋₁₀alkenyl, —CN, —CF₃, —OCF₃, —OR^{h}, —NHS(O)ₘR^{h}, —SR^{h}, —S(O)ₘNR^{h}R^{h}, —NR^{h}R^{h}, —C(O)R^{h}, —CO₂R^{h}, —C(O)NR^{h}R^{h}, —NR^{h}C(O)R^{h}, —NR^{h}C(O)OR^{h}, —NR^{h}C(O)NR^{h}R^{h}, cycloheteroalkyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl;

each Rg is independently selected from: C₁₋₁₀alkyl, and —C(O)R^{c};

each R^{h} is independently selected from: hydrogen, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein when R^{h} is not hydrogen, each R^{h} may be unsubstituted or substituted with one, two or three substituents selected from Rⁱ;

each Rⁱ is independently selected from: halogen, C₁₋₁₀alkyl, —O—C₁₋₄alkyl, —OH, —S—C₁₋₄alkyl, —CN, —CF₃, and —OCF₃;

each R^{k} is independently selected from: halogen, C₁₋₄alkyl, —O—C₁₋₄alkyl, —S—C₁₋₄alkyl, —CN, —CF₃, and —OCF₃; and

m is selected from 1 and 2;

PROVIDED that, when R² is unsubstituted alkyl and R⁵ is unsubstituted alkyl, then R¹ is not unsubstituted alkyl.

In one embodiment, R¹ is selected from: C₁₋₁₀alkyl, C₃₋₁₀cycloalkyl, C₃₋₁₀cycloalkyl-C₁₋₄alkyl, cycloheteroalkyl, cycloheteroalkyl-C₁₋₄alkyl, aryl, aryl-C₁₋₄alkyl, heteroaryl, heteroaryl-C₁₋₄alkyl, —OR^{e}, —NR^{c}R^{d}, —NR^{c}C(O)R^{e}, —CO₂R^{e}, and —C(O)NR^{c}R^{d}, wherein each alkyl is optionally substituted with one to four substituents independently selected from R^{a}, and each cycloalkyl and cycloheteroalkyl are optionally substituted with one to four substituents selected from R^{b} and oxo, and each aryl and heteroaryl are optionally substituted with one to four substituents independently selected from R^{b}.

In another embodiment, R¹ is selected from: C₁₋₁₀alkyl, C₃₋₁₀cycloalkyl, cycloheteroalkyl, aryl, heteroaryl, —OR^{e}, —NR^{c}R^{d}, —NR^{c}C(O)R^{e}, —CO₂R^{e}, and —C(O)NR^{c}R^{d}, wherein each alkyl is optionally substituted with one to three substituents independently selected from R^{a}, and each cycloalkyl and cycloheteroalkyl are unsubstituted or substituted with one to three substituents selected from R^{b} and oxo, and each aryl and heteroaryl are unsubstituted with substituted with one to four substituents independently selected from R^{b}.

In one class of this embodiment, R¹ is selected from: C₁₋₅alkyl, cycloalkyl, cycloheteroalkyl, aryl, heteroaryl, —OR^{e}, —NR^{c}R^{d}, and —CO₂R^{e},

wherein each alkyl is optionally substituted with one to three substituents independently selected from R^{a}, and each aryl and heteroaryl is optionally substituted with one to three substitutents independently selected from R^{b}.

In a subclass of this class, R¹ is selected from: C₁₋₅alkyl, cyclobutyl, cyclopentyl, cyclohexyl, pyrrolidinyl, phenyl, pyridyl, indolinyl, benzisoxazolyl, azaindolyl, 2,3-dihydroindolyl, 3,4-dihydroquinolinyl, pyridazinyl, pyrimidinyl, pyrrolidinyl, triazolyl, benzotriazolyl, thienyl, indolyl, indazolyl, —OR^{e}, —NR^{c}R^{d}, —C(O)OR^{e},

wherein each alkyl is optionally substituted with one or two R^{a} substituents and each aryl, cycloalkyl, heteroaryl, or cycloheteroaryl is independently with one to three R^{b} substituents.

In another subclass of this class, R¹ is selected from:
(1) ethyl,
(2) isopropyl,
(3) cyclopentyl,
(4) phenyl, unsubstituted or substituted with one or two substituents selected from: halo, methylthio-, methoxy-, C₁₋₃alkyl, cyano, trifluoromethyl, tetrazolyl, and 2H-tetrazolyl;
(5) pyridyl, unsubstituted or substituted with one or two substituents selected from: halo, methylthio-, methoxy-, C₁₋₃alkyl, cyano, trifluoromethyl, tetrazolyl, and 2H-tetrazolyl; pyrrolidinyl,
(6) indolinyl,
(7) benzisoxazolyl,
(8) azaindolyl
(9) 2,3-dihydro-1H-indolyl,
(10) 3,4-dihydroquinolinyl,
(11) pyridazinyl,
(12) pyrimidinyl,
(13) pyrrolidinyl,
(14) 1,2,3-triazolyl,
(15) 1,2,4-triazolyl,
(16) 1,2,3-benzotriazolyl,
(17) thienyl,
(18) 1H-indolyl, unsubstituted or substituted with methyl,
(19) 1H-indazolyl,
(20) methyloxy,
(21) ethyloxy,
(22) n-propyloxy,
(23) n-pentyloxy,
(24) cyclobutylmethyloxy,
(25) cyclopentylmethyloxy,
(26) (N-methyl-N-phenyl)amino, and
(27) phenylmethyloxycarbonyl.

In one class of this embodiment, R¹ is selected from: cycloalkyl, cycloheteroalkyl, aryl, heteroaryl, —OR^{e}, —NR^{c}R^{d}, and —CO₂R^{e}, wherein each alkyl is optionally substituted with one to three substituents independently selected from R^{a}, and each aryl and heteroaryl is optionally substituted with one to three substitutents independently selected from R^{b}.

In a subclass of this class, R¹ is selected from: cyclobutyl, cyclopentyl, cyclohexyl, pyrrolidinyl, phenyl, pyridyl, indolinyl, benzisoxazolyl, azaindolyl, 2,3-dihydroindolyl, 3,4-dihydroquinolinyl, pyridazinyl, pyrimidinyl, pyrrolidinyl, triazolyl, benzotriazolyl, thienyl, indolyl, indazolyl, —OR^{e}, —NR^{c}R^{d}, —C(O)OR^{e}, wherein each cycloalkyl and cycloheteroalkyl moiety is optionally substituted with one to three substituents selected from R^{b} and oxo, and each aryl and heteroaryl moiety is optionally substituted with one to four substituents independently selected from R^{b}.

In another subclass of this class, R¹ is selected from:
(1) cyclopentyl,
(2) phenyl, unsubstituted or substituted with one or two substituents selected from: halo, methylthio-, methoxy-, C₁₋₃alkyl, cyano, trifluoromethyl, tetrazolyl, and 2H-tetrazolyl;
(3) pyridyl, unsubstituted or substituted with one or two substituents selected from: halo, methylthio-, methoxy-, C1-3alkyl, cyano, trifluoromethyl, tetrazolyl, and 2H-tetrazolyl; pyrrolidinyl,
(4) indolinyl,
(5) benzisoxazolyl,
(6) azaindolyl
(7) 2,3-dihydro-1H-indolyl,
(8) 3,4-dihydroquinolinyl,
(9) pyridazinyl,
(10) pyrimidinyl,
(11) pyrrolidinyl,
(12) 1,2,3-triazolyl,
(13) 1,2,4-triazolyl,
(14) 1,2,3-benzotriazolyl,
(15) thienyl,
(16) 1H-indolyl, unsubstituted or substituted with methyl,
(17) 1H-indazolyl,
(18) methyloxy,
(19) ethyloxy,
(20) n-propyloxy,
(21) n-pentyloxy,
(22) cyclobutylmethyloxy,
(23) cyclopentylmethyloxy,
(24) (N-methyl-N-phenyl)amino, and
(25) phenylmethyloxycarbonyl,

In yet another subclass of this class, R¹ is 3-substituted phenyl, wherein the substituent is selected from R^{b}. In still another subclass of this class, R¹ is selected from: 3-cyanophenyl, and 3-(2H-tetrazol-5-yl)-phenyl.

In another subclass of this, R¹ is 3-cyanophenyl.

In one embodiment, R² is selected from: C₁₋₁₀alkyl, C₃₋₁₀cycloalkyl-C₁₋₄alkyl, cycloheteroalkyl, cycloheteroalkyl-C₁₋₄alkyl, aryl, aryl-C₁₋₄alkyl, aryloxy, arylthio, heteroaryl, and heteroaryl-C₁₋₄alkyl, wherein each alkyl is optionally substituted with one to four substituents independently selected from R^{a}, and each cycloalkyl, cycloheteroalkyl, aryl and heteroaryl is optionally substituted with one to four substituents independently selected from R^{b}.

In one class of this embodiment, R² is selected from: C₁₋₅alkyl, C₃₋₆cycloalkyl-C₁₋₄-alkyl, cycloheteroalkyl-C₁₋₄-alkyl, phenyl, phenyl-C₁₋₄alkyl, phenyloxy, phenylthio, pyridyl, and pyridyl-C₁₋₄alkyl, wherein each alkyl is optionally substituted with one or two substituents independently selected from R^{a}, and each cycloalkyl and cycloheteroalkyl is optionally unsubstituted or substituted with one to four substituents selected from R^{b} and oxo, and each aryl and heteroaryl is unsubstituted or substituted with one to four substituents independently selected from R^{b}.

In one subclass, R² is selected from: C₃₋₅alkyl, C₃₋₆cycloalkyl-methyl-, piperidinyl-methyl, phenyl, benzyl, phenyloxy, phenylthio, pyridyl, and pyridyl-methyl, wherein each alkyl is optionally substituted with one or two substituents independently selected from halogen, and each cycloalkyl, piperidinyl, phenyl and pyridyl is unsubstituted or substituted with one or two substituents independently selected from R^{b}.

In one subclass, R² is selected from: C₃₋₅alkyl, C₃₋₆cycloalkyl-methyl-, piperidinyl-methyl, phenyl, benzyl, phenyloxy, phenylthio, pyridyl, and pyridyl-methyl, wherein each piperidinyl, phenyl and pyridyl is unsubstituted or substituted with one or two substituents independently selected from: halo-, methoxy, methoxycarbonyl, cyano, methyl, and t-butyloxycarbonyl.

In still another subclass, R² is selected from:
(1) 2-methylpropyl,
(2) cyclopropylmethyl,
(3) cyclobutylmethyl,
(4) cyclopentylmethyl,
(5) cyclohexylmethyl,
(6) 4-t-butyloxycarbonylpiperidin-4-yl-methyl,
(7) 4-substituted phenyl, wherein the substituent is selected from fluoro, chloro, methyl, methoxy, methoxycarbonyl, and cyano,
(8) benzyl unsubstituted or substituted with one or two substituents independently selected from fluoro, chloro, methyl, methoxy, methoxycarbonyl, and cyano,
(9) 4-substituted phenyloxy-, wherein the phenyl substituent is selected from fluoro, chloro, methyl, methoxy, methoxycarbonyl, and cyano,
(10) 4-substituted phenylthio-, wherein the phenyl substituent is selected from fluoro, chloro, methyl, methoxy, methoxycarbonyl, and cyano, and
(11) 5-substituted-2-pyridyl-methyl-, wherein the pyridyl substituent is selected from fluoro, chloro, methyl, methoxy and cyano.

In yet another subclass, R² is benzyl, unsubstituted or substituted with one or two substituents independently selected from fluoro, chloro, methyl, methoxy, methoxycarbonyl, and cyano.

In another subclass, R² is 4-substituted benzyl, wherein the substituent is selected from fluoro, chloro, methyl, methoxy, methoxycarbonyl, and cyano.

In yet another subclass, R² is 4-chlorobenzyl.

In one embodiment, R^{3a} is independently selected from: hydrogen, aryl, and C₁₋₄alkyl, wherein each alkyl is optionally substituted with one to four substituents independently selected from R^{a}.

In one class of this embodiment, R^{3a} is selected from: hydrogen, phenyl, and C₁₋₄alkyl, wherein each alkyl is unsubstituted or substituted with R^{a}.

In another class of this embodiment, R^{3a} is selected from: hydrogen, phenyl, methyl, and ethyl.

In still another class, R^{3a} is selected from: hydrogen, and methyl.

In a subclass, R^{3a} is methyl.

In one embodiment of the present invention, R^{3b} is selected from: hydrogen, aryl, and C₁₋₄alkyl, wherein each alkyl is optionally substituted with one to four substituents independently selected from R^{a}.

In one class of this embodiment, R^{3b} is selected from: hydrogen, phenyl, and C₁₋₄alkyl, wherein each alkyl is unsubstituted or substituted with R^{a}.

In another class of this embodiment, R^{3b} is selected from: hydrogen, phenyl, methyl, and ethyl.

In still another class, R^{3b} is selected from: hydrogen, and methyl.

In a subclass, R^{3b} is hydrogen.

In one embodiment, R^{4a} and R^{4b} are each independently selected from: hydrogen, C₁₋₈alkyl C₃₋₈cycloalkyl, C₃₋₈cycloalkyl-C₁₋₄alkyl, cycloheteroalkyl, cycloheteroalkyl-C₁₋₄alkyl, aryl, aryl-C₁₋₄alkyl, heteroaryl, and heteroaryl-C₁₋₄alkyl, or

R^{4a} and R^{4b} together with the carbon to which they are attached form a carbocylic ring of 3 to 7 members containing 0-2 additional heteroatoms independently selected from oxygen, sulfur and N—Rg, wherein each alkyl is optionally substituted with one to four substituents independently selected from R^{a}, and each and each cycloalkyl and cycloheteroalkyl are optionally substituted with one to four substituents selected from R^{b} and oxo, and each aryl and heteroaryl are optionally substituted with one to four substituents independently selected from R^{b}, PROVIDED that R^{4a}, and R^{4b} are not both hydrogen.

In one class, R^{4a} and R^{4b} are each independently selected from: hydrogen, C₁₋₆alkyl, C₃₋₇cycloalkyl, C₃₋₇cycloalkyl-methyl-, cycloheteroalkyl, cycloheteroalkyl-methyl-, aryl, aryl-methyl-, heteroaryl, and heteroaryl-C₁₋₄alkyl, or R^{4a} and R^{4b} together with the carbon to which they are attached form a carbocylic ring of 3 to 7 members, wherein each alkyl is unsubstituted or substituted with one or two substituents independently selected from R^{a}, and each cycloalkyl and cycloheteroalkyl are optionally substituted with one or two substituents selected from R^{b} and oxo, and each aryl and heteroaryl are optionally substituted with one or two substituents independently selected from R^{b}, PROVIDED that R^{4a} and R^{4b} are not both hydrogen.

In another class, R^{4a} and R^{4b} are each independently selected from: hydrogen, C₁₋₆alkyl, phenyl, phenyl-methyl-, or R^{4a} and R^{4b} together with the carbon to which they are attached form a cyclopropyl ring, wherein each alkyl is unsubstituted or substituted with one or two substituents independently selected from R^{a}, and each phenyl moiety is unsubstituted or substituted with one or two substituents independently selected from R^{b}, PROVIDED that R^{4a} and 124.13 are not both hydrogen.

In a subclass, R^{4a} and R^{4b} are each independently selected from: hydrogen, methyl, ethyl, isopropyl, t-butyl, phenyl, phenyl-methyl-, or R^{4a} and R^{4b} together with the carbon to which they are attached form a cyclopropyl ring, PROVIDED that R^{4a} and R^{4b} are not both hydrogen.

In one embodiment, R⁵ is selected from: C₁₋₁₀alkyl, C₂₋₁₀alkenyl, C₂₋₁₀alkynyl, C₃₋₁₀cycloalkyl, C₃₋₁₀cycloalkyl-C₁₋₄alkyl, cycloheteroalkyl, cycloheteroalkyl-C₁₋₄alkyl, aryl, aryl-C₁₋₄alkyl, heteroaryl, heteroaryl-C₁₋₄alkyl, —OR^{e}, and —NR^{c}R^{d}; wherein alkyl, alkenyl, and alkenyl are unsubstituted or substituted with one to four substituents independently selected from R^{a}, and each cycloalkyl and cycloheteroalkyl are optionally substituted with one to four substituents selected from R^{b} and oxo, and each aryl and heteroaryl are optionally substituted with one to four substituents independently selected from R^{b}.

In one class of this embodiment, R⁵ is selected from: C₁₋₆alkyl, C₂₋₆alkenyl, C₂₋₆alkynyl, C₃₋₈cycloalkyl, C₃₋₈cycloalkyl-C₁₋₄alkyl, cycloheteroalkyl, cycloheteroalkyl-C₁₋₄-alkyl, aryl, aryl-C₁₋₄alkyl, heteroaryl, heteroaryl-C₁₋₄alkyl; —NR^{c}R^{d}, wherein alkyl, alkenyl and alkynyl, are unsubstituted or substituted with one to three substituents independently selected from R^{a}, and each cycloalkyl and cycloheteroalkyl are optionally substituted with one to three substituents selected from R^{b} and oxo, and each aryl and heteroaryl are optionally substituted with one to three substituents independently selected from R^{b}.

In another class, R⁵ is selected from: C₁₋₆alkyl, C₁₋₆cycloalkyl, C₁₋₆cycloalkyl-methyl-, cycloheteroalkyl, cycloheteroalkyl-methyl-, aryl, aryl-methyl-, heteroaryl, heteroaryl-methyl-, —NHR^{d}, wherein alkyl is unsubstituted or substituted with one or two substituents independently selected from R^{a}, and each cycloalkyl and cycloheteroalkyl are optionally substituted with one to three substituents selected from R^{b} and oxo, and each aryl and heteroaryl are optionally substituted with one to three substituents independently selected from R^{b}.

In another class, R⁵ is selected from: C₁₋₆alkyl, C₃₋₆cycloalkyl, C₃₋₆cycloalkyl-methyl-, cycloheteroalkyl, cycloheteroalkyl-methyl-, phenyl, phenyl-methyl-, heteroaryl, heteroaryl-methyl-, —NHR^{d}, wherein alkyl is optionally substituted with one to three substituents independently selected from R^{a}, and each cycloalkyl and cycloheteroalkyl are optionally substituted with one to three substituents selected from R^{b} and oxo, and each aryl and heteroaryl are optionally substituted with one to three substituents independently selected from R^{b}.

In still another class, R⁵ is selected from: C₁₋₆cycloalkyl, C₃₋₆cycloalkyl-methyl-, cycloheteroalkyl, cycloheteroalkyl-methyl-, phenyl, phenyl-methyl-, heteroaryl, heteroaryl-methyl-, —NHR^{d}, wherein alkyl is optionally substituted with one to three substituents independently selected from R^{a}, and each cycloalkyl and cycloheteroalkyl are optionally substituted with one to three substituents selected from R^{b} and oxo, and each aryl and heteroaryl are optionally substituted with one to three substituents independently selected from R^{b}.

In another class, R⁵ is selected from: methyl, ethyl, isopropyl, 2,2-dimethylpropyl, t-butyl, cyclobutyl, cyclopentyl, cyclohexyl, tetrahydrofuranyl, phenyl, phenyl-methyl-, pyridyl, pyridylmethyl, thiadiazolyl, pyrazolyl, pyrazinyl, isoxazolyl, thiazolyl, pyrimidinyl, oxazolyl, oxadiazolyl, triazolyl, imidazolyl, pyrazolo[1,5-a]pyrimidinyl, imidazo[2,1-b]thiazolyl, imidazo[1,2-a]pyridyl, anilino, wherein alkyl moieties are optionally substituted with one to three substituents independently selected from R^{a}, and each cycloalkyl and cycloheteroalkyl are optionally substituted with one to three substituents selected from R^{b} and oxo, and each aryl and heteroaryl are optionally substituted with one to three substituents independently selected from R^{b}.

In still another class, R⁵ is selected from: C₃₋₆cycloalkyl, C₃₋₆cycloalkyl-methyl-, cycloheteroalkyl, cycloheteroalkyl-methyl-, phenyl, phenyl-methyl-, heteroaryl, heteroaryl-methyl-, —NHR^{d}; wherein alkyl is optionally substituted with one to three substituents independently selected from R^{a}, and each cycloalkyl and cycloheteroalkyl are optionally substituted with one to three substituents selected from R^{b} and oxo, and each aryl and heteroaryl are optionally substituted with one to three substituents independently selected from R^{b}.

In yet another class, R⁵ is selected from: cyclobutyl, cyclopentyl, cyclohexyl, tetrahydrofuranyl, phenyl, phenyl-methyl-, pyridyl, pyridylmethyl, thiadiazolyl, pyrazolyl, pyrazinyl, isoxazolyl, thiazolyl, pyrimidinyl, oxazolyl, oxadiazolyl, triazolyl, imidazolyl, pyrazolo[1,5-a]pyrimidinyl, imidazo[2,1-b]thiazolyl, imidazo[1,2-a]pyridyl, anilino, wherein alkyl moieties are optionally substituted with one to three substituents independently selected from R^{a}, and each cycloalkyl and cycloheteroalkyl are optionally substituted with one to three substituents selected from R^{b} and oxo, and each aryl and heteroaryl are optionally substituted with one to three substituents independently selected from R^{b}.

In one embodiment, each R^{a} is independently selected from: —OR^{e}, —NR^{c}S(O)ₘR^{e}, halogen, —SR^{e}, —S(O)ₘNR^{c}R^{d}, —C(O)R^{e}, —OC(O)R^{e}, —CO₂R^{e}, —CN, —C(O)NR^{c}R^{d}, NR^{c}C(O)R^{e}, —NR^{c}C(O)OR^{e}, —NR^{c}C(O)NR^{c}R^{d}, —CF₃, and —OCF₃.

In one class, each R^{a} is independently selected from: —OR^{e}, —NHS(O)₂R^{e}, halogen, —SR^{e}, —S(O)₂NR^{c}R^{d}, —NR^{c}R^{d}, —C(O)R^{e}, —OC(O)R^{e}, —CO₂R^{e}, —CN, —C(O)NR^{c}R^{d}, —NHC(O)R^{e}, —NHC(O)OR^{e}, —NHC(O)NR^{c}R^{d}, —CF₃, and —OCF₃.

In another class, each R^{a} is independently selected from: hydroxy, methoxy, methylcarbonyloxy, fluoro, chloro, methylthio, amino, N,N-dimethylamino, N-methylamino, methylcarbonyl, methoxycarbonyl, —CN, N-methylcarbonyl-amino-, N-(t-butyloxycarbonyl)amino-, —CF₃, and —OCF₃.

In yet another class, each R^{a} is independently selected from: hydroxy, methoxy, methylcarbonyloxy, fluoro, chloro, N-(t-butyloxycarbonyl)amino-, and —OCF₃.

In still another class, each R^{a} is independently selected from: hydroxy, methylcarbonyloxy, and N-(t-butyloxycarbonyl)amino-.

In one embodiment, each R^{b} is independently selected from: —OR^{e}, —NR^{c}S(O)ₘR^{e}, halogen, —SR^{e}, —S(O)ₘNR^{c}R^{d}, —NR^{c}R^{d}, —C(O)R^{e}, —OC(O)R^{e}, —CO₂R^{e}, —CN, —C(O)NR^{c}R^{d}, —NR^{c}C(O)R^{e}, —NR^{c}C(O)OR^{e}, —NR^{c}C(O)NR^{c}R^{d}, —CF₃, —OCF₃, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl; wherein alkyl and alkenyl moieties are unsubstituted or substituted with one, two, three or four R^{k} substituents, and cycloalkyl, cycloheteroalkyl, aryl and heteroaryl moieties are unsubstituted or substituted with one, two or three R^{k} substituents.

In one class of this embodiment, each R^{b} is independently selected from: —OR^{e}, —NHS(O)₂R^{e}, halogen, —SCH₃, —S(O)₂NR^{c}R^{d}, —NR^{c}R^{d}, —C(O)R^{e}, —OC(O)R^{e}, —CO₂R^{e}, —CN, —C(O)NR^{c}R^{d}, NHC(O)R^{e}, —NHC(O)OR^{e}, —NHC(O)NR^{c}R^{d}, —CF₃, —OCF₃, C₁₋₆ alkyl, C₂₋₆alkenyl, cycloalkyl, cycloalkyl-methyl, cycloheteroalkyl, cycloheteroalkyl-methyl, phenyl, heteroaryl, phenylmethyl, and heteroaryl-methyl; wherein alkyl and alkenyl moieties are unsubstituted or substituted with one, two, three or four R^{k} substituents, and cycloalkyl, cycloheteroalkyl, aryl and heteroaryl moieties are unsubstituted or substituted with one, two or three R^{k} substituents.

In another class of this embodiment, each R^{b} is independently selected from: —OR^{e}, halogen, —SCH₃, —NR^{c}R^{d}, —C(O)CH₃, —OC(O)R^{e}, —CO₂R^{e}, —CN, —C(O)NR^{c}R^{d}, —NHC(O)R^{e}, —NHC(O)OR^{e}, —CF₃, —OCF₃, C₁₋₆alkyl, cycloalkyl, cycloheteroalkyl, phenyl, and heteroaryl; wherein alkyl is are unsubstituted or substituted with one, two, three or four R^{k} substituents, and cycloalkyl, cycloheteroalkyl, aryl and heteroaryl moieties are unsubstituted or substituted with one, two or three R^{k} substituents.

In yet another class, each R^{b} is independently selected from: —OH, —OCH₃, —OCH₂CF₃, —Cl, —F, —Br, —I, —SCH₃, —NH₂, —OC(O)CH₃, t-butyloxycarbonyl-, —CN, —NHC(O)CF₃, —CF₃, —OCF₃, methyl, ethyl, isopropyl, t-butyl, —CF₂—CF₂H, cyclopropyl, 2-H-tetrazolyl, cycloheteroalkyl, phenyl, Unsubstituted or substituted with a methyl or halogen substituent, and 1,2,3-thiazolyl unsubstituted or substituted with a methyl or halogen substituent.

In one embodiment, each R^{c} is independently selected from: hydrogen, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein when R^{c} is not hydrogen, each R^{c} may be optionally substituted with one to three substituents selected from R^{f}.

In one class of this embodiment, each R^{c} is independently selected from: hydrogen, C₁₋₆alkyl, C₂₋₆alkenyl, cycloalkyl, cycloalkyl-methyl, cycloheteroalkyl, cycloheteroalkyl-methyl, aryl, heteroaryl, aryl-methyl, and heteroaryl-methyl, wherein when R^{c} is not hydrogen, each R^{c} may be optionally substituted with one to three substituents selected from R^{f}.

In one another class, each R^{c} is independently selected from: hydrogen, C₁₋₆alkyl, cycloalkyl, cycloheteroalkyl, phenyl, and heteroaryl, wherein when R^{c} is not hydrogen, each R^{c} may be optionally substituted with one to three substituents selected from R^{f}.

In one subclass, each R^{c} is independently selected from: hydrogen, and methyl, wherein when R^{c} is not hydrogen, R^{c} may be optionally substituted with one to three substituents selected from R^{f}.

In one embodiment, each R^{d} is independently selected from: hydrogen, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein, when R^{d} is not hydrogen, each R^{d} may be optionally substituted with one to three substituents selected from R^{f}.

In a class of this embodiment, each R^{d} is independently selected from: hydrogen, C₁₋₆alkyl, C₂₋₆ alkenyl, cycloalkyl, cycloalkyl-methyl, cycloheteroalkyl, cycloheteroalkyl-methyl, aryl, heteroaryl, aryl-methyl, and heteroaryl-methyl; wherein, when R^{d} is not hydrogen, each R^{d} may be optionally substituted with one to three substituents selected from R^{f}.

In another class of this embodiment, each R^{d} is independently selected from: hydrogen, C₁₋₆alkyl, cycloalkyl, cycloheteroalkyl, aryl, and heteroaryl, wherein, when R^{d} is not hydrogen, each R^{d} may be optionally substituted with one to three substituents selected from R^{f}.

In still another class of this embodiment, each R^{d} is independently selected from: hydrogen, methyl, ethyl, isopropyl, t-butyl, cyclopropyl, cycloheteroalkyl, phenyl, and heteroaryl; wherein, when R^{d} is not hydrogen, each R^{d} may be optionally substituted with one to three substituents selected from R^{f}.

In a subclass of this class, each R^{d} is independently selected from: hydrogen, and methyl.

In one embodiment, R^{c} and R^{d} together with the atom(s) to which they are attached form a heterocyclic ring of 4 to 7 members containing 0-2 additional heteroatoms independently selected from oxygen, sulfur and N—Rg, wherein the heterocyclic ring formed by R^{c} and R^{d} may be unsubstituted or substituted with one to three substituents selected from R^{f}.

In one class, R^{c} and R^{d} together with the atom(s) to which they are attached form a heterocyclic ring of 4 to 7 members, wherein the heterocyclic ring formed by R^{c} and R^{d} may be unsubstituted or substituted with one to three substituents selected from R^{f}.

In one embodiment, each R^{e} is independently selected from: hydrogen, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein, when R^{e} is other than hydrogen, each R^{e} may be unsubstituted or substituted with one to three substituents selected from R^{f}.

In one class of this embodiment, each R^{e} is independently selected from: hydrogen, C₁₋₆alkyl, C₂₋₆alkenyl, cycloalkyl, cycloalkyl-methyl, cycloheteroalkyl, cycloheteroalkyl-methyl, aryl, heteroaryl, aryl-methyl, and heteroaryl-methyl, wherein, when R^{e} is other than hydrogen, each R^{e} may be unsubstituted or substituted with one to three substituents selected from R^{f}.

In another class, each R^{e} is independently selected from: hydrogen, C₁₋₆alkyl, cycloalkyl, cycloheteroalkyl, aryl, and heteroaryl, wherein, when R^{e} is other than hydrogen, each R^{e} may be unsubstituted or substituted with one to three substituents selected from R^{f}.

In a subclass, each R^{e} is independently selected from: hydrogen, methyl, ethyl, trifluoromethyl, —CH₂CF₃, and t-butyl.

In one embodiment of the present invention, each R^{f} is independently selected from: halogen, C₁₋₁₀alkyl, C₂₋₁₀ alkenyl, —CN, —CF₃, —OCF₃, —OR^{h}, —NHS(O)ₘR^{h}, —SR^{h}, —S(O)ₘNR^{h}R^{h}, —NR^{h}R^{h}, —C(O)R^{h}, —CO₂R^{h}, —C(O)NR^{h}R^{h}, —NR^{h}C(O)R^{h}, —NR^{h}C(O)OR^{h}, —NR^{h}C(O)NR^{h}R^{h}, cycloheteroalkyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl.

In one class, each R^{f} is independently selected from: halogen, C₁₋₆alkyl, C₂₋₆alkenyl, —S—CH₃, —CN, —CF₃, —OCF₃, —OR^{h}, —NHS(O)₂R^{h}, —S(O)₂NR^{h}R^{h}, —NR^{h}R^{h}, —C(O)R^{h}, —CO₂R^{h}, —C(O)NR^{h}R^{h}, —NHC(O)R^{h}, —NHC(O)OR^{h}, —NHC(O)NR^{h}R^{h}, cycloheteroalkyl, cycloalkyl, cycloalkyl-methyl-, cycloheteroalkyl, cycloheteroalkyl-methyl-, phenyl, heteroaryl, phenylmethyl, and heteroaryl-methyl-.

In another class, each R^{f} is independently selected from: halogen, C₁₋₆alkyl, —S—CH₃, —CN, —CF₃, —OCF₃, —OCH₃, —NHS(O)₂CH₃, —S(O)₂NH₂, —S(O)₂NHCH₃, —S(O)₂N(CH₃)₂, —NH₂, —NHCH₃, —N(CH₃)₂, —C(O)CH₃, —CO₂H, —CO₂CH₃, —CO₂C(CH₃)₃, —C(O)NH₂, —C(O)NHCH₃, —C(O)N(CH₃)₂, —NHC(O)CF₃, —NHC(O)CH₃, —NHC(O)H, —NHC(O)OH, —NHC(O)OCH₃, —NHC(O)NH₂, —NHC(O)—NHCH₃, —NHC(O)N(CH₃)₂, cycloheteroalkyl, cycloalkyl, cycloheteroalkyl, phenyl, and heteroaryl.

In yet another class, each R^{f} is independently selected from: —F, —Cl, —I, methyl, ethyl, isopropyl, t-butyl, —S—CH₃, —CN, —CF₃, —OCF₃, —OCH₃, —NH₂, —NHCH₃, —N(CH₃)₂, —C(O)CH₃, —CO₂CH₃, —CO₂C(CH₃)₃, 2H-tetrazolyl, cyclopropyl, phenyl, thiazolyl, and pyridyl.

In one embodiment, each Rg is independently selected from: C₁₋₁₀alkyl, and —C(O)R^{c}.

In one class, each Rg is independently selected from: C₁₋₄alkyl, and —C(O)C₁₋₄alkyl.

In another class, each Rg is methyl or methylcarbonyl.

In one subclass, each Rg is methyl.

In one embodiment of the present invention, each R^{h} is independently selected from: hydrogen, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein when R^{h} is not hydrogen, each R^{h} may be unsubstituted or substituted with one, two or three substituents selected from Rⁱ.

In a class of this embodiment, each R^{h} is independently selected from: hydrogen, C₁₋₆alkyl, C₂₋₆ alkenyl, cycloalkyl, cycloalkyl-methyl, cycloheteroalkyl, cycloheteroalkyl-methyl, aryl, heteroaryl, aryl-methyl, and heteroaryl-methyl, wherein, when R^{h} is not hydrogen, each R^{h} may be optionally substituted with one to three substituents selected from Rⁱ.

In another class of this embodiment, each R^{h} is independently selected from: hydrogen, C₁₋₆alkyl, cycloalkyl, cycloheteroalkyl, aryl, and heteroaryl, wherein, when R^{h} is not hydrogen, each R^{h} may be optionally substituted with one to three substituents selected from Rⁱ.

In still another class of this embodiment, each R^{h} is independently selected from: hydrogen, methyl, ethyl, isopropyl, t-butyl, cyclopropyl, cycloheteroalkyl, phenyl, and heteroaryl, wherein, when R^{h} is not hydrogen, each R^{h} may be optionally substituted with one to three substituents selected from Rⁱ.

In a subclass of this class, each R^{h} is independently selected from: hydrogen, and methyl.

In one embodiment, each Rⁱ is independently selected from: halogen, C₁₋₁₀ alkyl, —O—C₁₋₄alkyl, —OH, —S—C₁₋₄alkyl, —CN, —CF₃, and —OCF₃.

In one class of this embodiment, each Rⁱ is independently selected from: halogen, C₁₋₆alkyl, —O—CH₃, —S—CH₃, —CN, —CF₃, and —OCF₃.

In another class of this embodiment, each Rⁱ is independently selected from: halogen, C₁₋₄alkyl, —O—CH₃, —S—CH₃, —CN, —CF₃, and —OCF₃.

In another class of this embodiment, each Rⁱ is independently selected from: —F, —Cl, —CH₃, —O—CH₃, —S—CH₃, —CN, —CF₃, and —OCF₃.

In another embodiment, each R^{k} is independently selected from: halogen, C₁₋₄alkyl, —O—C₁₋₄alkyl, —S—C₁₋₄alkyl, —CN, —CF₃, and —OCF₃.

In one class, each R^{k} is independently selected from: halogen, C₁₋₄alkyl, —O—CH₃, —S—CH₃, —CN, —CF₃, and —OCF₃.

In another class, each R^{k} is independently selected from: —F, —Cl, and —CH₃.

In one embodiment, each m is selected from 1 and 2. In one class, m is 1. In another class, m is 2.

One embodiment of the present invention comprises compounds of structural formula IA:

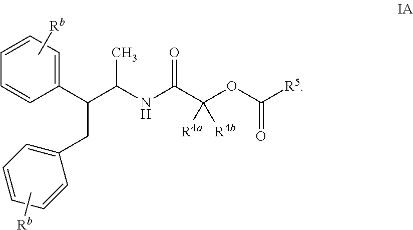

Another embodiment of the present invention comprises compounds of structural formula IB:

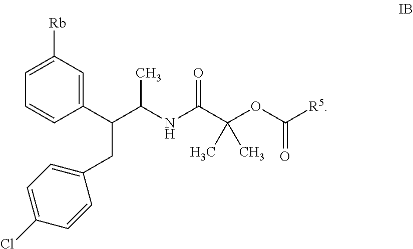

Still another embodiment of the present invention comprises compounds of structural formula IC:

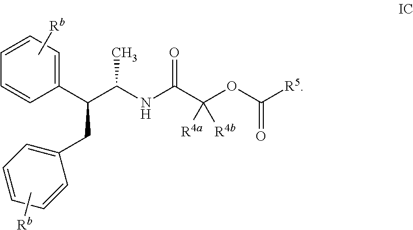

Another embodiment of the present invention comprises compounds of structural formula ID:

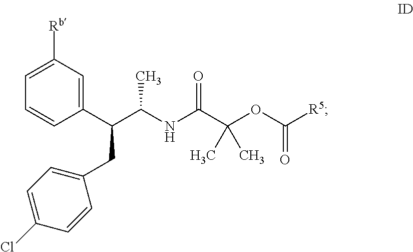

wherein Rb′ is selected from —CN, and

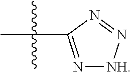

“Alkyl”, as well as other groups having the prefix “alk”, such as alkoxy, alkanoyl, means carbon chains which may be linear or branched or combinations thereof. Examples of alkyl groups include methyl, ethyl, n-propyl, isopropyl, butyl, isobutyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic or bridged saturated carbocyclic rings, each having from 3 to 10 carbon atoms. Examples of cycloalkyl include cyclopropyl, cyclobutyl, cyclopentyl, cyclohexyl, cycloheptyl, cyclooctyl, tetrahydronaphthyl, decahydronaphthyl, and the like. In one embodiment of the present invention, cycloalkyl is selected from cyclopropyl, cyclobutyl, cyclopentyl, and cyclohexyl.

“Aryl” means mono- or bicyclic aromatic rings containing only carbon atoms. Examples of aryl include phenyl, naphthyl, and the like. In one embodiment, aryl is phenyl.

“Heteroaryl” means a mono- or bicyclic aromatic ring containing at least one heteroatom selected from N, O and S, with each ring containing 5 to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzothiazolyl, furo(2,3-b)pyridyl, quinolyl, indolyl, isoquinolyl, oxazolidinyl, and the like. The heteroaryl ring may be substituted on one or more carbon atoms. In one embodiment of the present invention, heteroaryl is selected from pyridinyl, pyrazolyl, imidazolyl, pyrazinyl, pyridazinyl, pyrimidinyl, triazolyl, thienyl, 7-azaindolyl, benzisoxazolyl, indolinyl, indolyl, indazolyl, isoxazolyl, oxazolyl, tetrazolyl, imidazothiazolyl, imidazolpyridyl, pyrazolylpyridyl, and benzotriazolyl.

“Cycloheteroalkyl” means mono- or bicyclic or bridged saturated rings containing at least one heteroatom selected from N, S and O, each of said ring having from 3 to 10 atoms in which the point of attachment may be carbon or nitrogen. Examples of “cycloheteroalkyl” include pyrrolidinyl, piperidinyl, piperazinyl, imidazolidinyl, pyranyl, tetrahydrofuranyl, morpholinyl, dioxanyl, oxanyl, azetidinyl, perhydroazepinyl, tetrahydrofuranyl, 1-thia-4-aza-cyclohexane (thiomorpholinyl), hexahydrothieno-pyridinyl, thienopyridinyl, azacycloheptyl, and the like. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The cycloheteroalkyl ring may be substituted on the ring carbons and/or the ring nitrogens. In one embodiment of the present invention, cycloheteroalkyl is selected from furanyl, thiadiazolyl, piperidinyl, pyrrolidinyl, dihydroquinolinyl, and dihydroindolyl.

“Halogen” includes fluorine, chlorine, bromine and iodine.

When any variable (e.g., R¹, R^{d}, etc.) occurs more than one time in any constituent or in formula I, its definition on each occurrence is independent of its definition at every other occurrence. Also, combinations of substituents and/or variables are permissible only if such combinations result in stable compounds. A squiggly line across a bond in a substituent variable represents the point of attachment.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to:

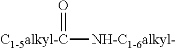

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Compounds of Formula I may contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I.

Compounds of the Formula I may be separated into diastereoisomeric pairs of enantiomers by, for example, fractional crystallization from a suitable solvent, for example MeOH or ethyl acetate or a mixture thereof. The pair of enantiomers thus obtained may be separated into individual stereoisomers by conventional means, for example by the use of an optically active amine as a resolving agent or on a chiral HPLC column.

Alternatively, any enantiomer of a compound of the general Formula I may be obtained by stereospecific synthesis using optically pure starting materials or reagents of known configuration.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (emboriate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to the compounds of Formula I are meant to also include the pharmaceutically acceptable salts.

Compounds of the present invention are modulators of the CB1 receptor. In particular, the compounds of structural formula I are antagonists or inverse agonists of the CB1 receptor.

An “agonist” is a compound (hormone, neurotransmitter or synthetic compound) which binds to a receptor and mimics the effects of the endogenous regulatory compound, such as contraction, relaxation, secretion, change in enzyme activity, etc. An “antagonist” is a compound, devoid of intrinsic regulatory activity, which produces effects by interfering with the binding of the endogenous agonist or inhibiting the action of an agonist. An “inverse agonist” is a compound which acts on a receptor but produces the opposite effect produced by the agonist of the particular receptor.

Compounds of this invention are modulators of the CB1 receptor and as such are useful as centrally acting drugs in the treatment of psychosis, memory deficits, cognitive disorders, Alzheimer's disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. In particular, the compounds of this invention are antagonists/inverse agonists of the CB1 receptor. The compounds are also useful for the treatment of substance abuse disorders, particularly to opiates, alcohol, marijuana, and nicotine. In particular, the compounds of the invention are useful for smoking cessation. The compounds are also useful for the treatment of obesity or eating disorders associated with excessive food intake and complications associated therewith, including left ventricular hypertrophy, as well as treating or preventing obesity in other mammalian species, including canines and felines. The compounds are also useful for the treatment of constipation and chronic intestinal pseudo-obstruction. The compounds are also useful for the treatment of cirrhosis of the liver, non-alcoholic fatty liver disease (NAFLD), non-alcoholic steatohepatitis (NASH) promotion of wakefulness and treatment of asthma.

The terms “administration of” and or “administering a” compound should be understood to mean providing a compound of the invention or a prodrug of a compound of the invention to the individual in need of treatment.

The administration of the compound of structural formula I in order to practice the present methods of therapy is carried out by administering an effective amount of the compound of structural formula I to the mammalian patient in need of such treatment or prophylaxis. The need for a prophylactic administration according to the methods of the present invention is determined via the use of well known risk factors. The effective amount of an individual compound is determined, in the final analysis, by the physician or veterinarian in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The usefulness of the present compounds in these diseases or disorders may be demonstrated in animal disease models that have been reported in the literature. The following are examples of such animal disease models: a) suppression of food intake and resultant weight loss in rats (Life Sciences 1998, 63, 113-117); b) reduction of sweet food intake in marmosets (Behavioural Pharm. 1998, 9, 179-181); c) reduction of sucrose and ethanol intake in mice (Psychopharm. 1997, 132, 104-106); d) increased motor activity and place conditioning in rats (Psychopharm. 1998, 135, 324-332; Psychopharmacol 2000, 151: 25-30); e) spontaneous locomotor activity in mice (J. Pharm. Exp. Ther. 1996, 277, 586-594); f) reduction in opiate self-administration in mice (Sci. 1999, 283, 401-404); g) bronchial hyperresponsiveness in sheep and guinea pigs as models for the various phases of asthma (for example, see W. M. Abraham et al., “α₄-Integrins mediate antigen-induced late bronchial responses and prolonged airway hyperresponsiveness in sheep.” J. Clin. Invest. 93, 776 (1993) and A. A. Y. Milne and P. P. Piper, “Role of VLA-4 integrin in leucocyte recruitment and bronchial hyperresponsiveness in the guinea-pig.” Eur. J. Pharmacol., 282, 243 (1995)); h) mediation of the vasodilated state in advanced liver cirrhosis induced by carbon tetrachloride (Nature Medicine, 2001, 7 (7), 827-832); i) amitriptyline-induced constipation in cynomolgus monkeys is beneficial for the evaluation of laxatives (Biol. Pharm. Bulletin (Japan), 2000, 23(5), 657-9); j) neuropathology of paediatric chronic intestinal pseudo-obstruction and animal models related to the neuropathology of paediatric chronic intestinal pseudo-obstruction (Journal of Pathology (England), 2001, 194 (3), 277-88).

The magnitude of prophylactic or therapeutic dose of a compound of Formula I will, of course, vary with the nature of the severity of the condition to be treated and with the particular compound of Formula I and its route of administration. It will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.001 mg to about 100 mg in one embodiment from about 0.01 mg to about 50 mg, and in another embodiment from 0.1 mg to 10 mg of a compound of Formula I per kg of body weight per day.

In the case where an oral composition is employed, a suitable dosage range is, e.g. from about 0.01 mg to about 1000 mg of a compound of Formula I per day. In one embodiment, the range is from about 0.1 mg to about 10 mg per day. For oral administration, the compositions are preferably provided in the form of tablets containing from 0.01 to 1,000 mg, preferably 0.01, 0.05, 0.1, 0.5, 1, 2, 2.5, 3, 4, 5, 6, 7, 8, 9, 10, 12, 12.5, 15, 20, 25, 30, 40, 50, 100, 250, 500, 750 or 1000 milligrams of the active ingredient for the symptomatic adjustment of the dosage to the patient to be treated.

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, particularly a human or companion animal such as a dog or cat, with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers, or as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, solutions, ointments, gels, lotions, dusting powders, and the like. The topical pharmaceutical compositions containing the compounds of the present invention ordinarily include about 0.005% to 5% by weight of the active compound in admixture with a pharmaceutically acceptable vehicle. Transdermal skin patches useful for administering the compounds of the present invention include those well known to those of ordinary skill in that art.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredient, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet cachet or capsule contains from about 0.01 to 1,000 mg, particularly 0.01, 0.05, 0.1, 0.5, 1.0, 2, 2.5, 3, 4, 5, 6, 7, 8, 9, 10, 12, 15, 25, 30, 40, 50, 75, 100, 125, 150, 175, 180, 200, 225, 250, 500, 750 and 1,000 milligrams of the active ingredient for the symptomatic adjustment of the dosage to the patient to be treated.

Additional suitable means of administration of the compounds of the present invention include injection, intravenous bolus or infusion, intraperitoneal, subcutaneous, intramuscular and topical, with or without occlusion.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compounds of Formula I:

| Injectable | | | mg/ |
|---|---|---|---|
| Suspension (I.M.) | mg/mL | Tablet | tablet |
| Compound of Formula I | 10 | Compound of Formula I | 25 |
| Methylcellulose | 5.0 | Microcrystalline Cellulose | 415 |
| Tween 80 | 0.5 | Povidone | 14.0 |
| Benzyl alcohol | 9.0 | Pregelatinized Starch | 43.5 |
| Benzalkonium chloride | 1.0 | Magnesium Stearate | 2.5 |

| | |
|---|---|
| Water for injection to a total volume of 1 mL | 500 |

| | mg/ | | Per | |
|---|---|---|---|---|
| Capsule | capsule | Aerosol | canister | |
| Compound of | 25 | Compound of Formula I | 24 | |
| Formula I | | | | |
| mgLactose Powder | 573.5 | Lecithin, NF Liq. Conc. | 1.2 | mg |
| Magnesium Stearate | 1.5 | Trichlorofluoromethane, | 4.025 | g |
| | 600 | NF | | |
| | | Dichlorodifluoromethane, | 12.15 | g |
| | | NF | | |

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I include, but are not limited to: antipsychotic agents, cognition enhancing agents, anti-migraine agents, anti-asthmatic agents, antiinflammatory agents, anxiolytics, anti-Parkinson's agents, anti-epileptics, anorectic agents, serotonin reuptake inhibitors, other anti-obesity agents, as well as antidiabetic agents, lipid lowering agents, and antihypertensive agents which may be administered separately or in the same pharmaceutical compositions.

The present invention also provides a method for the treatment or prevention of a CB1 receptor modulator mediated disease, which method comprises administration to a patient in need of such treatment or at risk of developing a CB1 receptor modulator mediated disease of an amount of a CB1 receptor modulator and an amount of one or more active ingredients, such that together they give effective relief.

In a further aspect of the present invention, there is provided a pharmaceutical composition comprising a CB1 receptor modulator and one or more active ingredients, together with at least one pharmaceutically acceptable carrier or excipient.

Thus, according to a further aspect of the present invention there is provided the use of a CB1 receptor modulator and one or more active ingredients for the manufacture of a medicament for the treatment or prevention of a CB1 receptor modulator mediated disease. In a further or alternative aspect of the present invention, there is therefore provided a product comprising a CB1 receptor modulator and one or more active ingredients as a combined preparation for simultaneous, separate or sequential use in the treatment or prevention of CB1 receptor modulator mediated disease. Such a combined preparation may be, for example, in the form of a twin pack.

It will be appreciated that for the treatment or prevention of eating disorders, including obesity, bulimia nervosa and compulsive eating disorders, a compound of the present invention may be used in conjunction with other anorectic agents.

The present invention also provides a method for the treatment or prevention of eating disorders, which method comprises administration to a patient in need of such treatment an amount of a compound of the present invention and an amount of an anorectic agent, such that together they give effective relief.

Suitable anorectic agents of use in combination with a compound of the present invention include, but are not limited to, a minorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; and pharmaceutically acceptable salts thereof. A particularly suitable class of anorectic agent are the halogenated amphetamine derivatives, including chlorphentermine, cloforex, clortermine, dexfenfluramine, fenfluramine, picilorex and sibutramine; and pharmaceutically acceptable salts thereof. Particular halogenated amphetamine derivatives of use in combination with a compound of the present invention include: fenfluramine and dexfenfluramine, and pharmaceutically acceptable salts thereof.

The present invention also provides a method for the treatment or prevention of obesity, which method comprises administration to a patient in need of such treatment an amount of a compound of the present invention and an amount of another agent useful in treating obesity and obesity-related conditions, such that together they give effective relief.

Suitable agents of use in combination with a compound of the present invention, include, but are not limited to:

(a) anti-diabetic agents such as (1) PPARγ agonists such as glitazones (e.g. ciglitazone; darglitazone; englitazone; isaglitazone (MCC-555); pioglitazone (ACTOS); rosiglitazone (AVANDIA); troglitazone; rivoglitazone, BRL49653; CLX-0921; 5-BTZD, GW-0207, LG-100641, R483, and LY-300512, and the like and compounds disclosed in WO97/10813, 97/27857, 97/28115, 97/28137, 97/27847, 03/000685, and 03/027112 and SPPARMS (selective PPAR gamma modulators) such as T131 (Amgen), FK614 (Fujisawa), netoglitazone, and metaglidasen; (2) biguanides such as buformin; metformin; and phenformin, and the like; (3) protein tyrosine phosphatase-1B (PTP-1B) inhibitors such as ISIS113715, A-401674, A-364504, IDD-3, IDD 2846, KP-40046, KR61639, MC52445, MC52453, C7, OC-060062, OC-86839, OC29796, TTP-277BC1, and those agents disclosed in WO 04/041799, 04/050646, 02/26707, 02/26743, 04/092146, 03/048140, 04/089918, 03/002569, 04/065387, 04/127570, and US 2004/167183; (4) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (5) meglitinides such as repaglinide, metiglinide (GLUFAST) and nateglinide, and the like; (6) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (7) alpha-amylase inhibitors such as tendamistat, trestatin, and A1-3688, and the like; (8) insulin secreatagogues such as linogliride nateglinide, mitiglinide (GLUFAST), ID1101 A-4166, and the like; (9) fatty acid oxidation inhibitors, such as clomoxir, and etomoxir, and the like; (10) A2 antagonists, such as midaglizole; isaglidole; deriglidole; idazoxan; earoxan; and fluparoxan, and the like; (11) insulin or insulin mimetics, such as biota, LP-100, novarapid, insulin detemir, insulin lispro, insulin glargine, insulin zinc suspension (lente and ultralente); Lys-Pro insulin, GLP-1 (17-36), GLP-1 (73-7) (insulintropin); GLP-1 (7-36)-NH₂) exenatide/Exendin-4, Exenatide LAR, Linaglutide, AVE0010, CJC 1131, BIM51077, CS 872, THO318, BAY-694326, GP010, ALBUGON (GLP-1 fused to albumin), HGX-007 (Epac agonist), S-23521, and compounds disclosed in WO 04/022004, WO 04/37859, and the like; (12) non-thiazolidinediones such as JT-501, and farglitazar (GW-2570/GI-262579), and the like; (13) PPARα/γ dual agonists such as AVE 0847, CLX-0940, GW-1536, GW1929, GW-2433, ICRP-297, L-796449, LBM 642, LR-90, LY510919, MK-0767, ONO 5129, SB 219994, TAK-559, TAK-654, 677954 (GlaxoSmithkline), E-3030 (Eisai), LY510929 (Lilly), AK109 (Asahi), DRF2655 (Dr. Reddy), DRF8351 (Dr. Reddy), MC3002 (Maxocore), TY51501 (ToaEiyo), naveglitazar, muraglitizar, peliglitazar, tesaglitazar (GALIDA), reglitazar (JTT-501), chiglitazar, and those disclosed in WO 99/16758, WO 99/19313, WO 99/20614, WO 99/38850, WO 00/23415, WO 00/23417, WO 00/23445, WO 00/50414, WO 01/00579, WO 01/79150, WO 02/062799, WO 03/033481, WO 03/033450, WO 03/033453; and (14) other insulin sensitizing drugs; (15) VPAC2 receptor agonists; (16) GLK modulators, such as PSN105, RO 281675, RO 274375 and those disclosed in WO 03/015774, WO 03/000262, WO 03/055482, WO 04/046139, WO 04/045614, WO 04/063179, WO 04/063194, WO 04/050645, and the like; (17) retinoid modulators such as those disclosed in WO 03/000249; (18) GSK 3beta/GSK 3 inhibitors such as 4-[2-(2-bromophenyl)-4-(4-fluorophenyl-1H-imidazol-5-yl]pyridine, CT21022, CT20026, CT-98023, SB-216763, SB410111, SB-675236, CP-70949, XD4241 and those compounds disclosed in WO 03/037869, 03/03877, 03/037891, 03/024447, 05/000192, 05/019218 and the like; (19) glycogen phosphorylase (HGLPa) inhibitors, such as AVE 5688, PSN 357, GPi-879, those disclosed in WO 03/037864, WO 03/091213, WO 04/092158, WO 05/013975, WO 05/013981, US 2004/0220229, and JP 2004-196702, and the like; (20) ATP consumption promotors such as those disclosed in WO 03/007990; (21) fixed combinations of PPARγ agonists and metformin such as AVANDAMET; (22) PPAR pan agonists such as GSK 677954; (23) GPR40 (G-protein coupled receptor 40) also called SNORF 55 such as BG 700, and those disclosed in WO 04/041266, 04/022551, 03/099793; (24) GPR119 (also called RUP3; SNORF 25) such as RUP3, HGPRBMY26, PFI 007, SNORF 25; (25) adenosine receptor 2B antagonists such as ATL-618, ATl-802, E3080, and the like; (26) camitine palmitoyl transferase inhibitors such as ST 1327, and ST 1326, and the like; (27) Fructose 1,6-bisphosphohatase inhibitors such as CS-917, MB7803, and the like; (28) glucagon antagonists such as AT77077, BAY 694326, GW 4123X, NN2501, and those disclosed in WO 03/064404, WO 05/00781, US 2004/0209928, US 2004/029943, and the like; (30) glucose-6-phosphase inhibitors; (31) phosphoenolpyruvate carboxykinase (PEPCK) inhibitors; (32) pyruvate dehydrogenase kinase (PDK) activators; (33) RXR agonists such as MC1036, CS00018, JNJ 10166806, and those disclosed in WO 04/089916, U.S. Pat. No. 6,759,546, and the like; (34) SGLT inhibitors such as AVE 2268, KGT 1251, T1095/RWJ 394718; (35) BLX-1002;

(b) lipid lowering agents such as (1) bile acid sequestrants such as, cholestyramine, colesevelem, colestipol, dialkylaminoalkyl derivatives of a cross-linked dextran; Colestid®; LoCholest®; and Questran®, and the like; (2) HMG-CoA reductase inhibitors such as atorvastatin, itavastatin, pitavastatin, fluvastatin, lovastatin, pravastatin, rivastatin, rosuvastatin, simvastatin, rosuvastatin (ZD-4522), and the like, particularly simvastatin; (3) HMG-CoA synthase inhibitors; (4) cholesterol absorption inhibitors such as FMVP4 (Forbes Medi-Tech), KT6-971 (Kotobuki Pharmaceutical), FM-VA12 (Forbes Medi-Tech), FM-VP-24 (Forbes Medi-Tech), stanol esters, beta-sitosterol, sterol glycosides such as tiqueside; and azetidinones such as ezetimibe, and those disclosed in WO 04/005247 and the like; (5) acyl coenzyme A-cholesterol acyl transferase (ACAT) inhibitors such as avasimibe, eflucimibe, pactimibe (KY505), SMP 797 (Sumitomo), SM32504 (Sumitomo), and those disclosed in WO 03/091216, and the like; (6) CETP inhibitors such as JTT 705 (Japan Tobacco), torcetrapib, CP 532,632, BAY63-2149 (Bayer), SC 591, SC 795, and the like; (7) squalene synthetase inhibitors; (8) anti-oxidants such as probucol, and the like; (9) PPARα agonists such as beclofibrate, benzafibrate, ciprofibrate, clofibrate, etofibrate, fenofibrate, gemcabene, and gemfibrozil, GW 7647, BM 170744 (Kowa), LY518674 (Lilly), GW590735 (GlaxoSmithkline), KRP-101 (Kyorin), DRF10945 (Dr. Reddy), NS-2201R1593 (Nippon Shinyaku/Roche, ST1929 (Sigma Tau) MC3001/MC3004 (MaxoCore Pharmaceuticals, gemcabene calcium, other fibric acid derivatives, such as Atromid®, Lopid® and Tricor®, and those disclosed in U.S. Pat. No. 6,548,538, and the like; (10) FXR receptor modulators such as GW 4064 (GlaxoSmithkline), SR 103912, QRX401, LN-6691 (Lion Bioscience), and those disclosed in WO 02/064125, WO 04/045511, and the like; (11) LXR receptor modulators such as GW 3965 (GlaxoSmithkline), T9013137, and XTC0179628 (X-Ceptor Therapeutics/Sanyo), and those disclosed in WO 03/031408, WO 03/063796, WO 04/072041, and the like; (12) lipoprotein synthesis inhibitors such as niacin; (13) renin angiotensin system inhibitors; (14) PPAR δ partial agonists, such as those disclosed in WO 03/024395; (15) bile acid reabsorption inhibitors, such as BARI 1453, SC435, PHA384640, S8921, AZD7706, and the like; and bile acid sequesterants such as colesevelam (WELCHOL/CHOLESTAGEL), (16) PPAR [symbol] agonists such as GW 501516 (Ligand, GSK), GW 590735, GW-0742 (GlaxoSmithkline), T659 (Amgen/Tularik), LY934 (Lilly), NNC610050 (Novo Nordisk) and those disclosed in WO97/28149, WO 01/79197, WO 02/14291, WO 02/46154, WO 02/46176, WO 02/076957, WO 03/016291, WO 03/033493, WO 03/035603, WO 03/072100, WO 03/097607, WO 04/005253, WO 04/007439, and JP10237049, and the like; (17) triglyceride synthesis inhibitors; (18) microsomal triglyceride transport (MTTP) inhibitors, such as implitapide, LAB687, JTT130 (Japan Tobacco), CP346086, and those disclosed in WO 03/072532, and the like; (19) transcription modulators; (20) squalene epoxidase inhibitors; (21) low density lipoprotein (LDL) receptor inducers; (22) platelet aggregation inhibitors; (23) 5-LO or FLAP inhibitors; and (24) niacin receptor agonists including HM74A receptor agonists; (25) PPAR modulators such as those disclosed in WO 01/25181, WO 01/79150, WO 02/79162, WO 02/081428, WO 03/016265, WO 03/033453; (26) niacin-bound chromium, as disclosed in WO 03/039535; (27) substituted acid derivatives disclosed in WO 03/040114; (28) infused HDL such as LUV/ETC-588 (Pfizer), APO-A1 Milano/ETC216 (Pfizer), ETC-642 (Pfizer), ISIS301012, D4F (Bruin Pharma), synthetic trimeric ApoA1, Bioral Apo A1 targeted to foam cells, and the like; (29) IBAT inhibitors such as BAR1143/HMR145A/HMR1453 (Sanofi-Aventis, PHA384640E (Pfizer), S8921 (Shionogi) AZD7806 (AstrZeneca), AK105 (Asah Kasei), and the like; (30) Lp-PLA2 inhibitors such as SB480848 (GlaxoSmithkline), 659032 (GlaxoSmithkline), 677116 (GlaxoSmithkline), and the like; (31) other agents which affect lipic composition including ETC1001/ESP31015 (Pfizer), ESP-55016 (Pfizer), AGI1067 (AtheroGenics), AC3056 (Amylin), AZD4619 (AstrZeneca); and

(c) anti-hypertensive agents such as (1) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, bamidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as candesartan, eprosartan, irbesartan, losartan, pratosartan, tasosartan, telmisartan, valsartan, and EXP-3137, FI6828K, and RNH6270, and the like; (9) α/ [symbol] adrenergic blockers as nipradilol, arotinolol and amosulalol, and the like; (10) alpha 1 blockers, such as terazosin, urapidil, prazosin, bunazosin, trimazosin, doxazosin, naftopidil, indoramin, WHIP 164, and XEN010, and the like; (11) alpha 2 agonists such as lofexidine, tiamenidine, moxonidine, rilmenidine and guanobenz, and the like; (12) aldosterone inhibitors, and the like; (13) angiopoietin-2-binding agents such as those disclosed in WO 03/030833; and

(d) anti-obesity agents, such as (1) 5HT (serotonin) transporter inhibitors, such as paroxetine, fluoxetine, fenfluramine, fluvoxamine, sertraline, and imipramine, and those disclosed in WO 03/00663, as well as serotonin/noradrenaline re uptake inhibitors such as sibutramine (MERIDIA/REDUCTIL) and dopamine uptake inhibitor/Norepenephrine uptake inhibitors such as radafaxine hydrochloride, 353162 (GlaxoSmithkline), and the like; (2) NE (norepinephrine) transporter inhibitors, such as GW 320659, despiramine, talsupram, and nomifensine; (3) CB1 (caimabinoid-1 receptor) antagonist/inverse agonists, such as rimonabant (ACCOMPLIA Sanofi Synthelabo), SR-147778 (Sanofi Synthelabo), AVE1625 (Sanofi-Aventis), BAY 65-2520 (Bayer), SLV 319 (Solvay), SLV326 (Solvay), CP945598 (Pfizer), E-6776 (Esteve), O1691 (Organix), ORG14481 (Organon), VER24343 (Vemalis), NESS0327 (Univ of Sassari/Univ of Cagliari), and those disclosed in U.S. Pat. Nos. 4,973,587, 5,013,837, 5,081,122, 5,112,820, 5,292,736, 5,532,237, 5,624,941, 6,028,084, and 6,509367; and WO 96/33159, WO97/29079, WO98/31227, WO 98/33765, WO98/37061, WO98/41519, WO98/43635, WO98/43636, WO99/02499, WO00/10967, WO00/10968, WO 01/09120, WO 01/58869, WO 01/64632, WO 01/64633, WO 01/64634, WO 01/70700, WO 01/96330, WO 02/076949, WO 03/006007, WO 03/007887, WO 03/020217, WO 03/026647, WO 03/026648, WO 03/027069, WO 03/027076, WO 03/027114, WO 03/037332, WO 03/040107, WO 04/096763, WO 04/111039, WO 04/111033, WO 04/111034, WO 04/111038, WO 04/013120, WO 05/000301, WO 05/016286, WO 05/066126 and EP-658546 and the like; (4) ghrelin agonists/antagonists, such as BVT81-97 (BioVitrum), RC1291 (Rejuvenon), SRD-04677 (Sumitomo), unacylated ghrelin (TheraTechnologies), and those disclosed in WO 01/87335, WO 02/08250, WO 05/012331, and the like; (5) H3 (histamine 113) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, and those disclosed in WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO 03/024928 and WO 03/024929; (6) melanin-concentrating hormone 1 receptor (MCH1R) antagonists, such as T-226296 (Takeda), T71 (Takeda/Amgen), AMGN-608450, AMGN-503796 (Amgen), 856464 (GlaxoSmithkline), A224940 (Abbott), A798 (Abbott), ATC0175/AR224349 (Arena Pharmaceuticals), GW803430 (GlaxoSmithkine), NBI-1A (Neurocrine Biosciences), NGX-1 (Neurogen), SNP-7941 (Synaptic), SNAP9847 (Synaptic), T-226293 (Schering Plough), TPI-1361-17 (Saitama Medical School/University of California Irvine), and those disclosed WO 01/21169, WO 01/82925, WO 01/87834, WO 02/051809, WO 02/06245, WO 02/076929, WO 02/076947, WO 02/04433, WO 02/51809, WO 02/083134, WO 02/094799, WO 03/004027, WO 03/13574, WO 03/15769, WO 03/028641, WO 03/035624, WO 03/033476, WO 03/033480, WO 04/004611, WO 04/004726, WO 04/011438, WO 04/028459, WO 04/034702, WO 04/039764, WO 04/052848, WO 04/087680; and Japanese Patent Application Nos. JP 13226269, JP 1437059, JP2004315511, and the like; (7) MCH2R (melanin concentrating hormone 2R) agonist/antagonists; (8) NPY1 (neuropeptide Y Y1) antagonists, such as BMS205749, BIBP3226, J-115814, BIBO 3304, LY-357897, CP-671906, and GI-264879A; and those disclosed in U.S. Pat. No. 6,001,836; and WO 96/14307, WO 01/23387, WO 99/51600, WO 01/85690, WO 01/85098, WO 01/85173, and WO 01/89528; (9) NPY5 (neuropeptide Y Y5) antagonists, such as 152,804, S2367 (Shionogi), E-6999 (Esteve), GW-569180A, GW-594884A (GlaxoSmithkline), GW-587081X, GW-548118X; FR 235,208; FR226928, FR240662, FR252384; 1229U91, GI-264879A, CGP71683A, C-75 (Fasgen) LY-377897, LY366377, PD-160170, SR-120562A, SR-120819A, S2367 (Shionogi), JCF-104, and H409/22; and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683; and EP-01010691, EP-01044970, and FR252384; and PCT Publication Nos. WO 97/19682, WO 97/20820, WO 97/20821, WO 97/20822, WO 97/20823, WO 98/27063, WO 00/107409, WO 00/185714, WO 00/185730, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/20488, WO 02/22592, WO 02/48152, WO 02/49648, WO 02/051806, WO 02/094789, WO 03/009845, WO 03/014083, WO 03/022849, WO 03/028726, WO 05/014592, WO 05/01493; and Norman et al., J. Med. Chem. 43:4288-4312 (2000); (10) leptin, such as recombinant human leptin (PEG-OB, Hoffman La Roche) and recombinant methionyl human leptin (Amgen); (11) leptin derivatives, such as those disclosed in U.S. Pat. Nos. 5,552,524; 5,552,523; 5,552,522; 5,521,283; and WO 96/23513; WO 96/23514; WO 96/23515; WO 96/23516; WO 96/23517; WO 96/23518; WO 96/23519; and WO 96/23520; (12) opioid antagonists, such as nalmefene (Revex®), 3-methoxynaltrexone, naloxone, and naltrexone; and those disclosed in WO 00/21509; (13) orexin antagonists, such as SB-334867-A (GlaxoSmithkline); and those disclosed in WO 01/96302, 01/68609, 02/44172, 02/51232, 02/51838, 02/089800, 02/090355, 03/023561, 03/032991, 03/037847, 04/004733, 04/026866, 04/041791, 04/085403, and the like; (14) BRS3 (bombesin receptor subtype 3) agonists; (15) CCK-A (cholecystokinin-A) agonists, such as AR-R 15849, GI 181771, JMV-180, A-71378, A-71623, PD170292, PD 149164, SR146131, SR125180, butabindide, and those disclosed in U.S. Pat. No. 5,739,106; (16) CNTF (ciliary neurotrophic factors), such as GI-181771 (Glaxo-SmithKline); SR146131 (Sanofi Synthelabo); butabindide; and PD170,292, PD 149164 (Pfizer); (17) CNTF derivatives, such as axokine (Regeneron); and those disclosed in WO 94/09134, WO 98/22128, and WO 99/43813; (18) GHS (growth hormone secretagogue receptor) agonists, such as NN703, hexarelin, MK-0677, SM-130686, CP-424,391, L-692,429 and L-163,255, and those disclosed in U.S. Pat. No. 6,358,951, U.S. Patent Application Nos. 2002/049196 and 2002/022637; and WO 01/56592, and WO 02/32888; (19) 5HT2c (serotonin receptor 2c) agonists, such as APD3546/AR10A (Arena Pharmaceuticals), ATH88651 (Athersys), ATH88740 (Athersys), BVT933 (Biovitrum/GSK), DPCA37215 (BMS), 110264; LY448100 (Lilly), PNU 22394; WAY 470 (Wyeth), WAY629 (Wyeth), WAY161503 (Biovitrum), R-1065, VR1065 (Vernalis/Roche) YM 348; and those disclosed in U.S. Pat. No. 3,914,250; and PCT Publications 01/66548, 02/36596, 02/48124, 02/10169, 02/44152; 02/51844, 02/40456, 02/40457, 03/057698, 05/000849, and the like; (20) Mc3r (melanocortin 3 receptor) agonists; (21) Mc4r (melanocortin 4 receptor) agonists, such as CHIR86036 (Chiron), CHIR915 (Chiron); ME-10142 (Melacure), ME-10145 (Melacure), HS-131 (Melacure), NBI72432 (Neurocrine Biosciences), NNC 70-619 (Novo Nordisk), TTP2435 (Transtech) and those disclosed in PCT Publications WO 99/64002, 00/74679, 01/991752, 01/0125192, 01/52880, 01/74844, 01/70708, 01/70337, 01/91752, 01/010842, 02/059095, 02/059107, 02/059108, 02/059117, 02/062766, 02/069095, 02/12166, 02/11715, 02/12178, 02/15909, 02/38544, 02/068387, 02/068388, 02/067869, 02/081430, 03/06604, 03/007949, 03/009847, 03/009850, 03/013509, 03/031410, 03/094918, 04/028453, 04/048345, 04/050610, 04/075823, 04/083208, 04/089951, 05/000339, and EP 1460069, and US 2005049269, and JP2005042839, and the like; (22) monoamine reuptake inhibitors, such as sibutratmine (Meridia®/Reductil®) and salts thereof, and those compounds disclosed in U.S. Pat. Nos. 4,746,680, 4,806,570, and 5,436,272, and U.S. Patent Publication No. 2002/0006964, and WO 01/27068, and WO 01/62341; (23) serotonin reuptake inhibitors, such as dexfenfluramine, fluoxetine, and those in U.S. Pat. No. 6,365,633, and WO 01/27060, and WO 01/162341; (24) GLP-1 (glucagon-like peptide 1) agonists; (25) Topiramate (Topimax®); (26) phytopharm compound 57 (CP 644,673); (27) ACC2 (acetyl-CoA carboxylase-2) inhibitors; (28) β3 (beta adrenergic receptor 3) agonists, such as rafebergron/AD9677/TAK677 (Dainippon/Takeda), CL-316,243, SB 418790, BRL-37344, L-796568, BMS-196085, BRL-35135A, CGP12177A, BTA-243, GRC1087 (Glenmark Pharmaceuticals) GW 427353 (solabegron hydrochloride), Trecadrine, Zeneca D7114, N-5984 (Nisshin Kyorin), LY-377604 (Lilly), KT07924 (Kissei), SR 59119A, and those disclosed in U.S. Pat. No. 5,705,515, U.S. Pat. No. 5,451,677; and WO94/18161, WO95/29159, WO97/46556, WO98/04526 WO98/32753, WO 01/74782, WO 02/32897, WO 03/014113, WO 03/016276, WO 03/016307, WO 03/024948, WO 03/024953, WO 03/037881, WO 04/108674, and the like; (29) DGAT1 (diacylglycerol acyltransferase 1) inhibitors; (30) DGAT2 (diacylglycerol acyltransferase 2) inhibitors; (31) FAS (fatty acid synthase) inhibitors, such as Cerulenin and C75; (32) PDE (phosphodiesterase) inhibitors, such as theophylline, pentoxifylline, zaprinast, sildenafil, aminone, milrinone, cilostamide, rolipram, and cilomilast, as well as those described in WO 03/037432, WO 03/037899; (33) thyroid hormone β agonists, such as KB-2611 (KaroBioBMS), and those disclosed in WO 02/15845; and Japanese Patent Application No. JP 2000256190; (34) UCP-1 (uncoupling protein 1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), and retinoic acid; and those disclosed in WO 99/00123; (35) acyl-estrogens, such as oleoyl-estrone, disclosed in del Mar-Grasa, M. et al., Obesity Research, 9:202-9 (2001); (36) glucocorticoid receptor antagonists, such as CP472555 (Pfizer), KB 3305, and those disclosed in WO 04/000869, WO 04/075864, and the like; (37) 11β HSD-1 (11-beta hydroxy steroid dehydrogenase type 1) inhibitors, such as BVT 3498 (AMG 331), BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO 01/90091, 01/90090, 01/90092, 02/072084, 04/011410, 04/033427, 04/041264, 04/027047, 04/056744, 04/065351, 04/089415, 04/037251, and the like; (38) SCD-1 (stearoyl-CoA desaturase-1) inhibitors; (39) dipeptidyl peptidase IV (DPP-4) inhibitors, such as isoleucine thiazolidide, valine pyrrolidide, sitagliptin, saxagliptin, NVP-DPP728, LAF237 (vildagliptin), P93/01, TSL 225, TMC-2A/2B/2C, FE 999011, P9310/K364, VIP 0177, SDZ 274-444, GSK 823093, E 3024, SYR 322, TS021, SSR 162369, GRC 8200, K579, NN7201, CR 14023, PHX 1004, PHX 1149, PT-630, SK-0403; and the compounds disclosed in WO 02/083128, WO 02/062764, WO 02/14271, WO 03/000180, WO 03/000181, WO 03/000250, WO 03/002530, WO 03/002531, WO 03/002553, WO 03/002593, WO 03/004498, WO 03/004496, WO 03/005766, WO 03/017936, WO 03/024942, WO 03/024965, WO 03/033524, WO 03/055881, WO 03/057144, WO 03/037327, WO 04/041795, WO 04/071454, WO 04/0214870, WO 04/041273, WO 04/041820, WO 04/050658, WO 04/046106, WO 04/067509, WO 04/048532, WO 04/099185, WO 04/108730, WO 05/009956, WO 04/09806, WO 05/023762, US 2005/043292, and EP 1 258 476; (40) lipase inhibitors, such as tetrahydrolipstatin (orlistat/XENICAL), ATL962 (Alizyme/Takeda), GT389255 (Genzyme/Peptimmune)Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC 80267, and those disclosed in WO 01/77094, WO 04/111004, and U.S. Pat. Nos. 4,598,089, 4,452,813, 5,512,565, 5,391,571, 5,602,151, 4,405,644, 4,189,438, and 4,242,453, and the like; (41) fatty acid transporter inhibitors; (42) dicarboxylate transporter inhibitors; (43) glucose transporter inhibitors; and (44) phosphate transporter inhibitors; (45) anorectic bicyclic compounds such as 1426 (Aventis) and 1954 (Aventis), and the compounds disclosed in WO 00/18749, WO 01/32638, WO 01/62746, WO 01/62747, and WO 03/015769; (46) peptide YY and PYY agonists such as PYY336 (Nastech/Merck), AC162352 (IC Innovations/Curis/Amylin), TM30335/TM30338 (7™ Pharma), PYY336 (Emisphere Tehcnologies), pegylated peptide YY3-36, those disclosed in WO 03/026591, 04/089279, and the like; (47) lipid metabolism modulators such as maslinic acid, erythrodiol, ursolic acid uvaol, betulinic acid, betulin, and the like and compounds disclosed in WO 03/011267; (48) transcription factor modulators such as those disclosed in WO 03/026576; (49) Mc5r (melanocortin 5 receptor) modulators, such as those disclosed in WO 97/19952, WO 00/15826, WO 00/15790, US 20030092041, and the like; (50) Brain derived neutotropic factor (BDNF), (51) Mc1r (melanocortin 1 receptor modulators such as LK-184 (Proctor & Gamble), and the like; (52) 5HT6 antagonists such as BVT74316 (BioVitrum), BVT5182c (BioVitrum), E-6795 (Esteve), E-6814 (Esteve), SB399885 (GlaxoSmithkline), SB271046 (GlaxoSmithkline), RO-046790 (Roche), and the like; (53) fatty acid transport protein 4 (FATP4); (54) acetyl-CoA carboxylase (ACC) inhibitors such as CP640186, CP610431, CP640188 (Pfizer); (55) C-terminal growth hormone fragments such as AOD9604 (Monash Univ/Metabolic Pharmaceuticals), and the like; (56) oxyntomodulin; (57) neuropeptide FF receptor antagonists such as those disclosed in WO 04/083218, and the like; (58) amylin agonists such as Symlin/pramlintide/AC137 (Amylin); (59) Hoodia and trichocaulon extracts; (60) BVT74713 and other gut lipid appetite suppressants; (61) dopamine agonists such as bupropion (WELLBUTRIN/GlaxoSmithkline); (62) zonisamide (ZONEGRAN/Dainippon/Elan), and the like.

Specific NPY5 antagonists of use in combination with a compound of the present invention include: 3-oxo-N-(5-phenyl-2-pyrazinyl)-spiro[isobenzofuran-1(3H), 4′-piperidine]-1′-carboxamide, 3-oxo-N-(7-trifluoromethylpyrido[3,2-b]pyridin-2-yl)spiro-[isobenzofuran-1(3H), 4′-piperidine]-1′-carboxamide, N-[5-(3-fluorophenyl)-2-pyrimidinyl]-3-oxospiro-[isobenzofuran-1(3H), 4′-piperidine]-1′-carboxamide, trans-3′-oxo-N-(5-phenyl-2-pyrimidinyl)spiro[cyclohexane-1,1′(3′H)-isobenzofuran]-4-carboxamide, trans-3′-oxo-N-[1-(3-quinolyl)-4-imidazolyl]spiro[cyclohexane-1,1′(3′H)-isobenzofuran]-4-carboxamide, trans-3-oxo-N-(5-phenyl-2-pyrazinyl)spiro[4-azaiso-benzofuran-1(3H), 1′-cyclohexane]-4′-carboxamide, trans-N-[5-(3-fluorophenyl)-2-pyrimidinyl]-3-oxospiro[5-azaisobenzofuran-1(3H), 1′-cyclohexane]-4′-carboxamide, trans-N-[5-(2-fluorophenyl)-2-pyrimidinyl]-3-oxospiro[5-azaisobenzofuran-1(3H), 1′-cyclohexane]-4′-carboxamide, trans-N-[1-(3,5-difluorophenyl)-4-imidazolyl]-3-oxospiro[7-azaisobenzofuran-1(3H), 1′-cyclohexane]-4′-carboxamide, trans-3-oxo-N-(1-phenyl-4-pyrazolypspiro[4-azaisobenzofuran-1(3H), 1′-cyclohexane]-4′-carboxamide, trans-N-[1-(2-fluorophenyl)-3-pyrazolyl]-3-oxospiro[6-azaisobenzofuran-1(3H), 1′-cyclohexane]-4′-carboxamide, trans-3-oxo-N-(1-phenyl-3-pyrazolypspiro[6-azaisobenzofuran-1(3H), 1′-cyclohexane]-4′-carboxamide, trans-3-oxo-N-(2-phenyl-1,2,3-triazol-4-yl)spiro[6-azaisobenzofuran-1(3H), 1′-cyclohexane]-4′-carboxamide, and pharmaceutically acceptable salts and esters thereof.

Specific DP-IV inhibitors of use in combination with a compound of the present invention are selected from 7-[(3R)-3-amino-4-(2,4,5-trifluorophenyl)butanoyl]-3-(trifluoromethyl)-5,6,7,8-tetrahydro-1,2,4-triazolo[4,3-a]pyrazine. In particular, the compound of formula I is favorably combined with 7-[(3R)-3-amino-4-(2,4,5-trifluorophenyl)butanoyl]-3-(trifluoromethyl)-5,6,7,8-tetrahydro-1,2,4-triazolo[4,3-a]pyrazine, and pharmaceutically acceptable salts thereof.

“Obesity” is a condition in which there is an excess of body fat. The operational definition of obesity is based on the Body Mass Index (BMI), calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk for obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asian countries, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds of the present invention to reduce or maintain the body weight of an obese subject. One outcome of treatment may be reducing the body weight of an obese subject relative to that subject's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds of the present invention to reduce or maintain the body weight of a subject at risk of obesity. One outcome of prevention may be reducing the body weight of a subject at risk of obesity relative to that subject's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compounds of the present invention are also useful for reducing the risk of secondary outcomes of obesity, such as reducing the risk of left ventricular hypertrophy.

The compounds of formula I are also useful for treating or preventing obesity and obesity-related disorders in cats and dogs. As such, the term “mammal” includes companion animals such as cats and dogs.

The term “diabetes,” as used herein, includes both insulin-dependent diabetes mellitus (IDDM, also known as type I diabetes) and non-insulin-dependent diabetes mellitus (NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compounds of the present invention are useful for treating both Type I and Type II diabetes. The compounds are especially effective for treating Type II diabetes. The compounds of the present invention are also useful for treating and/or preventing gestational diabetes mellitus.

It will be appreciated that for the treatment or prevention of migraine, a compound of the present invention may be used in conjunction with other anti-migraine agents, such as ergotamines or 5-HT₁ agonists, especially sumatriptan, naratriptan, zolmatriptan or rizatriptan.

It will be appreciated that for the treatment of depression or anxiety, a compound of the present invention may be used in conjunction with other anti-depressant or anti-anxiety agents.

Suitable classes of anti-depressant agents include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists and atypical anti-depressants.

Suitable norepinephrine reuptake inhibitors include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Suitable selective serotonin reuptake inhibitors include: fluoxetine, fluvoxamine, paroxetine, imipramine and sertraline, and pharmaceutically acceptable salts thereof.

Suitable monoamine oxidase inhibitors include: isocarboxazid, phenelzine, tranylcypromine and selegiline, and pharmaceutically acceptable salts thereof.

Suitable reversible inhibitors of monoamine oxidase include: moclobemide, and pharmaceutically acceptable salts thereof.

Suitable serotonin and noradrenaline reuptake inhibitors of use in the present invention include: venlafaxine, and pharmaceutically acceptable salts thereof.

Suitable CRF antagonists include those compounds described in International Patent Specification Nos. WO 94/13643, 94/13644, 94/13661, 94/13676 and 94/13677. Still further, neurokinin-1 (NK-1) receptor antagonists may be favorably employed with the CB1 receptor modulators of the present invention. NK-1 receptor antagonists of use in the present invention are fully described in the art. Specific neurokinin-1 receptor antagonists of use in the present invention include: W-(2R3R,2S3S)—N-{[2-cyclopropoxy-5-(trifluoromethoxy)-phenyl]methyl}-2-phenylpiperidin-3-amine; 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine; aperpitant; CJ17493; GW597599; GW679769; R673; RO67319; R1124; R1204; SSR146977; SSR240600; T-2328; and T2763; or a pharmaceutically acceptable salts thereof.

Suitable atypical anti-depressants include: bupropion, lithium, nefazodone, trazodone and viloxazine, and pharmaceutically acceptable salts thereof.

Suitable classes of anti-anxiety agents include benzodiazepines and 5-HT_{1A} agonists or antagonists, especially 5-HT_{1A} partial agonists, and corticotropin releasing factor (CRF) antagonists. Suitable benzodiazepines include: alprazolam, chlordiazepoxide, clonazepam, chlorazepate, diazepam, halazepam, lorazepam, oxazepam and prazepam, and pharmaceutically acceptable salts thereof. Suitable 5-HT_{1A} receptor agonists or antagonists include, in particular, the 5-HT_{1A} receptor partial agonists buspirone, flesinoxan, gepirone and ipsapirone, and pharmaceutically acceptable salts thereof. Suitable corticotropin releasing factor (CRF) antagonists include those previously discussed herein.

As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above.

In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances. It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

In particular, compounds of structural formula I are useful for aiding in stopping consumption of tobacco and are useful in treating nicotine dependence and nicotine withdrawal. The compounds of formula I produce in consumers of nicotine, such as tobacco smokers, a total or partial abstinence from smoking. Further, withdrawal symptoms are lessened and the weight gain that generally accompanies quitting tobacco consumption is reduced or nonexistent. For smoking cessation, the compound of form I may be used in combination with a nicotine agonist or a partial nicotine agonist, including varenicline and selective alpha-4 beta 2 nicotinic partial agonists such as SSR 591813, or a monoamine oxidase inhibitor (MAOI), or another active ingredient demonstrating efficacy in aiding cessation of tobacco consumption; for example, an antidepressant such as bupropion, doxepine, ornortriptyline; or an anxiolytic such as buspirone or clonidine.

It will be appreciated that a combination of a conventional antipsychotic drug with a CBI receptor modulator may provide an enhanced effect in the treatment of mania. Such a combination would be expected to provide for a rapid onset of action to treat a manic episode thereby enabling prescription on an “as needed basis”. Furthermore, such a combination may enable a lower dose of the antispychotic agent to be used without compromising the efficacy of the antipsychotic agent, thereby minimizing the risk of adverse side-effects. A yet further advantage of such a combination is that, due to the action of the CB 1 receptor modulator, adverse side-effects caused by the antipsychotic agent such as acute dystonias, dyskinesias, akathesia and tremor may be reduced or prevented.

Thus, according to a further aspect of the present invention there is provided the use of a CB1 receptor modulator and an antipsychotic agent for the manufacture of a medicament for the treatment or prevention of mania. The present invention also provides a method for the treatment or prevention of mania, which method comprises administration to a patient in need of such treatment or at risk of developing mania of an amount of a CB1 receptor modulator and an amount of an antipsychotic agent, such that together they give effective relief. In a further aspect of the present invention, there is provided a pharmaceutical composition comprising a CB1 receptor modulator and an antipsychotic agent, together with at least one pharmaceutically acceptable carrier or excipient, wherein the CB1 receptor modulator and the antipsychotic agent may be present as a combined preparation for simultaneous, separate or sequential use for the treatment or prevention of mania. Such combined preparations may be, for example, in the form of a twin pack. In a further or alternative aspect of the present invention, there is therefore provided a product comprising a CB1 receptor modulator and an antipsychotic agent as a combined preparation for simultaneous, separate or sequential use in the treatment or prevention of mania.

Included within the scope of the present invention is the use of CB1 receptor modulators in combination with an antipsychotic agent in the treatment or prevention of hypomania.

It will be appreciated that a combination of a conventional antipsychotic drug with a CB1 receptor modulator may provide an enhanced effect in the treatment of schizophrenic disorders. Such a combination would be expected to provide for a rapid onset of action to treat schizophrenic symptoms thereby enabling prescription on an “as needed basis”. Furthermore, such a combination may enable a lower dose of the CNS agent to be used without compromising the efficacy of the antipsychotic agent, thereby minimizing the risk of adverse side-effects. A yet further advantage of such a combination is that, due to the action of the CB1 receptor modulator, adverse side-effects caused by the antipsychotic agent such as acute dystonias, dyskinesias, akathesia and tremor may be reduced or prevented.

As used herein, the term “schizophrenic disorders” includes paranoid, disorganized, catatonic, undifferentiated and residual schizophrenia; schizophreniform disorder; schizoaffective disorder; delusional disorder; brief psychotic disorder; shared psychotic disorder; substance-induced psychotic disorder; and psychotic disorder not otherwise specified. Other conditions commonly associated with schizophrenic disorders include self-injurious behavior (e.g. Lesch-Nyhan syndrome) and suicidal gestures.

Suitable antipsychotic agents of use in combination with a CB1 receptor modulator include the phenothiazine, thioxanthene, heterocyclic dibenzazepine, butyrophenone, diphenylbutylpiperidine and indolone classes of antipsychotic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. Suitable examples of dibenzazepines include clozapine and olanzapine. An example of a butyrophenone is haloperidol. An example of a diphenylbutylpiperidine is pimozide. An example of an indolone is molindolone. Other antipsychotic agents include loxapine, sulpiride and risperidone. It will be appreciated that the antipsychotic agents when used in combination with a CB1 receptor modulator may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, olanzapine, haloperidol, pimozide and risperidone are commonly used in a non-salt form.

Other classes of antipsychotic agent of use in combination with a CB1 receptor modulator include dopamine receptor antagonists, especially D2, D3 and D4 dopamine receptor antagonists, and muscarinic m1 receptor agonists. An example of a D3 dopamine receptor antagonist is the compound PNU-99194A. An example of a D4 dopamine receptor antagonist is PNU-101387. An example of a muscarinic m1 receptor agonist is xanomeline.

Another class of antipsychotic agent of use in combination with a CB1 receptor modulator is the 5-HT_{2A} receptor antagonists, examples of which include MDL100907 and fananserin. Also of use in combination with a CB1 receptor modulator are the serotonin dopamine antagonists (SDAs) which are believed to combine 5-HT_{2A} and dopamine receptor antagonist activity, examples of which include olanzapine and ziperasidone.

Still further, NK-1 receptor antagonists may be favorably employed with the CB1 receptor modulators of the present invention. Preferred NK-1 receptor antagonists for use in the present invention are selected from the classes of compounds described previously.

It will be appreciated that a combination of a conventional anti-asthmatic drug with a CB1 receptor modulator may provide an enhanced effect in the treatment of asthma, and may be used for the treatment or prevention of asthma, which method comprises administration to a patient in need of such treatment an amount of a compound of the present invention and an amount of an anti-asthmatic agent, such that together they give effective relief.

Suitable anti-asthmatic agents of use in combination with a compound of the present invention include, but are not limited to: (a) VLA-4 antagonists such as natalizumab and the compounds described in U.S. Pat. No. 5,510,332, WO97/03094, WO97/02289, WO96/40781, WO96/22966, WO96/20216, WO96/01644, WO96/06108, WO95/15973 and WO96/31206; (b) steroids and corticosteroids such as beclomethasone, methylprednisolone, betamethasone, prednisone, dexamethasone, and hydrocortisone; (c) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (d) non-steroidal anti-asthmatics including β2-agonists (such as terbutaline, metaproterenol, fenoterol, isoetharine, albuterol, bitolterol, salmeterol, epinephrine, and pirbuterol), theophylline, cromolyn sodium, atropine, ipratropium bromide, leukotriene antagonists (such as zafirlukast, montelukast, pranlukast, iralukast, pobilukast, and SKB-106,203), and leukotriene biosynthesis inhibitors (such as zileuton and BAY-1005); (e) anti-cholinergic agents including muscarinic antagonists (such as ipratropium bromide and atropine); and (f) antagonists of the chemokine receptors, especially CCR-3; and pharmaceutically acceptable salts thereof.

It will be appreciated that a combination of a conventional anti-constipation drug with a CB1 receptor modulator may provide an enhanced effect in the treatment of constipation or chronic intestinal pseudo-obstruction, and for use for the manufacture of a medicament for the treatment or prevention of constipation or chronic intestinal pseudo-obstruction.

The present invention also provides a method for the treatment or prevention of constipation, which method comprises administration to a patient in need of such treatment an amount of a compound of the present invention and an amount of an anti-constipation agent, such that together they give effective relief.

Suitable anti-constipation agents of use in combination with a compound of the present invention include, but are not limited to, osmotic agents, laxatives and detergent laxatives (or wetting agents), bulking agents, and stimulants; and pharmaceutically acceptable salts thereof. A particularly suitable class of osmotic agents include, but are not limited to sorbitol, lactulose, polyethylene glycol, magnesium, phosphate, and sulfate; and pharmaceutically acceptable salts thereof. A particularly suitable class of laxatives and detergent laxatives, include, but are not limited to, magnesium, and docusate sodium; and pharmaceutically acceptable salts thereof. A particularly suitable class of bulking agents include, but are not limited to, psyllium, methylcellulose, and calcium polycarbophil; and pharmaceutically acceptable salts thereof. A particularly suitable class of stimulants include, but are not limited to, anthroquinones, and phenolphthalein; and pharmaceutically acceptable salts thereof.

It will be appreciated that a combination of a conventional anti-cirrhosis drug with a CB1 receptor modulator may provide an enhanced effect in the treatment or prevention of cirrhosis of the liver, and for use for the manufacture of a medicament for the treatment or prevention of cirrhosis of the liver, as well as non-alcoholic fatty liver disease (NAFLD) and non-alcoholic steatohepatitis (NASH).

The present invention also provides a method for the treatment or prevention of cirrhosis of the liver, which method comprises administration to a patient in need of such treatment an amount of a compound of the present invention and an anti-cirrhosis agent, such that together they give effective relief.

Suitable anti-cirrhosis agents of use in combination with a compound of the present invention include, but are not limited to, corticosteroids, penicillamine, colchicine, interferon-γ, 2-oxoglutarate analogs, prostaglandin analogs, and other anti-inflammatory drugs and antimetabolites such as azathioprine, methotrexate, leflunamide, indomethacin, naproxen, and 6-mercaptopurine; and pharmaceutically acceptable salts thereof.

The method of treatment of this invention comprises a method of modulating the CB1 receptor and treating CB1 receptor mediated diseases by administering to a patient in need of such treatment a non-toxic therapeutically effective amount of a compound of this invention that selectively antagonizes the CB1 receptor in preference to the other CB or G-protein coupled receptors.

The term “therapeutically effective amount” means the amount the compound of structural formula I that will elicit the biological or medical response of a tissue, system, animal or human that is being sought by the researcher, veterinarian, medical doctor or other clinician, which includes alleviation of the symptoms of the disorder being treated. The novel methods of treatment of this invention are for disorders known to those skilled in the art. The term “mammal” includes humans, and companion animals such as dogs and cats.

The weight ratio of the compound of the Formula I to the second active ingredient may be varied and will depend upon the effective dose of each ingredient. Generally, an effective dose of each will be used. Thus, for example, when a compound of the Formula I is combined with a β-3 agonist the weight ratio of the compound of the Formula I to theft-3 agonist will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

Abbreviations used in the following Schemes and Examples: aq.: aqueous; API-ES: atmospheric pressure ionization-electrospray (mass spectrum term); AcCN: acetonitrile; DEAD: diethyl azodicarboxylate; DMAP: 4-dimethylaminopyridine; DMF: dimethylformamide; DMSO: dimethylsulfoxide; EDC: 1-ethyl-3-(3-dimethylaminopropyl)-carbodiimide hydrochloride; EPA: ethylene polyacrylamide (a plastic); EtOAc: ethyl acetate; g: gram; h: hours; Hex: hexane; HOBt: 1-hydroxybenzotriazole; HPLC: high pressure liquid chromatography; HPLC/MS: high pressure liquid chromatography/mass spectrum; in vacuo: rotoevaporation; IPAC or IPAc: isopropyl acetate; KIIMDS: potassium hexamethyldisilazide; LC: Liquid chromatography; LC/MS, LC-MS: liquid chromatography-mass spectrum; LDA: lithium diisopropyl amide; M: molar; Me: methyl; MeOH: methanol; MHz: megahertz; min: minute; mL: milliliter; mmol: millimole; MS or ms: mass spectrum; N: normal; NaHMDS: sodium hexamethyldisilazide; NMR: nuclear magnetic resonance; NIVIM: N-methylmorpholine; PyBOP: (benzotriazol-1-yloxy)tripyrrolidinophosphonium hex afluorophosphate; Rₜ: retention time; rt or RT: room temperature; TFA: trifluoroacetic acid; THF: tetrahydrofuran; TLC: thin layer chromatography

Compounds may be prepared by procedures illustrated in the accompanying schemes.

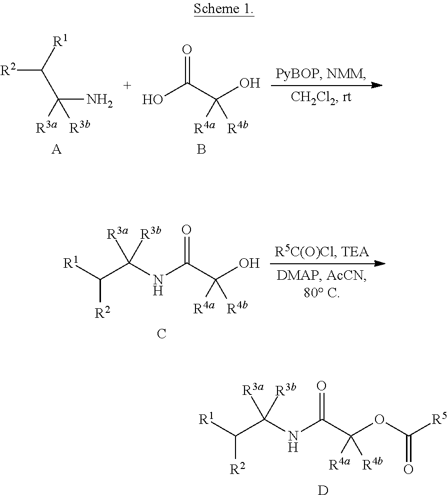

In Scheme 1, an appropriately substituted amine A is reacted with an α-hydroxy carboxylic acid B in the presence of PyBOP and N-methylmorpholine to afford the amide C. The alcohol C is reacted with an acid halide in the presence of an amine base and DMAP to yield the ester D.

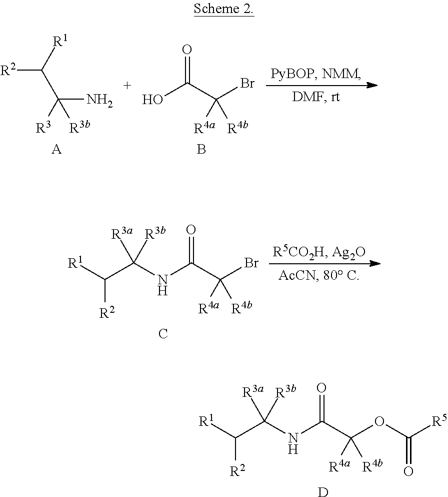

In Scheme 2, an alternative synthesis is outlined. an appropriately substituted amine A is reacted with an α-halo carboxylic acid B in the presence of PyBOP and N-methylmorpholine to afford the amide C. The halide C is reacted with a carboxylic acid in the presence of silver oxide to yield the ester D.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### General Chemical Procedures.

The LC/MS analyses were preformed using a MICROMASS ZMD mass spectrometer coupled to an AGILENT 1100 Series HPLC utilizing a YMC ODS-A 4.6×50 mm column eluting at 2.5 mL/min with a solvent gradient of 10 to 95% B over 4.5 min, followed by 0.5 min at 95% B: solvent A=0.06% TFA in water; solvent B=0.05% TFA in acetonitrile. ¹H-NMR spectra were obtained on a 500 MHz VARIAN Spectrometer in CDCl₃ or CD₃OD as indicated and chemical shifts are reported as δ using the solvent peak as reference and coupling constants are reported in hertz (Hz).

### Reference Example 1

### N-[2,3-Bis(4-chlorophenyl)-1-methylpropyl]-amine hydrochloride

The preparation of the two diastereomers (alpha and beta) of N-[2,3-bis(4-chlorophenyl)-1-methylpropyl]-amine hydrochloride salt has been disclosed (Schultz, E. M, et al. J. Med. Chem. 1967, 10, 717). Diastereomer α: LC-MS: calculated for C₁₆H₁₇Cl₂N 293, observed m/e 294 (M+H)⁺ (Rₜ 2.5 min). Diastereomer β: LC-MS: calculated for C₁₆H₁₇Cl₂N 293, observed m/e 294 (M+H)⁺ (Rₜ 2.2 min).

The amines of Reference Examples 2-9 were prepared by the same procedures described in Reference Example 1:

### Reference Example 2

### 2-Amino-3,4-diphenylbutane hydrochloride salt

Diastereomer α: LC-MS: calculated for C₁₆H₁₉N 225, observed m/e 226 (M+H)⁺ (2.0 min).

Diastereomer β: LC-MS: calculated for C₁₆H₁₉N 225, observed m/e 226 (M+H)⁺ (1.9 min).

### Reference Example 3

### 3-Amino-1,2-diphenylpentane hydrochloride salt

Diastereomer α: LC-MS: calculated for C₁₇H₂₁N 239, observed m/e 240 (M+H)⁺ (2.1 min).

Diastereomer β: LC-MS: calculated for C₁₇H₂₁N 239, observed m/e 240 (M+H)⁺ (2.0 min).

### Reference Example 4

### 1-Amino-1,2,3-triphenylpropane p-toluenesulfonate salt

Diastereomer α: LC-MS: calculated for C₂₁H₂₁N 287, observed m/e 288 (M+H)⁺ (2.3 min).

Diastereomer β: LC-MS: calculated for C₂₁H₂₁N 287, observed m/e 288 (M+H)⁺ (2.3 min).

### Reference Example 5

### 2-Amino-4-(4-chlorophenyl)-3-phenylbutane hydrochloride salt

Diastereomer α: LC-MS: calculated for C₁₆H₁₈ClN 259, observed m/e 260 (M+H)⁺ (2.3 min).

Diastereomer β: LC-MS: calculated for C₁₆H₁₈ClN 259, observed m/e 260 (M+H)⁺ (2.2 min).

### Reference Example 6

### 2-Amino-3-(4-chlorophenyl)-4-phenylbutane hydrochloride salt

Diastereomer α: LC-MS: calculated for C₁₆H₁₈ClN 259, observed m/e 260 (M+H)⁺ (2.3 mm).

Diastereomer β: LC-MS: calculated for C₁₆H₁₈ClN 259, observed m/e 260 (M+H)⁺ (2.1 min).

### Reference Example 7

### 2-Amino-4-(4-methoxycarbonylphenyl)-3-phenylbutane hydrochloride salt

Diastereomer α: LC-MS: calculated for C₁₈H₂₁NO₂ 283, observed m/e 284 (M+H)⁺ (2.0 min).

Diastereomer β: LC-MS: calculated for C₁₈H₂₁NO₂ 283, observed m/e 284 (M+H)⁺ (1.9 min).

### Reference Example 8

### 2-Amino-3-(2-Chlorophenyl)-4-phenylbutane (mixture of diastereomers α/β 1:2)

LC-MS: calculated for C₁₆H₁₈ClN 259, observed m/e 260 (M+H)⁺ (1.9/2.0 min).

### Reference Example 9

### 2-Amino-3-(4-methoxyphenyl)-4-phenylbutane (mixture of diastereomers α/β 2:5)

LC-MS: m/e 256 (M+H)⁺ (1.7 min).

### Reference Example 10

### N-[3-(4-Chlorophenyl)-2-phenyl-1-methylpropyl]-amine hydrochloride (Diastereomer α)

This compound was prepared according to the procedures in WO 05/044785, Reference Example 10. ¹H NMR (500 MHz, CD₃OD): δ 7.35-6.98 (m, 9H), 3.62 (m, 1H), 3.20 (dd, 1H), 3.05 (m, 1H), 2.98 (dd, 1H), 1.19 (d, 3H). LC-MS: m/e 260 (M+H)⁺ (2.3 min).

### Reference Example 11

### N-[3-(4-Chlorophenyl)-2(S)-phenyl-1(S)-methylpropyl]-amine hydrochloride

This compound was prepared according to the procedures in WO 05/044785, Reference Example 11. ¹H NMR (500 MHz, CD₃OD): δ 7.35-6.98 (m, 9H), 3.62 (m, 1H), 3.20 (dd, 1H), 3.05 (m, 1H), 2.98 (dd, 1H), 1.19 (d, 3H). LC-MS: m/e 260 (M+H)⁺ (2.3 min).

### Reference Example 12

### 2-Amino-4-(4-chlorophenyl)-3-(3-fluorophenyl)butane hydrochloride salt (Mixture of Diastereomers of α/β 5:1)

This compound was prepared according to the procedures in WO 05/044785, Reference Example 12. ¹H NMR (400 MHz, CD₃OD): δ 7.32-6.90 (m, 7H), 3.61 (m, 1H), 3.20 (dd, 1H), 3.11 (m, 1H), 2.92 (dd, 1H), 1.19 (d, 3H). LC-MS: m/e 278 (M+H)⁺ (2.4 min).

The amines of Reference Examples 13-16 were prepared according to the procedures described in Reference Example 12, and as described in WO 05/044785.

### Reference Example 13

### 2-Amino-4-(4-chlorophenyl)-3-(2-fluorophenyl)butane hydrochloride salt (mixture of diastereomers α/β 10:1)

LC-MS: m/e 278 (M+H)⁺ (2.3 min).

### Reference Example 14

### 2-Amino-4-(4-chlorophenyl)-3-(4-fluorophenyl)butane hydrochloride salt (Mixture of Diastereomers α/β 10:1)

LC-MS: m/e 278 (M+H)⁺ (2.5 min).

### Reference Example 15

### 2-Amino-4-(4-chlorophenyl)-3-(2-pyridyl)butane hydrochloride salt (Mixture of Diastereomers α/β 10:1)

LC-MS: m/e 261 (M+H)⁺ (1.6 min).

### Reference Example 16

### 2-Amino-4-(4-chlorophenyl)-3-(4-pyridyl)butane hydrochloride salt (Mixture of Diastereomers α/β 10:1)

Trimethyl aluminum was used in place of dimethylaluminum chloride at Step B of Reference Example 12 LC-MS: m/e 261 (M+H)⁺.

### Reference Example 17

### 2-Amino-4-(4-cyanophenyl)-3-phenylbutane hydrochloride salt (Mixture of Diastereomers α/β 10:1)

This compound was prepared according to the procedures in WO 05/044785, Reference Example 17. LC-MS: m/e 251 (M+H)⁺ (1.9 min).

### Reference Example 18

### 2-Amino-4-(5-chloro-2-pyridyl)-3-phenylbutane hydrochloride salt (Mixture of Diastereomers α/β 10:1)

This compound was prepared according to the procedures in WO 05/044785, Reference Example 18. LC-MS: m/e 261 (M+H)⁺.

### Reference Example 19

### N-[3-(4-chlorophenyl)-2-(3-pyridyl)-1-methylpropyl]-amine, hydrochloride (Mixture of Diastereomers α/β 10:1)

This compound was prepared according to the procedures in WO 05/044785, Reference Example 19. LC-MS: m/e 261 (M+H)⁺ (1.2 min).

### Reference Example 20

### 2-Amino-4-(2,4-dichlorophenyl)-3-(4-chlorophenyl)butane hydrochloride salt (3 isomers)

This compound was prepared according to the procedures in WO 05/044785, Reference Example 20. Isomer 1: ¹H NMR (500 MHz, CD₃OD): δ 7.35 (d, 1H), 7.29 (d, 2H), 7.15 (d, 2H), 7.06 (dd, 1H), 6.91 (d, 1H), 3.68 (m, 1H), 3.36 (dd, 1H), 3.06 (dd, 1H), 1.18 (d, 3H). LC-MS: m/e 328 (M+H)⁺ (2.8 min).

The two slower co-eluting isomers were treated in the same fashion to give two other isomers of the title compound. Isomer 2 and 3 (1:1): LC-MS: m/e 328 (M+H)⁺ (2.7/2.8 min).

### Reference Example 21

### 2-Amino-4-(4-chloro-2-fluorophenyl)-3-(4-chlorophenyl)butane hydrochloride salt (Isomers, 1, 2 and 3)

These compounds were prepared according to the procedures in WO 05/044785, Reference Example 21. Isomer 1: LC-MS: m/e 312 (M+H)⁺ (2.6 min). Isomer 2 and 3 (1:1): LC-MS: m/e 312 (M+H)⁺ (2.5/2.6 min).

### Reference Example 22

### 2-(4-Chlorophenyloxy)-2-(4-chlorophenyl)ethylamine hydrochloride salt

This compound was prepared according to the procedures in WO 05/044785, Reference Example 22.

¹H NMR (500 MHz, CD₃OD): δ 7.46-7.40 (m, 4H), 7.20 (d, 2H), 6.91 (d, 2H), 5.53 (m, 2H), 3.36 (m, 2H). LC-MS: m/e 282 (M+H)⁺ (2.5 min).

### Reference Example 23

### 2,2-Bis(4-chlorophenyl)ethylamine hydrochloride salt

This compound was prepared according to the procedures in WO 05/044785, Reference Example 23. ¹H NMR (500 MHz, CD₃OD): δ 7.40-7.34 (m, 4H), 4.28 (m, 1H), 3.62 (d, 2H). LC-MS: m/e 266 (M+H)⁺ (2.3 min).

### Reference Example 24

### 2-Amino-3-(4-chlorophenylthio)-3-(4-chlorophenyl)propane hydrochloride salt (Two Diastereomers)

This compound was prepared according to the procedures in WO 05/044785, Reference Example 24. LC-MS: m/e 312 (M+H)⁺ (2.7 min).

### Reference Example 25

### 2-Amino-3,4-bis(4-chlorophenyl)-2-methylbutane hydrochloride salt

This compound was prepared according to the procedures in WO 05/044785, Reference Example 25.

¹H NMR (500 MHz, CD₃OD): (free amine) δ 7.22-7.14 (m, 4H), 7.06 (d, 2H), 6.96 (d, 2H), 3.22 (dd, 1H), 2.95 (dd, 1H), 2.86 (dd, 1H), 1.16 (s, 3H), 1.10 (s, 3H).

### Reference Example 26

### 2-Amino-5-methyl-3-phenylhexane hydrochloride salt

This compound was prepared according to the procedures in WO 05/044785, Reference Example 39

¹H NMR: (500 MHz, CDCl₃): δ 0.86 (m, 6H), 0.99 (d, 3H), 1.25 (m, 1H), 1.54 (m, 1H), 1.77 (m, 1H), 2.73 (m, 1H), 3.19 (m, 1H), 7.2-7.4 (m, 5H).

### Reference Example 27

### N-[3-(4-Chlorophenyl)-2-(3,5-difluorophenyl)-1-methylpropyl]-amine hydrochloride (Diastereomer α)

This compound was prepared according to the procedures in WO 05/044785, Reference Example 27. LC-MS: m/e 296 (M+H)⁺ (2.39 min).

### Reference Example 28

### N-[2-(3-Bromophenyl)-3-(4-chlorophenyl)-1-methylpropyl]amine hydrochloride (Diastereomer α)

This compound was prepared according to the procedures in WO 05/044785, Reference Example 28. LC-MS: m/e 338 (M+H)⁺ (2.5 min).

### Reference Example 29

### N-[3-(4-Chlorophenyl)-2-(3-cyanophenyl)-1-methylpropyl]amine hydrochloride (Diastereomer α)

### Step A 2-(N-tert-Butoxycarbonyl)amino-4-(4-chlorophenyl)-3-(3-cyanophenyl)butane

To a solution of 2-(N-tert-butoxycarbonyl)amino-3-bromophenyl-4-(4-chlorophenyl)butane (Intermediate of Reference Example 471.0 g, 2.3 mmol) in 5 mL DMF was added zinc cyanide (0.16 g, 1.4 mmol), tris(dibenzylidene-acetone)dipalladium chloroform complex (3.0 mg, 2.8 μmol), 1,1′-bis(diphenyl p-hosphino)ferrocene (5.0 mg, 9.0 μmol) and water (0.1 mL). After heating at 120° C. for 6 h under nitrogen, another batch of zinc cyanide (0.16 g, 1.4 mmol), tris(dibenzylideneacetone)dipalladium chloroform complex (5.0 mg, 4.8 μmol), 1,1′-bis(diphenylphosphino)ferrocene (5.0 mg, 9.0 μmol) and water (0.05 mL) was added, and heating was continued for another 18 h. After cooling to room temperature, the resulting mixture was partitioned between water (50 mL) and ether (50 mL). The organic layer was separated and the aqueous layer extracted with ether (2×50 mL). The combined extracts were dried over anhydrous MgSO₄, filtered and concentrated, and the residue was purified by flash column chromatography on silica gel eluted with 20% EtOAc in hexane to afford the title compound. ¹H NMR (400 MHz, CD₃OD): δ 7.6-7.3 (m, 4H), 7.10 (d, 2H), 6.92 (d, 2H), 3.88 (m, 1H), 3.20 (m, 1H), 2.97 (m, 1H), 1.82 (m, 1H), 1.45 (s, 9H), 0.94 (d, 3H). LC-MS: m/e 385 (M+H)⁺ (3.9 min).

### Step B N-[3-(4-Chlorophenyl)-2-(3-cyanophenyl)-1-methylpropyl]amine hydrochloride (Diastereomer α)

The title compound was prepared following the procedure described for Reference Example 10, Step I. LC-MS: m/e 285 (M+H)⁺ (2.2 min).

### Reference Example 30

### N-[2-(3-Chlorophenyl)-3-(4-chlorophenol)-1-methylpropyl]amine hydrochloride (Diastereomer α)

This compound was prepared according to the procedures in WO 05/044785, Reference Example 29.

LC-MS: m/e 294 (M+H)⁺ (2.82 min).

### Reference Example 31

### N-[2-(3-Bromophenyl)-3-(4-chlorophenyl)-1-methylpropyl]amine hydrochloride and N-[3-(4-Chlorophenyl)-2-(3-iodophenyl)-1-methylpropyl]amine hydrochloride (1:1 Mixture) (Diastereomer α)

This compound was prepared according to the procedures in WO 05/044785, Reference Example 30. LC-MS: m/e 338/386/(M+H)⁺ (2.6 min).

### Reference Example 32

This compound was prepared according to the procedures in WO 05/044785, Reference Example 31.

TLC R_{f}=0.12 (5% MeOH in CH₂Cl₂). 500 MHz ¹H NMR (CDCl₃): δ 1.16 (t, 3H); 1.67 (m, 2H); 1.85 (m, 3H); 2.01 (m, 2H); 2.48 (m, 1H); 2.74 (m, 2H); 2.90 (dd, 1H); 3.15 (d quint, 2H); 3.37 (m, 2H).

2-Amino-4-(4-chlorophenyl)-3-methoxy-butane, 2-amino-4-(4-chlorophenyl)-3-ethoxy-butane, 2-amino-4-(4-chlorophenyl)-3-n-propyloxy-butane, 2-amino-4-(4-chlorophenyl)-3-n-pentyloxy-butane, and 2-amino-4-(4-chlorophenyl)-3-cyclopentylmethoxy-butane were prepared according to the procedures described in 05/044785, Reference Example 31, substituting an appropriate alcohol for cyclobutylmethanol in Step B.

### Reference Example 33

### 2-Amino-4-(4-chlorophenyl)-3-(1-pyrrolidinyl)-butane hydrochloride

This compound was prepared according to the procedures in WO 05/044785, Reference Example 32.

LC/MS m/e=253 (M+1). 500 MHz ¹H NMR (CD₃OD) δ 1.56, 1.59 (2d, J=7.2 Hz, 311), 2.03 (m, 6H), 2.08 (m, 2H), 3.20-4.00 (m, 3H), 7.43 (m, 4H).

### Reference Example 34

Benzyl-3-amino-2-(4-chlorobenzyl)butyrate.

This compound was prepared according to the procedures in WO 05/044785, Reference Example 33. Proton NMR spectrum show that the two pairs of diastereomers are obtained in ˜1:1 ratio, homogeneous by TLC, R_{f}=0.4 in 95:5 CH₂Cl₂: MeOH. LC/MS m/e=318 (M+1). 400 MHz ¹H NMR (CDCl₃) δ 1.27, 1.29 (2d, J=7 Hz, 3H), 2.85 (m, 1H), 3.03 (m, 1H), 3.15 (m, 1H), 3.55 (m, 1H), 4.85 (br, 2H), 5.00-5.18 (m, 2H), 7.0-7.2 (m, 9H).

### Reference Example 35

### 2-Amino-4-(4-chlorophenyl)-3-cyclopentylbutane

This compound was prepared according to the procedures in WO 05/044785, Reference Example 34.

LC/MS m/e 251.9 (M+1); 500 MHz ¹H NMR (CDCl₃): δ 0.93 (m, 1H), 1.29 (q, 3H), 1.29 (m, 2H), 1.61 (m, 4H), 1.87 (m, 3H), 2.62 (m, 1H), 2.80 (m, 1H), 3.26 and 3.48 (m, 1H).

2-Amino-4-(4-chlorophenyl)-3-ethyl-butane and 2-amino-4-(4-chlorophenyl)-3-isopropyl-butane were also prepared according to the procedures described in Reference Example 35 substituting the appropriate ester for methyl cyclopentylacetate in Step A.

### Reference Example 36

### 2-Amino-3-(1-(1,2,3-triazolyl))-4-(4-chlorophenyl)butane

This compound was prepared according to the procedures in WO 05/044785, Reference Example 35. ¹H NMR (400 MHz, CDCl₃): δ 1.085-1.174 (d's 311), 3.220-3.361 (m, 2H), 3.517-3.563 (m, 1H), 4.379-4.431 (m, 1H), 6.679-7.179 (d's, 4H), 7.297, 7.40, 7.592 & 7.607 (s's, 2H).

### Reference Example 37

### 2-Amino-3-(1-(1,2,4-triazolyl)-4-(4-chlorophenyl)butane

This compound was prepared according to the procedures in WO 05/044785, Reference Example 36.

### Reference Example 38

### N-[3-(4-Chlorophenyl)-2-(3-methylphenyl)-1-methylpropyl]amine hydrochloride (Diastereomer α)

### Step A 2-(N-tert-Butoxycarbonyl)amino-4-(4-chlorophenyl)-3-(3-methylphenyl)butane

This compound was prepared according to the procedures in WO 05/044785, Reference Example 37. LC-MS: m/e 274 (M+H)⁺ (2.5 min).

### Reference Example 39

### N-[3-(4-Chlorophenyl)-2-(3-trifluoromethylphenyl)-1-methylpropyl]amine hydrochloride (Diastereomer α)

This compound was prepared according to the procedures in WO 05/044785, Reference Example 38. LC-MS: m/e 328 (M+H)⁺ (2.6 min).

### Reference Example 40

### N-[37(5-Chloro-2-pyridyl)-2(S)-phenyl-1(S)-methylpropyl]amine hydrochloride (Diastereomer α)

This compound was prepared according to the procedures in WO 05/044785, Reference Example 39. ¹H NMR (500 MHz, CD₃OD): δ 8.75 (d, 1H), 8.19 (dd, 1H), 7.55 (d, 1H), 7.4-7.2 (m, 5H), 3.78 (m, 1H), 3.62 (dd, 1H), 3.48 (m, 1H), 3.43 (dd, 1H), 1.22 (d, 3H). LC-MS: m/e 261 (M+H)⁺ (2.2 min).

### Reference Example 41

### N-[2-(3-Bromophenyl)-3-(5-chloro-2-pyridyl)-1-methylpropyl]amine hydrochloride (Diastereomer α)

This compound was prepared according to the procedures in WO 05/044785, Reference Example 40. LC-MS: m/e 338 (M+H)⁺ (2.3 min).

### Reference Example 42

### N-[3-(5-Chloro-2-pyridyl)-2-(3-chlorophenyl)-1-methylpropyl]amine hydrochloride (Diastereomer α)

This compound was prepared according to the procedures in WO 05/044785, Reference example 41. LC-MS: m/e 295 (M+H)⁺ (2.0 min).

### Reference Example 43

### N-[2-(5-Bromo-2-pyridyl)-3-(4-chlorophenyl)-1-methylpropyl]amine hydrochloride (Diastereomer α)

This compound was prepared according to the procedures in WO 05/044785, Reference Example 42. LC-MS: m/e 339 (M+H)⁺ (2.5 min).

### Reference Example 44

### N-[2-(5-Bromo-3-pyridyl)-3-(4-fluorophenyl)-1-methylpropyl]amine hydrochloride (Diastereomer α)

This compound was prepared according to the procedures in WO 05/044785, Reference example 43. LC-MS: m/e 323 (M+H)⁺ (2.3 min).

### Reference Example 45

### N-[3-(4-Chlorophenyl)-2-(5-cyano-3-pyridyl)-1-methylpropyl]amine hydrochloride (Diastereomer α)

### Step A 5-Cyano-3-pyridylacetone

This compound was prepared according to the procedures in WO 05/044785, Reference example 44. LC-MS: m/e 286 (M+H)⁺ (1.9 min).

### Reference Example 46 N-[2-(5-Cyano-3-pyridyl)-3-(4-fluorophenyl)-1-methylpropyl]amine hydrochloride (Diastereomer α)

This compound was prepared according to the procedures in WO 05/044785, Reference example 45. LC-MS: m/e 270 (M+H)⁺ (2.2 min).

### Reference Example 47

### N-[2-(5-Cyano-3-pyridyl)-3-(3,4-difluorophenyl)-1-methylpropyl]amine hydrochloride (Diastereomer α)

This compound was prepared according to the procedures in WO 05/044785, Reference example 46. LC-MS: m/e 288 (M+H)⁺ (2.3 min).

### Reference Example 48

### N-[3-(3-Chlorophenyl)-2-(5-cyano-3-pyridyl)-1-methylpropyl]amine hydrochloride (Diastereomer α)

This compound was prepared according to the procedures in WO 05/044785, Reference example 47. LC-MS: m/e 286 (M+H)⁺ (2.4 min).

### Reference Example 49

### N-[3-(4-Chlorophenyl)-2-(5-chloro-3-pyridyl)-1-methylpropyl]amine hydrochloride (Diastereomer α)

This compound was prepared according to the procedures in WO 05/044785, Reference example 48. LC-MS: m/e 295 (M+H)⁺ (1.9 min).

### Reference Example 50

### N-[2-(5-Chloro-3-pyridyl)-3-(4-fluorophenyl)-1-methylpropyl]amine hydrochloride (Diastereomer α)

This compound was prepared according to the procedures in WO 05/044785, Reference Example 49. LC-MS: m/e 279 (M+H)⁺ (2.3 min).

### Reference Example 51

### 2-Amino-3-(5-chloro-3-pyridyl)-5-methylhane, Hydrochloride Salt (Diastereomer α/β 6:1)

This compound was prepared according to the procedures in WO 05/044785, Reference example 50. LC-MS: m/e 227 (M+H)⁺ (2.2 min).

### Reference Example 52

### N-[2-(5-Chloro-3-pyridyl)-3-cyclobutyl-1-methylpropyl]amine hydrochloride (Diastereomer α/β 6:1)

This compound was prepared according to the procedures in WO 05/044785, Reference example 51. LC-MS: m/e 239 (M+H)⁴. (2.3 min).

### Reference Example 53

### N-[3-(4-Chlorophenyl)-2-(3-cyanophenyl)-1-methylpropyl]amine hydrochloride (Diastereomer α)

### Step A 3-Cyanophenylacetone

The title compound was prepared following the procedure described for Reference Example 43 substituting 3,5-dibromopyridine with 3-bromobenzonitrile and 2-(diphenylphosphino)-2′-(N,N-dimethylamino)biphenyl with 2-(dicyclohexylphosphino)-2′-(N,N-dimethylamino)biphenyl at Step A. ¹H NMR (500 MHz, CD₃OD): δ 7.6 (m, 1H), 7.56 (br s, 1H), 7.50-7.48 (m, 2H), 3.88 (s, 2H), 2.21 (s, 3H).

### Step B N-[3-(4-Chlorophenyl)-2-(3-cyanophenyl)-1-methylpropyl]amine hydrochloride (Diastereomer α)

The title compound was prepared following the procedure described for Reference Example 43 substituting 5-bromo-3-pyridylacetone with 3-canophenylacetone at Step B. LC-MS: m/e 285 (M+H)⁺ (2.2 min).

### Reference Example 54

### N-[3-(4-Chlorophenyl)-2-(5-fluoro-3-pyridyl)-1-methylpropyl]amine hydrochloride (Diastereomer α)

This compound was prepared according to the procedures in WO 05/044785, Reference example

### Example 53

LC-MS: m/e 279 (M+H)⁺ (2.4 min).

### Reference Example 55

### N-[3-(4-Chlorophenyl)-2-(5-methyl-3-pyridyl)-1-methylpropyl]amine hydrochloride (Diastereomer α)

This compound was prepared according to the procedures in WO 05/044785, Reference Example 54. LC-MS: m/e 275 (M+H)⁺ (1.3 min).

### Reference Example 56

### N-[2-(3-Bromo-5-fluorophenyl)-3-(4-Chlorophenyl)-1-methylpropyl]amine hydrochloride (Diastereomer α)

This compound was prepared according to the procedures in WO 05/044785, Reference Example 55. LC-MS: m/e 356 (M+H)⁺ (2.9 min).

### Reference Example 57

### N-[2-(3-Bromo-5-fluorophenyl)-3-(4-fluorophenyl)-1-methylpropyl]amine hydrochloride (Diastereomer α)

This compound was prepared according to the procedures in WO 05/044785, Reference example 56. LC-MS: m/e 340 (M+H)⁴ (2.8 min).

### Reference Example 58

### 2-Amino-3-indolin-N-yl-4(4-chloro)phenylbutane

This compound was prepared according to the procedures in WO 05/044785, Reference example 57. LC/MS m/e=302 (M+1). ¹H NMR (500 MHz, CDCl₃): δ 1.13, 1.14 (2d, J=6.5 Hz, 1H), 1.55-1.60 (m, 2H), 2.80-3.10 (m, 4H), 3.30-3.60 (m, 2H), 6.348 and 6.38 (2d, J=7.9 Hz, 1H), 6.50-6.78 (m, 2H), 6.95-7.24 (m, 5H)

### Reference Example 59

### 2-Amino-3-indol-N-yl-4(4-chloro)phenylbutane

This compound was prepared according to the procedures in WO 05/044785, Reference Example 58. LC/MS: calculated for C₁₈H₁₉ClN₂ 299, observed m/e 300 (M+H)⁺ (2.4 min).

### Reference Example 60

### 2-Amino-3-(N-methyl, N-phenyl)amino-4(4-chloro)phenylbutane

This compound was prepared according to the procedures in WO 05/044785, Reference Example 59. LC/MS: calculated for C₁₇H₂₁ClN₂ 289, observed m/e 290 (M+H)⁺ (2.4 min).

### Reference Example 61

### 2-Amino-3-(7-azaindol-N-yl)-4(4-chloro)phenylbutane

This compound was prepared according to the procedures in WO 05/044785, Reference Example 60. LC/MS: calculated for C₁₇H₁₈ClN₃ 300, observed m/e 301 (M+H)⁺ (2.7 min).

### Reference Example 62

### 2-Amino-3-(benzisoxazol-3-yl)-4(4-chloro)phenylbutane

This compound was prepared according to the procedures in WO 05/044785, Reference Example 61. LC/MS: calculated for C₁₇H₁₇ClN₂₀ 300, observed m/e 301 (M+H)⁺ (2.2 min).

### Reference Example 63

### 4-(4-Methylphenyl)-3-phenylbutan-2-amine (Mixture of 4 Isomers)

This compound was prepared according to the procedures in WO 05/044785, Reference Example 62. LC-MS: m/e 240 (M+H)⁺ (2.22 min).

### Reference Example 64

### 4-(4-Methoxyphenyl)-3-phenylbutan-2-amine

This compound was prepared according to the procedures in WO 05/044785, Reference Example 63. LC-MS: m/e 256 (M+H)⁺ (1.90 and 2.03 min).

### Reference Example 65

### 3-[2-Amino-1-(4-fluorobenzyl)propyl]benzonitrile

Prepared using the procedures described in Reference Example 53 using 3-(2-oxopropyl)benzonitrile and 1-(chloromethyl)-4-fluorobenzene as the reactants in Step B. LC-MS: m/e 269 (M+H)⁺ (2.87 min).

### Reference Example 66

### N-[2-Phenyl-3-(4-fluorophenyl)-1-methylpropyl]amine hydrochloride (Diastereomer α)

This compound was prepared according to the procedures in WO 05/044785, Reference example 65. LC-MS, Rₜ=2.2 min, m/e=244.

### Reference Example 67

### 2-(2,3-Dihydro-1-H-indol-1-yl)-1,4-dimethylpentylamine

This compound was prepared according to the procedures in WO 05/044785, Reference example 66. LC-MS, Rₜ=2.24 min, m/e=233.

The following amines were synthesized by the method of Reference Example 67.

### Reference Example 68

### 3-Cyclobutyl-2-(3,4-dihydroquinoline-1(2H)-yl)-1-methylpropylamine

LC-MS, Rₜ=2.8 min, m/e=259.

### Reference Example 69

### 2-(3,4-Dihydroquinoline-1(2H)-yl)-1,4-dimethylpentylamine

LC-MS, Rₜ=2.74 min, m/e=248.

### Reference Example 70

### 2-(1H-1,2,3-Benzotriazol-1-yl)-3-(4-chlorophenyl)-1-methylpropylamine

This compound was prepared according to the procedures in WO 05/044785, Reference Example 69. LC-MS, Rₜ=2.0 min, m/e=301.

### Reference Example 71

### 3-(4-Chlorophenyl)-2-(thien-3-yl)-1-methylpropylamine

This compound was prepared according to the procedures in WO 05/044785, Reference example 70. LC-MS, Rₜ=2.19 min, m/e=266.

### Reference Example 72

### 3-(4-Chlorophenyl)-2-(thien-2-yl)-1-methylpropylamine

This compound was prepared according to the procedures in WO 05/044785, Reference Example 71. LC-MS, Rₜ=2.18 min, m/e=266.

### Reference Example 73

### 3-(4-Chlorophenyl)-1-methyl-2-(1-methyl-1H-indol-3-yl)propylamine

The title compound was prepared according to the method described in Reference Example 72. LC-MS: Rₜ=2.5 min, m/e=313.

### Reference Example 74

### 3-(4-Chlorophenyl)-1-methyl-2-(1H-indazol-1-yl)propylamine

This compound was prepared according to the procedures in WO 05/044785, Reference Example 73. LC-MS: Rₜ=2.24 min, m/e=300.

### Reference Example 75

### 3-(4-Chlorophenyl)-1-methyl-2-(1-methyl-1H-indol-4-yl)propylamine

This compound was prepared according to the procedures in WO 05/044785, Reference Example 74. LC-MS, Rₜ=2.4 min, m/e=313.

### Reference Example 76

### 3-(4-Chlorophenol)-1-methyl-2-(pyridazin-3-yl)propylamine

This compound was prepared according to the procedures in WO 05/044785, Reference example 75. LC-MS, Rₜ=1.63 min, m/e=262.

### Reference Example 77

### 3-(4-Chlorophenyl)-1-methyl-2-(pyrimidin-5-yl)propylamine

This compound was prepared according to the procedures in WO 05/044785, Reference example 76, LC-MS, Rₜ=1.57 min, m/e=262.

### Reference Example 78

### 2-(3-Cyanophenyl)-3-cyclobutyl-1-methylpropylamine

This compound was prepared according to the procedures in WO 05/044785, Reference example 77. LC-MS, Rₜ=2.48 min, m/e=229.

The compounds of Reference Examples 79-81 were obtained by procedures described in Reference Example 78.

### Reference Example 79

### 2-(3-Cyanophenyl)-3-cyclopropyl-1-methylpropylamine

LC-MS, Rₜ=1.8 min, m/e=215.

### Reference Example 80

### 2-(3-Cyanophenyl)-3-cyclopentyl-1-methylpropylamine

LC-MS, Rₜ=2.7 min, m/e=243.

### Reference Example 81

### 2-(3-Cyanophenyl)-3-cyclohexyl-1-methylpropylamine

LC-MS, Rₜ=2.8 min, m/e=257.

### Reference Example 82

### 2-(3-Cyanophenyl)-3-(1-tert-butyloxycarbonyl-piperidin-4-yl)-1-methylpropylamine

### Step A 3-(3-Cyanophenyl)-4-(1-tert-butyloxycarbonyl-piperidin-4-yl)-butan-2-one

The title compound was synthesized by the method of Reference Example 78, Steps A-B.

### Step B 2-(3-Cyanophenyl)-3-(1-tert-butyloxycarbonyl-piperidin-4-yl)-1-methylpropylamine

The title amine was obtained by the method of Reference Example 10, steps E-G except that di-tert-butyl dicarbonate was not added in Step G. LC-MS, Rₜ=2.72 min, m/e=258 (M-99).

### Reference Example 83

### N-[3-(4-Chlorophenyl)-2-(3-methylthiophenyl)-1-methylpropyl]amine hydrochloride (Diastereomer α)

The title compound was prepared following the same procedure as described in Example 43 substituting 3,5-dibromopyridine with 3-bromothioanisole at Step A. LC-MS: m/e 306 (M+H)⁺ (2.68 min).

### Reference Example 84

### N-[3-(4-Chlorophenyl)-2(S)-(3-cyanophenyl)-1(S)-methylpropyl]amine hydrochloride

### Step A 3-(4-Chlorophenyl)-2-(3-bromophenyl)propanoic acid, (S)-methylbenzylamine Salt

To a solution of 3-bromophenylacetic acid (3.3 kg, 15 mol) and p-chlorobenzyl chloride (2.6 kg, 16 mol) in 6.5 L of THF at −28° C. was added lithium bis(trimethylsilyl)amide (1 M in tetrahydrofuran, 31 L, 31 mol). The reaction mixture was aged at 0° C. for 2 h, and was then quenched with 2.5 N HCl (18 L). The organic layer was washed with water (8 L), concentrated on a rotary evaporator, and the residue was diluted with 10 L of toluene. One equivalent of (S)-methylbenzylamine was then added, and the precipitate was collected by filtration and dried under a nitrogen current for 12 h to give the salt of the racemic acid. Recrystallization from methanol provided the title compound enriched in a single enantiomer of the acid.

### Step B 4-(4-Chlorophenyl)-3-(3-bromophenyl)-2-butanone

To 3-(4-chlorophenyl)-2-(3-bromophenyl)propanoic acid, (S)-methylbenzylamine salt (2.2 kg, 4.7 mol) in toluene (39 L) and water (38 L) was added 5 N HCl (1.5 L). After stirring for 30 min, the organic layer was separated and concentrated to 20 L, and was added N,N-dimethylformamide (16 ml) and oxalyl chloride (0.49 L, 5.6 mol) over 1 h. After stirring for 30 min, the resulting crude acyl chloride was slowly transferred into a mixture of water (13 L), potassium carbonate (2.6 kg, 19 mol) and N-methoxy-N-methylamine hydrochloride (0.69 kg, 7.1 mol). After stirring for 30 min, the reaction was diluted with water (12 L), and the organic layer was separated, dried over sodium sulfate and concentrated to an oil, which was used without further purification. To the oil thus obtained in toluene (16 L) and tetrahydrofuran (5 L) at 10° C. was added methylmagnesium chloride (3 M in tetrahydrofuran, 2.0 L, 6.0 mol) over 1 h. After stirring for 30 min, the reaction was quenched by addition of 47 L of 20% aqueous ammonium chloride until the pH reached 7.5. The organic layer was separated, washed with water (16 L), dried over sodium sulfate and concentrated to give the title compound as an oil. ¹H NMR (500 MHz, CD₃OD): δ 7.40 (d, 1H), 7.36 (s, 1H), 7.28-7.10 (m, 4H), 7.04 (d, 2H), 4.06 (dd, 1H), 3.28 (dd, 1H), 2.84 (dd, 1H), 2.03 (s, 3H).

### Step C 4-(4-Chlorophenyl)-3-(3-bromophenyl)-2-butanol

To a solution of 4-(4-chlorophenyl)-3-(3-bromophenyl)-2-butanone (1.55 kg, 4.6 mol) in tetrahydrofuran (8.4 L) at −60° C. was added L-selectride (1.0 M in tetrahydrofuran, 5.3 L, 5.3 mol), and the reaction mixture was allowed to slowly warm up to room temperature overnight. The mixture was cooled back to 0° C., and was quenched by slow addition of acetone, aqueous sodium hydroxide (2.5 N, 8.5 L, 21 mol) and 30% hydrogen peroxide (2.1 L, 21 mol). After stirring at room temperature for 13 h, the reaction mixture was diluted with toluene (30 L). The organic layer was separated, washed with water (2×12 L), and concentrated to give the title compound as an oil. ¹H NMR (500 MHz, CD₃OD): δ 7.40 (s, 1H), 7.30 (d, 1H), 7.28-7.10 (m, 4H), 7.04 (d, 2H), 3.98 (m, 1H), 3.12 (dd, 1H), 2.90 (dd, 1H), 2.80 (m, 1H), 1.08 (d, 3H).

### Step D 4-(4-Chlorophenyl)-3-(3-cyanophenyl)-2-butanol

To a solution of 4-(4-chlorophenyl)-3-(3-bromophenyl)-2-butanol (1.5 kg, 4.6 mol) in dimethylformamide/water (volume ratio 99:1, 15 L total) was added zinc cyanide (0.39 kg, 3.3 mol), 1,1′-bis(diphenylphosphino)ferrocene (115 g, 0.21 mol) and tris(dibenzylideneacetone)dipalladium (76 g, 0.08 mol). The reaction mixture was stirred and degassed at room temperature for 1 h, and heated at 115° C. for 6 h before cooling to room temperature. Tributylphosphine (46 mL, 0.17 mol) was then added. After stirring for 1 h, the reaction was quenched with aqueous ammonia (1.56 L). After stirring for 1 h, the mixture was filtered through solka floc, and the cake was washed with isopropyl acetate. The filtrate was washed with water (5 L), and the aqueous layer was extracted with isopropyl acetate (4 L). The organic layers were combined, washed with water (10 L×2) and concentrated to give the title compound, which was used for the ensuing reaction after azeotroping with toluene.

### Step E 4-(4-Chlorophenyl)-3-(3-cyanophenyl)-2 methylsulfonyloxybutane

To a solution of 4-(4-chlorophenyl)-3-(3-cyanophenyl)-2-butanol (1.0 kg, 3.5 mol) in toluene (10 L) at 0° C. was added triethylamine (684 mL, 4.9 mol) and methanesulfonyl chloride (353 mL, 4.6 mol). After stirring for 5 min, the reaction mixture was filtered, and the precipitate was washed with toluene (8 L). The filtrate was washed with sodium bicarbonate (6 L, 50% saturated aqueous solution) and water (3 L), and concentrated to give the title compound.

### Step F 4-(4-Chlorophenyl)-3-(3-cyanophenyl)-2-azidobutane

To a solution of 4-(4-chlorophenyl)-3-(3-cyanophenyl)-2 methylsulfonyloxybutane (1.1 kg, 2.9 mol) in N,N-dimethylformamide (4.1 L) was added sodium azide (378 g, 5.8 mol), and the reaction was heated at 70° C. for 7 h. After cooling to room temperature, the reaction mixture was diluted with isopropyl acetate (11 L) and was washed with sodium bicarbonate (50% saturated aqueous solution) and water (5.5 L). The organic layer was separated and treated with Darco KB (254 g) overnight. The mixture was filtered over solka floc, washed with toluene and concentrated. The residue was diluted with toluene (1 L) and was loaded onto a silica gel pad, which was eluted with 90:10 hexane/ethyl acetate to give the title compound.

### Step G N-[3-(4-Chlorophenyl)-2(S)-(3-cyanophenyl)-1(S)-methylpropyl]amine

To a solution of 4-(4-chlorophenyl)-3-(3-cyanophenyl)-2-azidobutane (650 g, 2.1 mol) in isopropyl acetate (3.3 L) was added Lindlar's catalyst (130 g), and the mixture was hydrogenated at 40 psi at 45° C. for 24 h and at room temperature for anther 48 h. The reaction mixture was filtered over solka floc, and the cake was washed thoroughly with isopropyl acetate. The filtrate was partially concentrated to approximately 6 L, and was added hydrogen chloride in isopropyl acetate (5-6 N) while maintaining the reaction temperature at 18-25° C. After aging overnight, the precipitate was collected by filtration and washed twice with isopropyl acetate (1 L×2). The precipitate was suspended in isopropyl acetate (5.8 L) and was added aqueous potassium carbonate (1 M, 3.5 L). After stirring for 20 min, the organic layer was separated and concentrated to an oil, which was purified by preparative HPLC eluting on a Chiralpak AD column with heptane/ethanol/diethylamine (70/30/0.1; flow rate: 700 ml./min) to afford N-[3-(4-chlorophenyl)-2(S)-(3-cyanophenyl)-1(S)-methylpropyl]amine. The free amine thus obtained can be used directly or can be converted to the corresponding hydrochloride salt by treatment with hydrogen chloride in dioxane (4 N). LC-MS: m/e 285 (M+H)⁺ (2.2 min).

### Reference Example 85

### N-(3-(4-chlorophenyl)-2(S)-(3-cyanophenyl)-1(S)-methylpropyl)-2-bromo-2-methyl-propanamide

Anhydrous DMF (8.0 mL) and N-methylmorpholine (6.31 mmol, 0.695 μL) were added to a flask containing N-(3-(4-chlorophenyl)-2(S)-(3-cyanophenyl)-1(S)-methylpropylamine from Reference Example 84 (505 mg, 1.58 mmol), 2-bromo-2-methylpropionic acid (2.37 mmol, 395 mg) and PYBOP (2.37 mmol, 1.23 g). The resultant yellow solution was stirred overnight at ambient temperature. After 15 hr, DMF was removed in vacuo and the residue partitioned between EtOAc (30 mL) and H₂O (2×10 mL). The layers were separated and the organic layer washed with H₂O (2×10 mL), brine (1×10 mL) and dried over MgSO₄. The mixture was filtered and concentrated in vacuo. Purification of the residue by flash chromatography (Biotage™ 40S+ column, 10, 15, 20% EtOAc/hexanes as eluent) afforded 636 mg of the title compound as a white foam. ¹H NMR (CDCl₃) δ: 1.03 (d, 3H, J=6.6 Hz), 1.95 (s, 3H), 2.00 (s, 3H), 2.85-2.90 (m, 1H), 3.06-3.13 (m, 2H), 4.24-4.28 (m, 1H), 6.45 (d, 1H, J=8.5 Hz), 6.88 (d, 2H, J=8.2 Hz), 7.13 (d, 2H, J=8.2 Hz), 7.33 (d, 1H, J=8.0 Hz), 7.38 (d, 1H, J=2.0 Hz), 7.40 (d, 1H, J=7.7 Hz), 7.52 (d, 1H, J=7.8 Hz); HPLC rt=3.88 min, m/z=434.9 (M+H)⁺.

### Reference Example 86

### N-(3-(4-chlorophenyl)-2(S)-(3-cyanophenyl)-1(S)-methylpropyl)-2-hydroxy-2-methyl-propanamide

Anhydrous DMF (8.0 mL) and N-methylmorpholine (6.31 mmol, 0.695 μL) were added to a flask containing N-(3-(4-chlorophenyl)-2(S)-(3-cyanophenyl)-1(S)-methylpropylamine from Reference Example 84 (506 mg, 1.58 mmol), 2-hydroxy-2-methylpropionic acid (2.37 mmol, 247 mg) and PYBOP (2.37 mmol, 1.23 g). The resultant yellow solution was stirred overnight at ambient temperature. After 15 hr, DMF was removed in vacuo and the residue partitioned between EtOAc (30 mL) and H₂O (2×10 mL). The layers were separated and the organic layer washed with H₂O (2×10 mL), brine (1×10 mL) and dried over MgSO₄. The mixture was filtered and concentrated in vacuo. Purification of the residue by flash chromatography (Biotage™ 40S+ column, 20, 30, 40% EtOAc/hexanes as eluent) afforded the title compound as a white foam. ¹H NMR (CDCl₃) δ 1.02 (d, 3H, J=6.9 Hz), 1.49 (s, 3H), 1.52 (s, 3H), 2.04 (brs 1H), 2.88-2.90 (m, 1H), 3.06-3.13 (m, 2H), 4.27-4.31 (m, 1H), 6.58 (d, 1H, J=8.9 Hz), 6.90 (d, 2H, J=8.2 Hz), 7.14 (d, 2H, J=8.2 Hz), 7.33 (d, 1H, J=7.8 Hz), 7.37 (d, 1H, J=2.3 Hz), 7.40 (d, 1H, J=7.5 Hz), 7.51 (d, 1H, J=7.5 Hz); HPLC rt=3.38 min, m/z=371.1 (M+H)⁺.

### Example 1

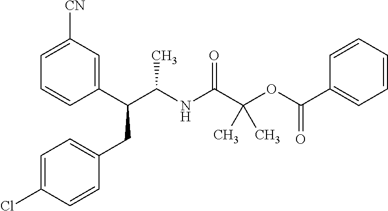

N-(3-(4-chlorophenyl)-2(S)-(3-cyanophenyl)-1(S)-methylpropyl)-2-benzoyloxy-2-methyl-propanamide (Method A) In a sealed tube, triethylamine (32.0 μL, 0.242 mmol), benzoyl chloride (28.0 μL, 0.242 mmol) and a crystal of N,N-dimethylaminopyridine were added to a solution of N-(3-(4-chlorophenyl)-2(S)-(3-cyanophenyl)-1(S)-methylpropyl)-2-hydroxy-2-methyl-propanamide from Reference Example 86 (45 mg; 0.121 mmol) in acetonitrile (1.0 mL). The reaction mixture was heated to reflux for 16 hr and cooled to ambient temperature. Excess benzoyl chloride was quenched with methanol and the reaction mixture was concentrated in vacuo. Purification of the residue by flash chromatography (Biotage™ 12S column, 20% EtOAc/hexanes as eluent) afforded 30 mg of the title compound as a white film: ¹H NMR (CD₃OD) δ 0.89 (d, 3H, J=6.6 Hz), 1.73 (s, 3H), 1.74 (s, 3H), 2.75 (dd, 1H, J=11.7, 13.5 Hz), 2.94 (ddd, 1H J=3.6, 10.3, 13.5 Hz), 3.25 (dd, 1H, J=3.7, 13.7 Hz), 4.26-4.31 (m, 1H), 6.80 (d, 2H, J=8.2 Hz), 7.04 (d, 2H, J=8.2 Hz), 7.37 (d, 2H, J=6.1 Hz), 7.42 (s, 1H), 7.50 (d, 2H, J=7.6 Hz), 7.63 (t, 1H, J=7.0 Hz), 7.89 (d, 1H, J=9.0 Hz), 8.05 (d, 2H, J=8.0 Hz); HPLC rt=3.99 min, m/z=475 (M+H)⁺.

### Example 2

### N-(3-(4-chlorophenyl)-2(S)-(3-cyanophenyl)-1(S)-methylpropyl)-2-benzoyloxy-2-methyl-propanamide (Method B)

To a solution of N-(3-(4-chlorophenyl)-2(S)-(3-cyanophenyl)-1(S)-methylpropyl)-2-bromo-2-methyl-propanamide from Reference Example 85 (100 mg, 0.230 mmol) in acetonitrile (1.75 mL), benzoic acid (28.0 mg, 0.230 mmol) and silver(I)oxide (53.0 mg, 0.230 mmol) were added and the resultant mixture was stirred at ambient temperature overnight. After 15 hr, the mixture was filtered, concentrated in vacuo and purified by flash chromatography (Biotage™ 12S column, 20% EtOAc/hexanes as eluent) to afford 101 mg of N-(3-(4-chlorophenyl)-2(S)-(3-cyanophenyl)-1(S)-methylpropyl)-2-benzoyloxy-2-methyl-propanamide as a white foam. This material had identical spectral characteristics to those reported in Example 1.

The following examples (Table 1) were prepared according to the general procedures outlined in Example 1 (Method A) or Example 2 (Method B).

TABLE 1

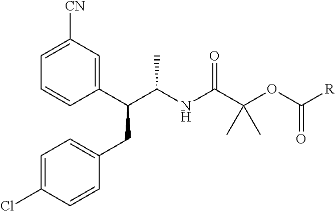

| | | | Method | | |
|---|---|---|---|---|---|
| Example | | | of | LC/MS^{a} | RT^{b} |
| No. | Name | R | Preparation | (M + H)⁺ | (min) |
| 3 | N-(3-(4-chlorophenyl)-2(S)-(3- cyanophenyl)-1(S)-methylpropyl)-2- acetoxy-2-methyl-propanamide | 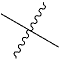 | A | 413 | 3.63 |
| 4 | N-(3-(4-chlorophenyl)-2(S)-(3- cyanophenyl)-1(S)-methylpropyl)-2- nicotinoyloxy-2-methyl- propanamide | 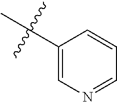 | A | 476 | 3.42 |
| 5 | N-(3-(4-chlorophenyl)-2(S)-(3- cyanophenyl)-1(S)-methylpropyl)-2- picolinoyloxy-2-methyl- propanamide | 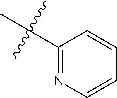 | A | 476 | 3.60 |
| 6 | N-(3-(4-chlorophenyl)-2(S)-(3- cyanophenyl)-1(S)-methylpropyl)-2- isonicotinoyloxy-2-methyl- propanamide | 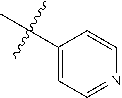 | A | 476 | 3.41 |
| 7 | N-(3-(4-chlorophenyl)-2(S)-(3- cyanophenyl)-1(S)-methylpropyl)-2- isobutanoyloxy-2-methyl- propanamide | 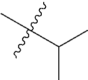 | A | 441 | 3.88 |
| 8 | N-(3-(4-chlorophenyl)-2(S)-(3- cyanophenyl)-1(S)-methylpropyl)-2- cyclohexylcarbonyloxy-2-methyl- propanamide | 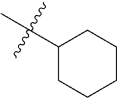 | A | 481 | 4.15 |
| 9 | N-(3-(4-chlorophenyl)-2(S)-(3- cyanophenyl)-1(S)-methylpropyl)-2- cyclopentylcarbonyloxy-2-methyl- propanamide | 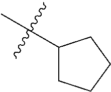 | A | 467 | 4.02 |
| 10 | N-(3-(4-chlorophenyl)-2(S)-(3- cyanophenyl)-1(S)-methylpropyl)-2- (4-cyano-benzoyloxy)-2-methyl- propanamide | 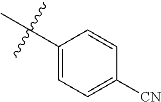 | A | 500 | 3.90 |
| 11 | N-(3-(4-chlorophenyl)-2(S)-(3- cyanophenyl)-1(S)-methylpropyl)-2- (2-trifluoromethoxy-benzoyloxy)-2- methyl-propanamide | 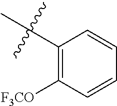 | A | 559 | 4.10 |
| 12 | N-(3-(4-chlorophenyl)-2(S)-(3- cyanophenyl)-1(S)-methylpropyl)-2- (2-chloro-benzoyloxy)-2-methyl- propanamide | 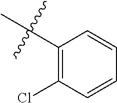 | A | 509 | 4.02 |
| 13 | N-(3-(4-chlorophenyl)-2(S)-(3- cyanophenyl)-1(S)-methylpropyl)-2- (4-isopropyl-1,2,3-thiadiazolyl-5- carbonyloxy)-2-methyl-propanamide | 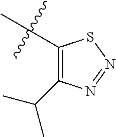 | A | 525 | 4.07 |
| 14 | N-(3-(4-chlorophenyl)-2(S)-(3- cyanophenyl)-1(S)-methylpropyl)-2- (2-hydroxy-benzoyloxy)-2-methyl- propanamide | 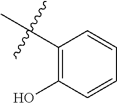 | B | 491 | 4.02 |
| 15 | N-(3-(4-chlorophenyl)-2(S)-(3- cyanophenyl)-1(S)-methylpropyl)-2- (5-trifluoromethyl-1-phenyl- pyrazolyl-4-carbonyloxy)-2-methyl- propanamide | 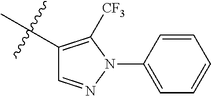 | B | 609 | 4.11 |
| 16 | N-(3-(4-chlorophenyl)-2(S)-(3- cyanophenyl)-1(S)-methylpropyl)-2- (2-fluoro-benzoyloxy)-2-methyl- propanamide | 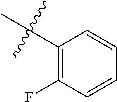 | A | 493 | 3.84 |
| 17 | N-(3-(4-chlorophenyl)-2(S)-(3- cyanophenyl)-1(S)-methylpropyl)-2- (1,3-dimethyl-pyrazolyl-5- carbonyloxy)-2-methyl-propanamide | 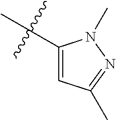 | A | 493 | 3.78 |
| 18 | N-(3-(4-chlorophenyl)-2(S)-(3- cyanophenyl)-1(S)-methylpropyl)-2- pyrazinylcarbonyloxy-2-methyl- propanamide | 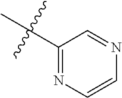 | B | 477 | 3.49 |
| 19 | N-(3-(4-chlorophenyl)-2(S)-(3- cyanophenyl)-1(S)-methylpropyl)-2- (3-phenyl-isoxazolyl-5- carbonyloxy)-2-methyl-propanamide | 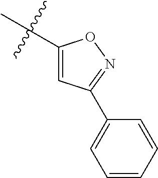 | B | 542 | 4.07 |
| 20 | N-(3-(4-chlorophenyl)-2(S)-(3- cyanophenyl)-1(S)-methylpropyl)-2- (pyrazolo[1,5-a]pyrimidinyl-3- carbonyloxy)-2-methyl-propanamide | 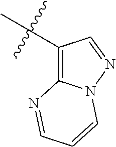 | B | 516 | 3.66 |
| 21 | N-(3-(4-chlorophenyl)-2(S)-(3- cyanophenyl)-1(S)-methylpropyl)-2- ((2R)-(acetoxy)(phenyl)acetoxy)-2- methyl-propanamide | 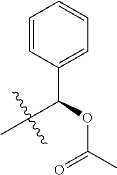 | B | 487 | 4.01 |
| 22 | N-(3-(4-chlorophenyl)-2(S)-(3- cyanophenyl)-1(S)-methylpropyl)-2- (2-acetoxy-acetoxy)-2-methyl- propanamide | 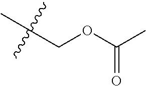 | A | | |
| 23 | N-(3-(4-chlorophenyl)-2(S)-(3- cyanophenyl)-1(S)-methylpropyl)-2- ((2S)-(hydroxy)(phenyl)acetoxy)-2- methyl-propanamide | 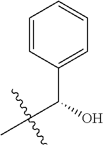 | B | 505 | 3.72 |
| 24 | N-(3-(4-chlorophenyl)-2(S)-(3- cyanophenyl)-1(S)-methylpropyl)-2- (5-methyl-pyrazinylcarbonyloxy)-2- methyl-propanamide | 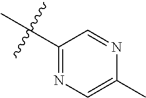 | B | 491 | 3.58 |
| 25 | N-(3-(4-chlorophenyl)-2(S)-(3- cyanophenyl)-1(S)-methylpropyl)-2- (4-phenyl-1,2,3-thiadiazolyl-5- carbonyloxy)-2-methyl-propanamide | 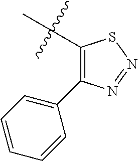 | B | 559 | 4.06 |
| 26 | N-(3-(4-chlorophenyl)-2(S)-(3- cyanophenyl)-1(S)-methylpropyl)-2- (isoxazolyl-5-carbonyloxy)-2- methyl-propanamide | 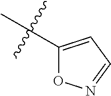 | B | 466 | 3.71 |
| 27 | N-(3-(4-chlorophenyl)-2(S)-(3- cyanophenyl)-1(S)-methylpropyl)-2- (3-tert-butyl-1-methyl-pyrazolyl-5- carbonyloxy)-2-methyl-propanamide | 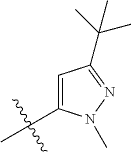 | B | 535 | 4.14 |
| 28 | N-(3-(4-chlorophenyl)-2(S)-(3- cyanophenyl)-1(S)-methylpropyl)-2- (1-tert-butyl-3-methyl-pyrazolyl-5- carbonyloxy)-2-methyl-propanamide | 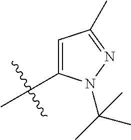 | B | 535 | 3.98 |
| 29 | N-(3-(4-chlorophenyl)-2(S)-(3- cyanophenyl)-1(S)-methylpropyl)-2- (5-methyl-isoxazolyl-3- carbonyloxy)-2-methyl-propanamide | 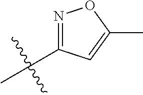 | B | 480 | 3.79 |
| 30 | N-(3-(4-chlorophenyl)-2(S)-(3- cyanophenyl)-1(S)-methylpropyl)-2- (2,6-dichloro-benzoyloxy)-2-methyl- propanamide | 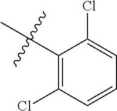 | B | 543 | 4.14 |
| 31 | N-(3-(4-chlorophenyl)-2(S)-(3- cyanophenyl)-1(S)-methylpropyl)-2- (2,6-difluoro-benzoyloxy)-2-methyl- propanamide | 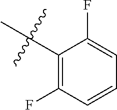 | B | 511 | 3.97 |
| 32 | N-(3-(4-chlorophenyl)-2(S)-(3- cyanophenyl)-1(S)-methylpropyl)-2- (4-acetoxy-benzoyloxy)-2-methyl- propanamide | 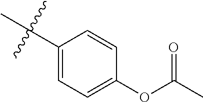 | B | 533 | 3.92 |
| 33 | N-(3-(4-chlorophenyl)-2(S)-(3- cyanophenyl)-1(S)-methylpropyl)-2- (3,5-dimethyl-isoxazolyl-4- carbonyloxy)-2-methyl-propanamide | 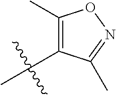 | B | 494 | 3.84 |
| 34 | N-(3-(4-chlorophenyl)-2(S)-(3- cyanophenyl)-1(S)-methylpropyl)-2- (1,5-dimethyl-pyrazolyl-3- carbonyloxy)-2-methyl-propanamide | 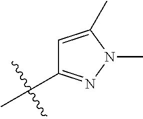 | B | 493 | 3.78 |
| 35 | N-(3-(4-chlorophenyl)-2(S)-(3- cyanophenyl)-1(S)-methylpropyl)-2- (4-hydroxy-benzoyloxy)-2-methyl- propanamide | 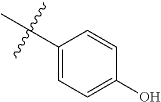 | B | 491 | 3.68 |
| 36 | N-(3-(4-chlorophenyl)-2(S)-(3- cyanophenyl)-1(S)-methylpropyl)-2- (1-methyl-5-trifluoromethyl- pyrazolyl-4-carbonyloxy)-2-methyl- propanamide | 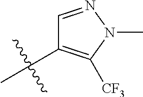 | B | 547 | 3.84 |
| 37 | N-(3-(4-chlorophenyl)-2(S)-(3- cyanophenyl)-1(S)-methylpropyl)-2- ((2S)-(acetoxy)(phenyl)acetoxy)-2- methyl-propanamide | 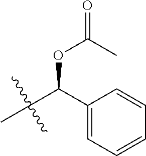 | B | 547 | 3.97 |
| 38 | N-(3-(4-chlorophenyl)-2(S)-(3- cyanophenyl)-1(S)-methylpropyl)-2- ((2R)-(hydroxy)(phenyl)acetoxy)-2- methyl-propanamide | 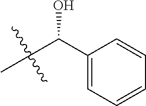 | B | 505 | 3.71 |
| 39 | N-(3-(4-chlorophenyl)-2(S)-(3- cyanophenyl)-1(S)-methylpropyl)-2- (3-acetoxy-benzoyloxy)-2-methyl- propanamide | 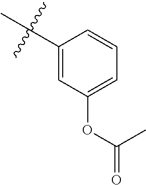 | B | 533 | 3.92 |
| 40 | N-(3-(4-chlorophenyl)-2(S)-(3- cyanophenyl)-1(S)-methylpropyl)-2- (3-hydroxy-benzoyloxy)-2-methyl- propanamide | 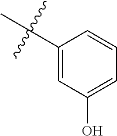 | B | 491 | 3.71 |
| 41 | N-(3-(4-chlorophenyl)-2(S)-(3- cyanophenyl)-1(S)-methylpropyl)-2- (2,4-dimethyl-thiazolyl-5- carbonyloxy)-2-methyl-propanamide | 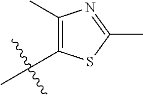 | B | 510 | 3.80 |
| 42 | N-(3-(4-chlorophenyl)-2(S)-(3- cyanophenyl)-1(S)-methylpropyl)-2- (1-methyl-3-trifluoromethyl- pyrazolyl-5-carbonyloxy)-2-methyl- propanamide | 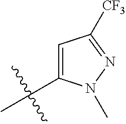 | B | 547 | 4.04 |
| 43 | N-(3-(4-chlorophenyl)-2(S)-(3- cyanophenyl)-1(S)-methylpropyl)-2- (pyrimidinyl-5-carbonyloxy)-2- methyl-propanamide | 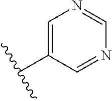 | B | 477 | 3.56 |
| 44 | N-(3-(4-chlorophenyl)-2(S)-(3- cyanophenyl)-1(S)-methylpropyl)-2- (2-methyl-5-trifluoromethyl- oxazolyl-3-carbonyloxy)-2-methyl- propanamide | 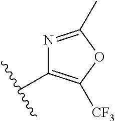 | B | 548 | 3.89 |
| 45 | N-(3-(4-chlorophenyl)-2(S)-(3- cyanophenyl)-1(S)-methylpropyl)-2- phenylacetoxy-2-methyl- propanamide | 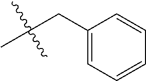 | B | 489 | 3.96 |
| 46 | N-(3-(4-chlorophenyl)-2(S)-(3- cyanophenyl)-1(S)-methylpropyl)-2- ((2R)-(tert-butyloxycarbonylamino)- (phenyl)acetoxy)-2-methyl- propanamide | 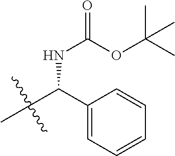 | B | 604 | 4.16 |
| 47 | N-(3-(4-chlorophenyl)-2(S)-(3- cyanophenyl)-1(S)-methylpropyl)-2- ((2S)-(tert-butyloxycarbonylamino)- (phenyl)acetoxy)-2-methyl- propanamide | 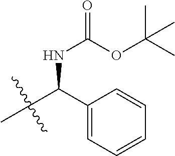 | B | 604 | 4.16 |
| 48 | N-(3-(4-chlorophenyl)-2(S)-(3- cyanophenyl)-1(S)-methylpropyl)-2- (5-methyl-3-phenyl-isoxazolyl-4- carbonyloxy)-2-methyl-propanamide | 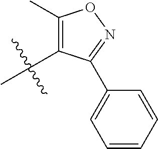 | B | 556 | 4.02 |
| 49 | N-(3-(4-chlorophenyl)-2(S)-(3- cyanophenyl)-1(S)-methylpropyl)-2- (1-(4-chlorophenyl)-5- trifluoromethyl-pyrazolyl-4- carbonyloxy)-2-methyl-propanamide | 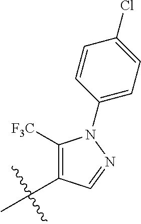 | B | 643 | 4.25 |
| 50 | N-(3-(4-chlorophenyl)-2(S)-(3- cyanophenyl)-1(S)-methylpropyl)-2- (1-ethyl-3-methyl-pyrazolyl-5- carbonyloxy)-2-methyl-propanamide | 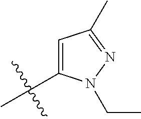 | B | 507 | 3.88 |
| 51 | N-(3-(4-chlorophenyl)-2(S)-(3- cyanophenyl)-1(S)-methylpropyl)-2- (1,3,5-trimethyl-pyrazolyl-4- carbonyloxy)-2-methyl-propanamide | 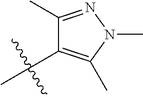 | B | 507 | 3.67 |
| 52 | N-(3-(4-chlorophenyl)-2(S)-(3- cyanophenyl)-1(S)-methylpropyl)-2- (6-methyl-imidazo[2,1-b]thiazolyl-5- carbonyloxy)-2-methyl-propanamide | 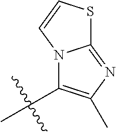 | B | 535 | 3.62 |
| 53 | N-(3-(4-chlorophenyl)-2(S)-(3- cyanophenyl)-1(S)-methylpropyl)-2- (2-(2-pyridyl)acetoxy)-2-methyl- propanamide | 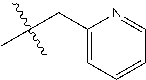 | B | 490 | 3.02 |
| 54 | N-(3-(4-chlorophenyl)-2(S)-(3- cyanophenyl)-1(S)-methylpropyl)-2- (1-phenyl-pyrazolyl-4-carbonyloxy)- 2-methyl-propanamide | 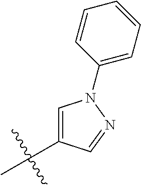 | B | 541 | 3.99 |
| 55 | N-(3-(4-chlorophenyl)-2(S)-(3- cyanophenyl)-1(S)-methylpropyl)-2- (2-methyl-imidazo[1,2-a]pyridyl-4- carbonyloxy)-2-methyl-propanamide | 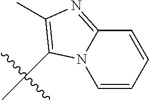 | B | 529 | 3.26 |
| 56 | N-(3-(4-chlorophenyl)-2(S)-(3- cyanophenyl)-1(S)-methylpropyl)-2- (2-phenyl-5-trifluoromethyl- oxazolyl-4-carbonyloxy)-2-methyl- propanamide | 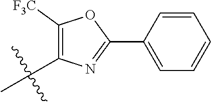 | B | 610 | 4.22 |
| 57 | N-(3-(4-chlorophenyl)-2(S)-(3- cyanophenyl)-1(S)-methylpropyl)-2- (1-methyl-imidazolyl-2- carbonyloxy)-2-methyl-propanamide | 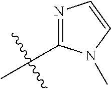 | B | 479 | 3.20 |
| 58 | N-(3-(4-chlorophenyl)-2(S)-(3- cyanophenyl)-1(S)-methylpropyl)-2- (1-methyl-imidazolyl-5- carbonyloxy)-2-methyl-propanamide | 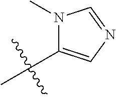 | B | 479 | 3.12 |
| 59 | N-(3-(4-chlorophenyl)-2(S)-(3- cyanophenyl)-1(S)-methylpropyl)-2- (pyrazolo[1,5-a]pyridyl-3- carbonyloxy)-2-methyl-propanamide | 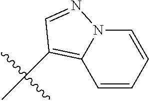 | B | 515 | 3.76 |
| 60 | N-(3-(4-chlorophenyl)-2(S)-(3- cyanophenyl)-1(S)-methylpropyl)-2- (1-isopropyl-pyrazolyl-4- carbonyloxy)-2-methyl-propanamide | 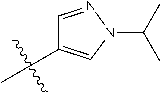 | B | 507 | 3.77 |
| 61 | N-(3-(4-chlorophenyl)-2(S)-(3- cyanophenyl)-1(S)-methylpropyl)-2- (1-methyl-imidazolyl-4- carbonyloxy)-2-methyl-propanamide | 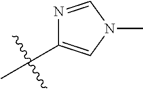 | B | 479 | 3.12 |
| 62 | N-(3-(4-chlorophenyl)-2(S)-(3- cyanophenyl)-1(S)-methylpropyl)-2- (4-methyl-1,2,5-oxadiazolyl-3- carbonyloxy)-2-methyl-propanamide | 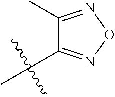 | B | 481 | 3.89 |
| 63 | N-(3-(4-chlorophenyl)-2(S)-(3- cyanophenyl)-1(S)-methylpropyl)-2- (5-methyl-2-phenyl-2H-1,2,3- triazolyl-4-carbonyloxy)-2-methyl- propanamide | 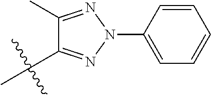 | B | 556 | 4.16 |
| 64 | N-(3-(4-chlorophenyl)-2(S)-(3- cyanophenyl)-1(S)-methylpropyl)-2- (5-isopropyl-isoxazolyl-3- carbonyloxy)-2-methyl-propanamide | 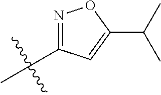 | B | 508 | 4.01 |
| 65 | N-(3-(4-chlorophenyl)-2(S)-(3- cyanophenyl)-1(S)-methylpropyl)-2- (3-methyl-5-(4-methyl-1,2,3-thiazol- 5-yl)isoxazolyl-4-carbonyloxy)-2- methyl-propanamide | 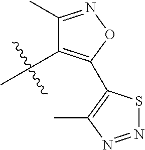 | B | 578 | 3.93 |
| 66 | N-(3-(4-chlorophenyl)-2(S)-(3- cyanophenyl)-1(S)-methylpropyl)-2- (1-methyl-pyrazolyl-4-carbonyloxy)- 2-methyl-propanamide | 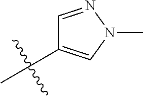 | B | 479 | 3.56 |
| 67 | N-(3-(4-chlorophenyl)-2(S)-(3- cyanophenyl)-1(S)-methylpropyl)-2- (5-cyclopropyl-isoxazolyl-3- carbonyloxy)-2-methyl-propanamide | 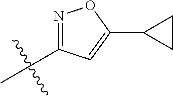 | B | 506 | 3.92 |
| 68 | N-(3-(4-chlorophenyl)-2(S)-(3- cyanophenyl)-1(S)-methylpropyl)-2- (3-methyl-5-phenyl-isoxazolyl-4- carbonyloxy)-2-methyl-propanamide | 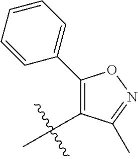 | B | 556 | 4.10 |
| 69 | N-(3-(4-chlorophenyl)-2(S)-(3- cyanophenyl)-1(S)-methylpropyl)-2- (1-methyl-3-trifluoromethyl-1H- pyrazolyl-4-carbonyloxy)-2-methyl- propanamide | 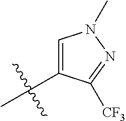 | B | 547 | 3.84 |
| 70 | N-(3-(4-chlorophenyl)-2(S)-(3- cyanophenyl)-1(S)-methylpropyl)-2- (5-phenyl-1,2,4-oxadiazolyl-3- carbonyloxy)-2-methyl-propanamide | 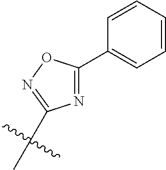 | B | 543 | 4.02 |
| 71 | N-(3-(4-chlorophenyl)-2(S)-(3- cyanophenyl)-1(S)-methylpropyl)-2- (2-acetoxy-benzoyloxy)-2-methyl- propanamide | 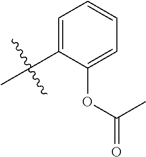 | B | 533 | 3.91 |
| 72 | N-(3-(4-chlorophenyl)-2(S)-(3- cyanophenyl)-1(S)-methylpropyl)-2- (2-methyl-benzoyloxy)-2-methyl- propanamide | 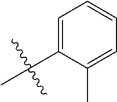 | B | 489 | 4.08 |
| 73 | N-(3-(4-chlorophenyl)-2(S)-(3- cyanophenyl)-1(S)-methylpropyl)-2- (5-phenyl-isoxazolyl-3- carbonyloxy)-2-methyl-propanamide | 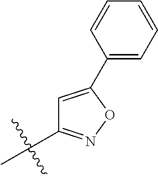 | B | 542 | 4.11 |
| 74 | N-(3-(4-chlorophenyl)-2(S)-(3- cyanophenyl)-1(S)-methylpropyl)-2- (pivaloyloxy)-2-methyl-propanamide | 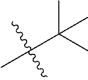 | B | 455 | 4.01 |
| 75 | N-(3-(4-chlorophenyl)-2(S)-(3- cyanophenyl)-1(S)-methylpropyl)-2- (2-ethyl-benzoyloxy)-2-methyl- propanamide | 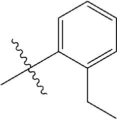 | B | 503 | 4.18 |
| 76 | N-(3-(4-chlorophenyl)-2(S)-(3- cyanophenyl)-1(S)-methylpropyl)-2- (2,6-dimethyl-benzoyloxy)-2- methyl-propanamide | 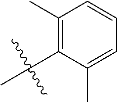 | B | 503 | 4.14 |
| 77 | N-(3-(4-chlorophenyl)-2(S)-(3- cyanophenyl)-1(S)-methylpropyl)-2- (2,4-dichloro-benzoyloxy)-2-methyl- propanamide | 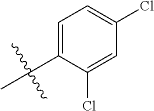 | B | 543 | 4.23 |
| 78 | N-(3-(4-chlorophenyl)-2(S)-(3- cyanophenyl)-1(S)-methylpropyl)-2- (4-fluoro-2-methyl-benzoyloxy)-2- methyl-propanamide | 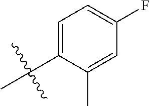 | B | 507 | 4.11 |
| 79 | N-(3-(4-chlorophenyl)-2(S)-(3- cyanophenyl)-1(S)-methylpropyl)-2- (4-fluoro-2-trifluoromethyl- benzoyloxy)-2-methyl-propanamide | 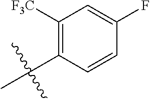 | B | 543 | 4.12 |
| 80 | N-(3-(4-chlorophenyl)-2(S)-(3- cyanophenyl)-1(S)-methylpropyl)-2- (2-trifluoromethyl-benzoyloxy)-2- methyl-propanamide | 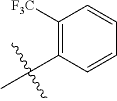 | B | 543 | 4.08 |
| 81 | N-(3-(4-chlorophenyl)-2(S)-(3- cyanophenyl)-1(S)-methylpropyl)-2- (2-methoxy-benzoyloxy)-2-methyl- propanamide | 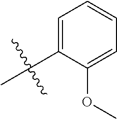 | B | 505 | 3.80 |
| 82 | N-(3-(4-chlorophenyl)-2(S)-(3- cyanophenyl)-1(S)-methylpropyl)-2- (2,6-dimethoxy-benzoyloxy)-2- methyl-propanamide | 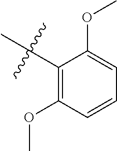 | B | 535 | 3.95 |
| 83 | N-(3-(4-chlorophenyl)-2(S)-(3- cyanophenyl)-1(S)-methylpropyl)-2- (3-cyano-benzoyloxy)-2-methyl- propanamide | 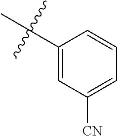 | B | 500 | 3.89 |
| 84 | N-(3-(4-chlorophenyl)-2(S)-(3- cyanophenyl)-1(S)-methylpropyl)-2- (2-chloro-6-fluoro-benzoyloxy)-2- methyl-propanamide | 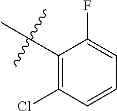 | B | 527 | 4.06 |
| 85 | N-(3-(4-chlorophenyl)-2(S)-(3- cyanophenyl)-1(S)-methylpropyl)-2- (4-trifluoromethoxy-benzoyloxy)-2- methyl-propanamide | 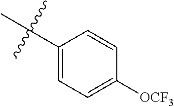 | B | 559 | 4.19 |
| 86 | N-(3-(4-chlorophenyl)-2(S)-(3- cyanophenyl)-1(S)-methylpropyl)-2- (tetrahydrofuranyl-2(S)- carbonyloxy)-2-methyl-propanamide | 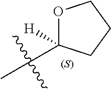 | B | 469 | 3.67 |
| 87 | N-(3-(4-chlorophenyl)-2(S)-(3- cyanophenyl)-1(S)-methylpropyl)-2- (2-acetamido-6-fluoro-benzoyloxy)- 2-methyl-propanamide | 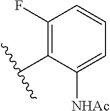 | B | 550 | 3.93 |
| 88 | N-(3-(4-chlorophenyl)-2(S)-(3- cyanophenyl)-1(S)-methylpropyl)-2- (2-amino-benzoyloxy)-2-methyl- propanamide | 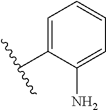 | B | 490 | 3.90 |
| 89 | N-(3-(4-chlorophenyl)-2(S)-(3- cyanophenyl)-1(S)-methylpropyl)-2- (5-oxo-tetrahydrofuranyl-2(R)- carbonyloxy)-2-methyl-propanamide | 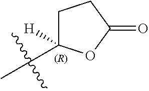 | B | 483 | 3.57 |
| 90 | N-(3-(4-chlorophenyl)-2(S)-(3- cyanophenyl)-1(S)-methylpropyl)-2- (5-oxo-tetrahydrofuranyl-2(S)- carbonyloxy)-2-methyl-propanamide | 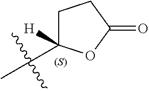 | B | 483 | 3.58 |
| 91 | N-(3-(4-chlorophenyl)-2(S)-(3- cyanophenyl)-1(S)-methylpropyl)-2- (4-chloro-benzoyloxy)-2-methyl- propanamide | 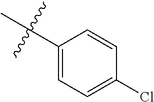 | B | 509 | 4.13 |
| 92 | N-(3-(4-chlorophenyl)-2(S)-(3- cyanophenyl)-1(S)-methylpropyl)-2- (6-chloro-2-fluoro-3-methyl- benzoyloxy)-2-methyl-propanamide | 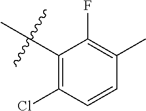 | B | 541 | 4.16 |
| 93 | N-(3-(4-chlorophenyl)-2(S)-(3- cyanophenyl)-1(S)-methylpropyl)-2- (3,3-dimethyl-butyryloxy)-2-methyl- propanamide | 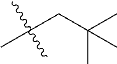 | B | 469 | 4.09 |
| 94 | N-(3-(4-chlorophenyl)-2(S)-(3- cyanophenyl)-1(S)-methylpropyl)-2- anilino-2-methyl-propanamide | 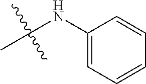 | B | 490 | 3.85 |
| 95 | N-(3-(4-chlorophenyl)-2(S)-(3- cyanophenyl)-1(S)-methylpropyl)-2- (3,4-dichloro-benzoyloxy)-2-methyl- propanamide | 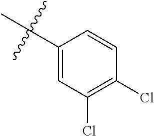 | B | 543 | 4.26 |
| 96 | N-(3-(4-chlorophenyl)-2(S)-(3- cyanophenyl)-1(S)-methylpropyl)-2- (2-chloro-3,6-dichloro-benzoyloxy)- 2-methyl-propanamide | 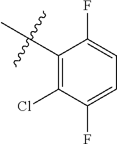 | B | 545 | 4.07 |
| 97 | N-(3-(4-chlorophenyl)-2(S)-(3- cyanophenyl)-1(S)-methylpropyl)-2- (2-chloro-6-fluoro-3-methyl- benzoyloxy)-2-methyl-propanamide | 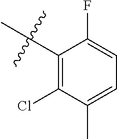 | B | 541 | 4.14 |
| 98 | N-(3-(4-chlorophenyl)-2(S)-(3- cyanophenyl)-1(S)-methylpropyl)-2- (2-amino-6-chloro-benzoyloxy)-2- methyl-propanamide | 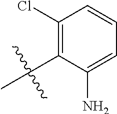 | B | 524 | 3.85 |
| 99 | N-(3-(4-chlorophenyl)-2(S)-(3- cyanophenyl)-1(S)-methylpropyl)-2- (2-chloro-6-methyl-benzoyloxy)-2- methyl-propanamide | 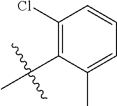 | B | 524 | 4.00 |
| 100 | N-(3-(4-chlorophenyl)-2(S)-(3- cyanophenyl)-1(S)-methylpropyl)-2- (2-phenyl-tetrahydrofuranyl-2- carbonyloxy)-2-methyl-propanamide | 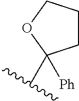 | B | 545 | 4.07 |
| 101 | N-(3-(4-chlorophenyl)-2(S)-(3- cyanophenyl)-1(S)-methylpropyl)-2- (2-methyl-tetrahydrofuranyl-2- carbonyloxy)-2-methyl-propanamide | 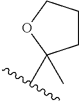 | B | 505 | 3.77 |
| 102 | N-(3-(4-chlorophenyl)-2(S)-(3- cyanophenyl)-1(S)-methylpropyl)-2- (2-methyl-5-oxo-tetrahydrofuranyl- 2-carbonyloxy)-2-methyl- propanamide | 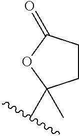 | B | 497 | 3.56 |
| 103 | N-(3-(4-chlorophenyl)-2(S)-(3- cyanophenyl)-1(S)-methylpropyl)-2- (6-methyl-2-trifluoroacetamido- benzoyloxy)-2-methyl-propanamide | 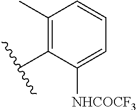 | B | 600 | 4.16 |
| 104 | N-(3-(4-chlorophenyl)-2(S)-(3- cyanophenyl)-1(S)-methylpropyl)-2- (2-amino-6-fluoro-benzoyloxy)-2- methyl-propanamide | 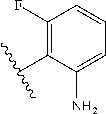 | B | 508 | 3.88 |
| 105 | N-(3-(4-chlorophenyl)-2(S)-(3- cyanophenyl)-1(S)-methylpropyl)-2- (2-amino-nicotinoyloxy)-2-methyl- propanamide | 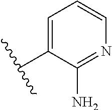 | B | 491 | 3.20 |
| 106 | N-(3-(4-chlorophenyl)-2(S)-(3- cyanophenyl)-1(S)-methylpropyl)-2- (2-fluoro-isonicotinoyloxy)-2- methyl-propanamide | 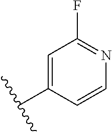 | B | 494 | 3.84 |
| 107 | N-(3-(4-chlorophenyl)-2(S)-(3- cyanophenyl)-1(S)-methylpropyl)-2- (2-(2,2,2-trifluoroethoxy)- benzoyloxy)-2-methyl-propanamide | 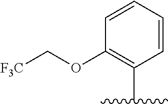 | B | 4.02 | 573 |
| 108 | N-(3-(4-chlorophenyl)-2(S)-(3- cyanophenyl)-1(S)-methylpropyl)-2- (2-(1,1,2,2-tetrafluoroethyl)- benzoyloxy)-2-methyl-propanamide | 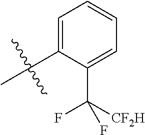 | B | 575 | 3.95 |
| 109 | N-(3-(4-chlorophenyl)-2(S)-(3- cyanophenyl)-1(S)-methylpropyl)-2- (2-acetamido-6-chloro-benzoyloxy)- 2-methyl-propanamide | 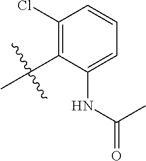 | B | 566 | 3.91 |
| 110 | N-(3-(4-chlorophenyl)-2(S)-(3- cyanophenyl)-1(S)-methylpropyl)-2- (2-chloro-6-methyl- isonicotinoyloxy)-2-methyl- propanamide | 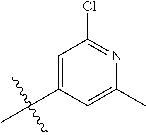 | B | 524 | 3.86 |
| 111 | N-(3-(4-chlorophenyl)-2(S)-(3- cyanophenyl)-1(S)-methylpropyl)-2- (2-amino-6-methyl-benzoyloxy)-2- methyl-propanamide | 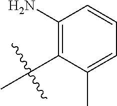 | B | 504 | 3.74 |

^{a}LC/MS (M + H) = liquid chromatography/mass spectrum (mass of parent ion plus one hydrogen)
See General Procedures for LC/MS conditions.
^{b}RT (min) = retention time in minutes from the LC/MS determination.

### Example 112

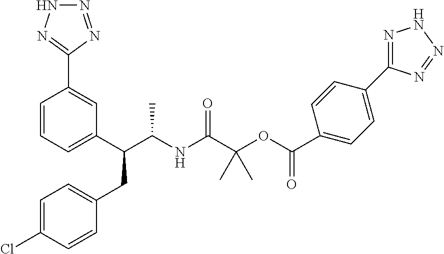

### N-(3-(4-Chlorophenyl)-2-(3-(2H-tetrazol-5-yl)phenyl)-1-methylpropyl)-2-(4-(2H-tetrazol-5-yl)benzoyloxy)-2-methyl-propanamide

To a solution of N-(3-(4-chlorophenyl)-2-(3-cyanophenyl)-1-methylpropyl)-2-(4-cyano-benzoyloxy)-2-methyl-propanamide (Example 10 in Table 1) (65 mg, 0.130 mmol) in toluene (2.0 mL), azidotrimethylsilane (170 mL, 1.30 mmol) and dibutyltin oxide (16 mg, 0.00651 mmol) were added. The reaction mixture was heated to reflux for 15 hr, cooled to ambient temperature and diluted with EtOAc. The organic layer was washed with H₂O, brine, dried over MgSO₄ and filtered. Volatiles were concentrated in vacuo and the residue purified by RP-HPLC to afford the title compound as a white film: ¹H NMR (CD₃OD) δ 0.97 (d, 3H, J=6.6 Hz), 1.79 (d, 6H, J=5.7 Hz), 2.84 (dd, 1H, J=11.5, 13.5 Hz), 2.99-3.04 (m, 1H), 3.28-3.30 (m, 1H), 4.34-4.39 (m, 1H), 6.86 (d, 2H, J=8.5 Hz), 7.02 (d, 2H, J=8.2 Hz), 7.28 (d, 1H, J=7.6 Hz), 7.43 (d, 1H, J=7.8 Hz), 7.78 (s, 1H), 7.82 (d, 1H, J=8.5 Hz), 7.98 (d, 1H, J=8.9 Hz), 8.16 (d, 2H, J=8.5 Hz), 8.23 (d, 2H, J=8.5 Hz); HPLC rt=3.39 min, m/z=586 (M+H)⁺.

### Example 113

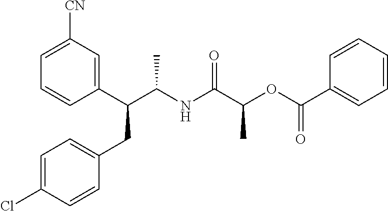

### N-(3-(4-chlorophenyl)-2(S)-(3-cyanophenyl)-1(S)-methylpropyl)-2(S)-benzoyloxy)-propanamide

### Step A N-(3-(4-chlorophenyl)-2(S)-(3-cyanophenyl)-1(S)-methylpropyl)-2(S)-hydroxy)-propanamide

To a solution of N-[3-(4-chlorophenyl)-2(S)-(3-cyanophenyl)-1(S)-methylpropyl]amine hydrochloride (43.8 mg, 0.137 mmol) and (R)-lactic acid (22.0 mg, 0.205 mmol) in DMF (1.0 mL), PyBOP (107 mg, 0.205 mmol) and N-methylmorpholine (60 μL, 0.547 mmol) were added. The resultant yellow solution was stirred at ambient temperature for 15 hr. The reaction mixture was diluted with EtOAc (10 mL) and washed with H₂O (4×3 mL), Brine (1×3 mL), dried over Na₂SO₄, filtered and concentrated in vacuo. Purification of the residue by flash chromatography (Biotage™ 12S column, 20% EtOAc/hexanes as eluent) afforded 48.0 mg of the title compound as a white film: ¹H NMR (CDCl₃) δ 1.00 (d, 3H, J=6.7 Hz), 1.45 (d, 3H, J=6.9 Hz), 2.38 (d, 1H, J=4.6 Hz), 2.86 (dd, 1H, J=9.9, 13.8 Hz), 3.00-3.05 (m, 1H), 3.11 (dd, 1H, J=5.1, 13.8 Hz), 4.26-4.36 (m, 2H), 6.34 (d, 1H, J=9.2 Hz), 6.87 (d, 2H, J=8.5 Hz), 7.12 (d, 2H, J=8.2 Hz), 7.32-7.39 (m, 3H), 7.49 (d, 1H, J=7.3 Hz).

### Step B N-(3-(4-chlorophenyl)-2(S)-(3-cyanophenyl)-1(S)-methylpropyl)-2(S)-benzoyloxy)-propanamide

To a solution of the alcohol from Step A (21.0 mg, 0.0588 mmol) in toluene (1.0 mL) triethylamine (0.0883 mmol, 12.3 μL), benzoyl chloride (0.0883 mmol, 10.2 μL) and one crystal of DMAP were added. The reaction mixture was heated at 80° C. for 3 hr, cooled to ambient temperature and concentrated in vacuo. Purification of the residue by flash chromatography (Biotage™ 12S column, 5, 10, 15% acetone/hexanes as eluent) afforded the title compound as a colorless film: ¹H NMR (CDCl₃) δ 1.01 (d, 3H, J=6.9 Hz), 1.61 (d, 3H, J=6.9 Hz), 2.82 (dd, 1H, J=10.1, 13.7 Hz), 2.93-2.97 (m, 1H), 3.05 (dd, 1H, J=4.8, 13.7 Hz), 4.36-4.39 (m, 1H), 5.45 (q, 1H, J=6.9 Hz), 5.80 (d, 1H, J=9.0 Hz), 6.78 (d, 2H, J=8.2 Hz), 7.07 (d, 2H, J=8.5 Hz), 7.16-7.23 (m, 2H), 7.33 (s, 1H), 7.40 (d, 1H, J=9.0 Hz), 7.48-7.51 (m, 2H), 7.64-7.67 (m, 1H), 8.02 (d, 2H, J=9.6 Hz); HPLC rt=3.87 min, m/z=461 (M+H)⁺.

The following examples in Table 2 were prepared according to the general procedures outlined in Example 113.

TABLE 2

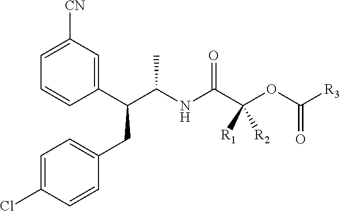

| Exp. | | | | | LC/MS^{a} | RT^{b} |
|---|---|---|---|---|---|---|
| No. | Name | R₁ | R₂ | R₃ | (M + H)⁺ | (min) |
| 114 | N-(3-(4-chlorophenyl)-2(S)-(3- cyanophenyl)-1(S)-methylpropyl)- 2(R)-benzoyloxy)-propariamide | H | CH₃ | 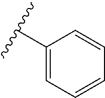 | 461 | 3.88 |
| 115 | N-(3-(4-chlorophenyl)-2(S)-(3- cyanophenyl)-1(S)-methylpropyl)- 2(S)-benzoyloxy)-butanamide | CH₂CH₃ | H | 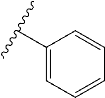 | 475 | 3.97 |
| 116 | N-(3-(4-chlorophenyl)-2(S)-(3- cyanophenyl)-1(S)-methylpropyl)- 2(R)-benzoyloxy)-butanamide | H | CH₂CH₃ | 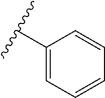 | 475 | 3.98 |
| 117 | N-(3-(4-chlorophenyl)-2(S)-(3- cyanophenyl)-1(S)-methylpropyl)- 1-benzoyloxy)-cyclopropane-1- carboxamide | CH₂— | —CH₂ | 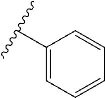 | 473 | 3.88 |
| 118 | N-(3-(4-chlorophenyl)-2(S)-(3- cyanophenyl)-1(S)-methylpropyl)- 2(S)-benzoyloxy)-3- methylbutanamide | i-Pr | H | 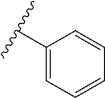 | 489 | 4.06 |
| 119 | N-(3-(4-chlorophenyl)-2(S)-(3- cyanophenyl)-1(S)-methylpropyl)- 2(R)-benzoyloxy)-3- methylbutanamide | H | i-Pr | 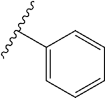 | 489 | 4.07 |
| 120 | N-(3-(4-chlorophenyl)-2(S)-(3- cyanophenyl)-1(S)-methylpropyl)- 2(S)-benzoyloxy)- phenylacetamide | 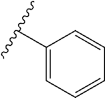 | H | 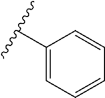 | 523 | 4.06 |
| 121 | N-(3-(4-chlorophenyl)-2(S)-(3- cyanophenyl)-1(S)-methylpropyl)- 2(R)-benzoyloxy)- phenylacetamide | H | 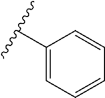 | 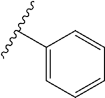 | 523 | 4.07 |
| 122 | N-(3-(4-chlorophenyl)-2(S)-(3- cyanophenyl)-1(S)-methylpropyl)- 2(S)-(2,6-dichloro-benzoyloxy)- butanamide | CH₂CH₃ | H | 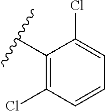 | 545 | 4.09 |
| 123 | N-(3-(4-chlorophenyl)-2(S)-(3- cyanophenyl)-1(S)-methylpropyly 2(R)-(2-ethyl-benzoyloxy)- butanamide | H | CH₂CH₃ | 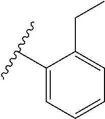 | 503 | 4.14 |
| 124 | N-(3-(4-chlorophenyl)-2(S)-(3- cyanophenyl)-1(S)-methylpropyl)- 2(R)-(2-(2,2,2-trifluoroethoxy)- benzoyloxy)-butanamide | H | CH₂CH₃ | 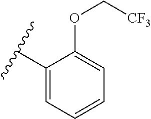 | 573 | 4.02 |
| 125 | N-(3-(4-chlorophenyl)-2(S)-(3- cyanophenyl)-1(S)-methylpropyl)- 2(R)-(cyclobutane-carbonyloxy)- 3,3-dimethyl-butanamide | H | t-Bu | 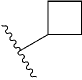 | 481 | 4.13 |
| 126 | N-(3-(4-chlorophenyl)-2(S)-(3- cyanophenyl)-1(S)-methylpropyl)- 2(R)-(cyclobutane-carbonyloxy)- 3-phenyl-propanamide | H | 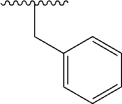 | 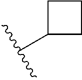 | 515 | 4.12 |
| 127 | N-(3-(4-chlorophenyl)-2(S)-(3- cyanophenyl)-1(S)-methylpropyl)- 2(R)-(3,3-dimethylbutyryloxy)-3- phenyl-propanamide | H | 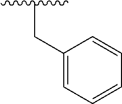 | 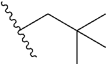 | 531 | 4.24 |
| 128 | N-(3-(4-chlorophenyl)-2(S)-(3- cyanophenyl)-1(S)-methylpropyl)- 2(R)-(2,6-dichlorobenzoyloxy)-3- methylbutanamide | H | i-Pr | 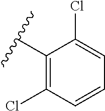 | 558 | 4.13 |
| 129 | N-(3-(4-chlorophenyl)-2(S)-(3- cyanophenyl)-1(S)-methylpropyl)- 2(S)-(3,3-dimethylbutyryloxy)-2- methyl-3-phenyl-propanamide | 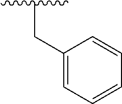 | CH₃ | 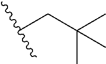 | 545 | 4.38 |
| 130 | N-(3-(4-chlorophenyl)-2(S)-(3- cyanophenyl)-1(S)-methylpropyl)- 2(R)-(3,3-dimethylbutyryloxy)-2- methyl-3-phenyl-propanamide | CH₃ | 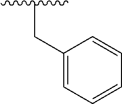 | 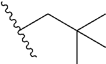 | 545 | 4.40 |
| 131 | N-(3-(4-chlorophenyl)-2(S)-(3- cyanophenyl)-1(S)-methylpropyl)- 2(S)-(cyclobutane-carbonyloxy)- 2-methyl-3-phenyl-propanamide | 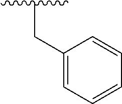 | CH₃ | 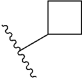 | 529 | 4.23 |
| 132 | N-(3-(4-chlorophenyl)-2(S)-(3- cyanophenyl)-1(S)-methylpropyl)- 2(R)-(cyclobutane-carbonyloxy)- 2-methyl-3-phenyl-propanamide | CH₃ | 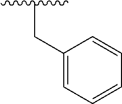 | 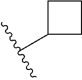 | 529 | 4.23 |

### Biological Example 1

### Cannabinoid Receptor-1 (CB1) Binding Assay

Binding affinity determination is based on recombinant human CB1 receptor expressed in Chinese Hamster Ovary (CHO) cells (Felder et al, Mol. Pharmacol. 48: 443-450, 1995). Total assay volume is 250 μL (240 μL CB1 receptor membrane solution plus 5 μL test compound solution plus 5 μL [3H]CP-55940 solution). Final concentration of [3H]CP-55940 is 0.6 nM. Binding buffer contains 50 mM Tris-HCl, pH7.4, 2.5 mM EDTA, 5 mM MgCl₂, 0.5 mg/mL fatty acid free bovine serum albumin and protease inhibitors (Cat#P8340, from Sigma). To initiate the binding reaction, 5 μL of radioligand solution is added, the mixture is incubated with gentle shaking on a shaker for 1.5 hours at 30° C. The binding is terminated by using 96-well harvester and filtering through GF/C filter presoaked in 0.05% polyethylenimine. The bound radiolabel is quantitated using scintillation counter. Apparent binding affinities for various compounds are calculated from IC₅₀ values (DeBlasi et al., Trends Pharmacol Sci 10: 227-229, 1989).

The binding assay for CB2 receptor is done similarly with recombinant human CB2 receptor expressed in CHO cells.

Selective CB1 antagonist/inverse agonist compounds have IC₅₀s 100-fold greater in the CB2 binding assay than in the CB1 assay, and generally have IC₅₀s of greater than one micromolar in the CB2 binding assay.

The compounds found in Examples 1 to 132 were tested in the above assay and found to have an IC₅₀ value of 100 nanomolar or less in the CB1 binding assay and 100 nanomolar or greater in the CB2 binding assay. In particular, the compound of Example 30 had an IC₅₀ value of 2.54 nM in the CB1 binding assay and 811 nM in the CB2 binding assay.

### Biological Example 2

### Cannabinoid Receptor-1 (CB1) Functional Activity Assay

The functional activation of CB1 receptor is based on recombinant human CB1 receptor expressed in CHO cells (Felder et al, Mol. Pharmacol. 48: 443-450, 1995). To determine the agonist activity or inverse agonist activity of any test compound, 50 μL of CB1-CHO cell suspension are mixed with test compound and 70 uL assay buffer containing 0.34 mM 3-isobutyl-1-methylxanthine and 5.1 μM of forskolin in 96-well plates. The assay buffer is comprised of Earle's Balanced Salt Solution supplemented with 5 mM MgCl₂, 1 mM glutamine, 10 mM HEPES, and 1 mg/mL bovine serum albumin. The mixture is incubated at room temperature for 30 minutes, and terminated by adding 30 μl/well of 0.5M HCl. The total intracellular cAMP level is quantitated using the New England Nuclear Flashplate and cAMP radioimmunoassay kit.

To determine the antagonist activity of test compound, the reaction mixture also contains 0.5 nM of the agonist CP55940, and the reversal of the CP55940 effect is quantitated. Alternatively, a series of dose response curves for CP55940 is performed with increasing concentration of the test compound in each of the dose response curves.

The functional assay for the CB2 receptor is done similarly with recombinant human CB2 receptor expressed in CHO cells.

CB1 antagonist/inverse agonist compounds of the present invention have EC₅₀s of less than 1 micromolar in the CB1 functional assay and selective CB1 antagonist/inverse agonists have generally have EC₅₀s of greater than 1 micromolar in the CB2 functional assay.

The compounds found in Examples 1 to 132 were tested in the above assay and found to have an EC₅₀ value of 100 nanomolar or less in the CB1 functional assay. In particular, the compound of Example 30 had an EC₅₀ value of 1.85 nM (−157% max) in the CB1 functional assay and 162 nM (−192% max) in the CB2 functional assay.

### Biological Example 3

### Acute Food Intake Studies in Rats or Mice: General Procedure

Adult rats or mice are used in these studies. After at least 2 days of acclimation to the vivarium conditions (controlled humidity and temperature, lights on for 12 hours out of 24 hours) food is removed from rodent cages. Experimental compounds or their vehicles are administered orally, intraperitoneally, subcutaneously or intravenously before the return of a known amount of food to cage. The optimal interval between dosing and food presentation is based on the half-life of the compound based on when brain concentrations of the compound is the highest. Food remaining is measured at several intervals. Food intake is calculated as grams of food eaten per gram of body weight within each time interval and the appetite-suppressant effect of the compounds are compared to the effect of vehicle. In these experiments many strains of mouse or rat, and several standard rodent chows can be used.

### Biological Example 4

### Chronic Weight Reduction Studies in Rats or Mice: General Procedure

Adult rats or mice are used in these studies. Upon or soon after weaning, rats or mice are made obese due to exclusive access to diets containing fat and sucrose in higher proportions than in the control diet. The rat strains commonly used include the Sprague Dawley bred through Charles River Laboratories. Although several mouse strains may be used, c57B1/6 mice are more prone to obesity and hyperinsulinemia than other strains. Common diets used to induce obesity include: Research Diets D12266B (32% fat) or D12451 (45% fat) and BioSery S3282 (60% fat). The rodents ingest chow until they are significantly heavier and have a higher proportion of body fat than control diet rats, often 9 weeks. The rodents receive injections (1 to 4 per day) or continuous infusions of experimental compounds or their vehicles either orally, intraperitoneally, subcutaneously or intravenously. Food intake and body weights are measured daily or more frequently. Food intake is calculated as grams of food eaten per gram of body weight within each time interval and the appetite-suppressant and weight loss effects of the compounds are compared to the effects of vehicle.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound of structural formula I: 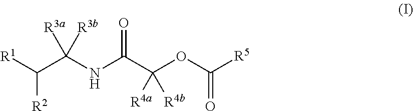
or a pharmaceutically acceptable salt thereof, wherein:
R¹ is selected from:
(1) C₁₋₁₀alkyl,
(2) C₃₋₁₀cycloalkyl,
(3) C₃₋₁₀cycloalkyl-C₁₋₄alkyl,
(4) cycloheteroalkyl,
(5) cycloheteroalkyl-C₁₋₄alkyl,
(6) aryl,
(7) aryl-C₁₋₄alkyl,
(8) heteroaryl,
(9) heteroaryl-C₁₋₄alkyl,
(10) —OR^{e},
(11) —NR^{c}R^{d},
(12) —NR^{c}C(O)R^{e},
(13) —CO₂R^{e}, and
(14) —C(O)NR^{c}R^{d},
wherein each alkyl is optionally substituted with one to four substituents independently selected from R^{a}, and cycloalkyl and cycloheteroalkyl are optionally substituted with one to four substituents selected from R^{b} and oxo, and aryl and heteroaryl are optionally substituted with one to four substituents independently selected from R^{b};
R² is selected from:
(1) C₁₋₁₀alkyl,
(2) C₃₋₁₀cycloalkyl-C₁₋₄alkyl,
(3) cycloheteroalkyl,
(4) cycloheteroalkyl-C₁₋₄alkyl,
(5) aryl,
(6) aryl-C₁₋₄alkyl,
(7) aryloxy,
(8) arylthio,
(9) heteroaryl, and
(10) heteroaryl-C₁₋₄alkyl,
wherein each alkyl is optionally substituted with one to four substituents independently selected from R^{a}, and cycloalkyl and cycloheteroalkyl are optionally substituted with one to four substituents selected from R^{b} and oxo, and aryl and heteroaryl are optionally substituted with one to four substituents independently selected from R^{b};
R^{3a} and R^{3b} are each independently selected from:
(1) hydrogen,
(2) aryl, and
(3) C₁₋₄alkyl,
wherein each alkyl is optionally substituted with one to four substituents independently selected from R^{a};
R^{4a} and R^{4b} are each independently selected from:
(1) hydrogen,
(2) C₁₋₈alkyl,
(3) C₃₋₈cycloalkyl,
(4) C₃₋₈cycloalkyl-C₁₋₄alkyl,
(5) cycloheteroalkyl,
(6) cycloheteroalkyl-C₁₋₄alkyl,
(7) aryl,
(8) aryl-C₁₋₄alkyl,
(9) heteroaryl, and
(10) heteroaryl-C₁₋₄alkyl, or
R^{4a} and R^{4b} together with the carbon to which they are attached form a carbocylic ring of 3 to 7 members containing 0-2 additional heteroatoms independently selected from oxygen, sulfur and N—Rg, wherein each alkyl is optionally substituted with one to four substituents independently selected from R^{a}, and cycloalkyl and cycloheteroalkyl are optionally substituted with one to four substituents selected from R^{b} and oxo, and aryl and heteroaryl are optionally substituted with one to four substituents independently selected from R^{b},
provided that R^{4a} and R^{4b} are not both hydrogen;
R⁵ is selected from:
(1) C₁₋₁₀alkyl,
(2) C₂₋₁₀alkenyl,
(3) C₂₋₁₀alkynyl,
(4) C₃₋₁₀cycloalkyl,
(5) C₃₋₁₀cycloalkyl-C₁₋₄alkyl,
(6) cycloheteroalkyl,
(7) cycloheteroalkyl-C₁₋₄alkyl,
(8) aryl,
(9) aryl-C₁₋₄ alkyl,
(10) heteroaryl,
(11) heteroaryl-C₁₋₄alkyl,
and
(12) —NR^{c}R^{d},
wherein alkyl, alkenyl, cycloalkyl, and cycloheteroalkyl are optionally substituted with one to four substituents independently selected from R^{a}, and cycloalkyl and cycloheteroalkyl are optionally substituted with one to four substituents selected from R^{b} and oxo, and aryl and heteroaryl are optionally substituted with one to four substituents independently selected from R^{b};
each R^{a} is independently selected from:
(1) —OR^{e},
(2) —NR^{c}S(O)ₘR^{e},
(3) halogen,
(4) —SR^{e},
(5) —S(O)ₘNR^{c}R^{d},
(6) —NR^{c}R^{d},
(7) —C(O)R^{e},
(8) —OC(O)R^{e},
(9) —CO₂R^{e},
(10) —CN,
(11) —C(O)NR^{c}R^{d},
(12) —NR^{c}C(O)R^{e},
(13) —NR^{c}C(O)OR^{e},
(14) —NR^{c}C(O)NR^{c}R^{d},
(15) —CF₃, and
(16) —OCF₃;
each R^{b} is independently selected from:
(1) R^{a},
(2) C₁₋₁₀alkyl,
(3) C₂₋₁₀ alkenyl,
(4) cycloalkyl,
(5) cycloalkyl-C₁₋₁₀alkyl,
(6) cycloheteroalkyl,
(7) cycloheteroalkyl-C₁₋₁₀ alkyl,
(8) aryl,
(9) heteroaryl,
(10) aryl-C₁₋₁₀alkyl, and
(11) heteroaryl-C₁₋₁₀alkyl,
wherein alkyl and alkenyl moieties are unsubstituted or substituted with one, two, three or four R^{k} substituents, and cycloalkyl, cycloheteroalkyl, aryl and heteroaryl moieties are unsubstituted or substituted with one, two or three R^{k} substituents;
R^{c} and R^{d} are independently selected from:
(1) hydrogen,
(2) C₁₋₁₀alkyl,
(3) C₂₋₁₀ alkenyl,
(4) cycloalkyl,
(5) cycloalkyl-C₁₋₁₀alkyl,
(6) cycloheteroalkyl,
(7) cycloheteroalkyl-C₁₋₁₀ alkyl,
(8) aryl,
(9) heteroaryl,
(10) aryl-C₁₋₁₀alkyl, and
(11) heteroaryl-C₁₋₁₀alkyl, or
R^{c} and R^{d} together with the atom(s) to which they are attached form a heterocyclic ring of 4 to 7 members containing 0-2 additional heteroatoms independently selected from oxygen, sulfur and N—Rg, wherein when R^{c} is not hydrogen, each R^{c} may be optionally or substituted with one to three substituents selected from R^{f}, and wherein when R^{d} is not hydrogen, each R^{d} may be optionally substituted with one to three substituents selected from R^{f};
each R^{e} is independently selected from:
(1) hydrogen,
(2) C₁₋₁₀ alkyl,
(3) C₂₋₁₀alkenyl,
(4) cycloalkyl,
(5) cycloalkyl-C₁₋₁₀alkyl,
(6) cycloheteroalkyl,
(7) cycloheteroalkyl-C₁₋₁₀ alkyl,
(8) aryl,
(9) heteroaryl,
(10) aryl-C₁₋₁₀alkyl, and
(11) heteroaryl-C₁₋₁₀alkyl,
wherein, when R^{e} is other than hydrogen, each R^{e} may be unsubstituted or substituted with one to three substituents selected from R^{f};
each R^{f} is independently selected from:
(1) halogen,
(2) C₁₋₁₀alkyl,
(3) C₂₋₁₀alkenyl,
(4) —CN,
(5) —CF₃,
(6) —OCF₃,
(7) —OR^{b},
(8) —NHS(O)ₘR^{h},
(9) —SR^{h},
(10) —S(O)ₘNR^{h}R^{h},
(11) —NR^{h}R^{h},
(12) —C(O)R^{h},
(13) —CO₂R^{h},
(14) —C(O)NR^{h}R^{h},
(15) —NR^{h}C(O)R^{h},
(16) —NR^{h}C(O)OR^{h},
(17) —NR^{h}C(O)NR^{h}R^{h},
(18) cycloheteroalkyl,
(19) cycloalkyl,
(20) cycloalkyl-C₁₋₁₀ alkyl,
(21) cycloheteroalkyl,
(22) cycloheteroalkyl-C₁₋₁₀ alkyl,
(23) aryl,
(24) heteroaryl,
(25) aryl-C₁₋₁₀alkyl, and
(26) heteroaryl-C₁₋₁₀alkyl;
each Rg is independently selected from
(1) C₁₋₁₀alkyl, and
(2) —C(O)R^{c};
each R^{h} is independently selected from:
(1) hydrogen,
(2) C₁₋₁₀ alkyl,
(3) C₂₋₁₀ alkenyl,
(4) cycloalkyl,
(5) cycloalkyl-C₁₋₁₀alkyl,
(6) cycloheteroalkyl,
(7) cycloheteroalkyl-C₁₋₁₀ alkyl,
(8) aryl,
(9) heteroaryl,
(10) aryl-C₁₋₁₀alkyl, and
(11) heteroaryl-C₁₋₁₀alkyl,
wherein when R^{h} is not hydrogen, each R^{h} may be unsubstituted or substituted with one, two or three substituents selected from Rⁱ;
each Rⁱ is independently selected from:
(1) halogen,
(2) C₁₋₁₀alkyl,
(3) —O—C₁₋₄alkyl,
(4) —OH,
(5) —S—C₁₋₄alkyl,
(6) —CN,
(7) —CF₃, and
(8) —OCF₃;
each R^{k} is independently selected from:
(1) halogen,
(2) C₁₋₄alkyl,
(3) —O—C₁₋₄alkyl,
(4) —S—C₁₋₄alkyl,
(5) —CN,
(6) —CF₃, and
(7) —OCF₃; and
m is selected from 1 and 2;
provided that, when R² is unsubstituted alkyl and R⁵ is unsubstituted alkyl, then R¹ is not unsubstituted.

2. The compound according to claim 1, wherein:
R¹ is selected from: cyclobutyl, cyclopentyl, cyclohexyl, pyrrolidinyl, phenyl, pyridyl, indolinyl, benzisoxazolyl, azaindolyl, 2,3-dihydroindolyl, 3,4-dihydroquinolinyl, pyridazinyl, pyrimidinyl, pyrrolidinyl, triazolyl, benzotriazolyl, thienyl, indolyl, indazolyl, —OR^{e}, —NR^{c}R^{d}, and —C(O)OR^{e}, wherein each cycloalkyl and cycloheteroalkyl moiety is optionally substituted with one to three substituents selected from R^{b} and oxo, and each aryl and heteroaryl moiety is optionally substituted with one to four substituents independently selected from R^{b};
R² is selected from: C₃₋₅alkyl, C₃₋₆cycloalkyl-methyl-, piperidinyl-methyl, phenyl, benzyl, phenyloxy, phenylthio, pyridyl, and pyridyl-methyl, wherein each piperidinyl, phenyl and pyridyl is unsubstituted or substituted with one or two substituents independently selected from: halo-, methoxy, methoxycarbonyl, cyano, methyl, and t-butyloxycarbonyl;
R^{3a} is methyl;
R^{3b} is selected from: hydrogen, and methyl;
R^{4a} and R^{4b} are each independently selected from: hydrogen, methyl, ethyl, isopropyl, t-butyl, phenyl, and phenyl-methyl-, or R^{4a} and R^{4b} together with the carbon to which they are attached form a cyclopropyl ring, provided that R^{4a} and R^{4b} are not both hydrogen;
R⁵ is selected from: methyl, ethyl, isopropyl, 2,2-dimethylpropyl, t-butyl, cyclobutyl, cyclopentyl, cyclohexyl, tetrahydrofuranyl, phenyl, phenyl-methyl-, pyridyl, pyridylmethyl, thiadiazolyl, pyrazolyl, pyrazinyl, isoxazolyl, thiazolyl, pyrimidinyl, oxazolyl, oxadiazolyl, triazolyl, imidazolyl, pyrazolo[1,5-a]pyrimidinyl, imidazo[2,1-b]thiazolyl, imidazo[1,2-a]pyridyl, and anilino, wherein alkyl moieties are optionally substituted with one to three substituents independently selected from R^{a}, and cycloalkyl and cycloheteroalkyl are optionally substituted with one to three substituents selected from R^{b} and oxo, and each aryl and heteroaryl are optionally substituted with one to three substituents independently selected from R^{b};
each R^{a} is independently selected from: —OR^{e}, —NHS(O)₂R^{e}, halogen, —SR^{e}, —S(O)₂NR^{c}R^{d}, —NR^{c}R^{d}, —C(O)R^{e}, —OC(O)R^{e}, —CO₂R^{e}, —CN, —C(O)NR^{c}R^{d}, —NHC(O)R^{e}, —NHC(O)OR^{e}, —NHC(O)NR^{c}R^{d}, —CF₃, and —OCF₃;
each R^{b} is independently selected from: —OR^{e}, halogen, —SCH₃, —NR^{c}R^{d}, —C(O)CH₃, —OC(O)R^{e}, —CO₂R^{e}, —CN, —C(O)NR^{c}R^{d}, —NHC(O)R^{e}, —NHC(O)OR^{e}, —CF₃, —OCF₃, C₁₋₆alkyl, cycloalkyl, cycloheteroalkyl, phenyl, and heteroaryl, wherein alkyl is are unsubstituted or substituted with one, two, three or four R^{k} substituents, and cycloalkyl, cycloheteroalkyl, aryl and heteroaryl moieties are unsubstituted or substituted with one, two or three R^{k} substituents;
each R^{c} is independently selected from: hydrogen, C₁₋₆alkyl, cycloalkyl, cycloheteroalkyl, phenyl, and heteroaryl, wherein when R^{c} is not hydrogen, each R^{c} may be optionally substituted with one to three substituents selected from R^{f};
each R^{d} is independently selected from: hydrogen, C₁₋₆alkyl, cycloalkyl, cycloheteroalkyl, aryl, and heteroaryl, wherein, when R^{d} is not hydrogen, each R^{d} may be optionally substituted with one to three substituents selected from R^{f};
or R^{c} and R^{d} together with the atom(s) to which they are attached form a heterocyclic ring of 4 to 7 members, wherein the heterocyclic ring formed by R^{c} and R^{d} may be unsubstituted or substituted with one to three substituents selected from R^{f};
each R^{e} is independently selected from: hydrogen, C₁₋₆alkyl, cycloalkyl, cycloheteroalkyl, aryl, and heteroaryl, wherein, when R^{e} is other than hydrogen, each R^{e} may be unsubstituted or substituted with one to three substituents selected from R^{f};
each R^{f} is independently selected from: —F, —Cl, —I, methyl, ethyl, isopropyl, t-butyl, —S—CH₃, —CN, —CF₃, —OCF₃, —OCH₃, —NH₂, —NHCH₃, —N(CH₃)₂, —C(O)CH₃, —CO₂CH₃, —CO₂C(CH₃)₃, 2H-tetrazolyl, cyclopropyl, phenyl, thiazolyl, and pyridyl;
each Rg is methyl or methylcarbonyl;
each R^{h} is independently selected from: hydrogen, and methyl;
each Rⁱ is independently selected from: —F, —Cl, —CH₃, —O—CH₃, —S—CH₃, —CN, —CF₃, and —OCF₃; and
each R^{k} is independently selected from: —F, —Cl, and —CH₃;
or a pharmaceutically acceptable salt thereof.

3. The compound according to claim 2 of structural formula IC: 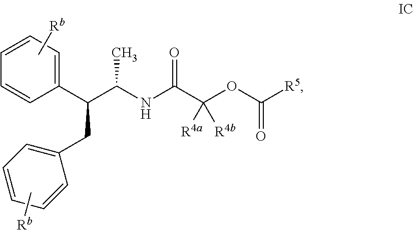
or a pharmaceutically acceptable salt thereof, wherein:
R^{b}, R^{4a}, R^{4b} and R⁵ are as in claim 2.

4. The compound according to claim 2 of structural formula ID: 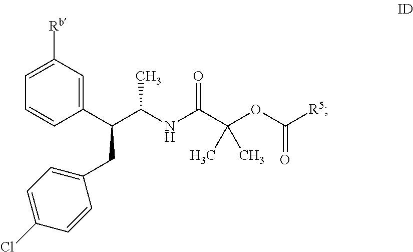
or a pharmaceutically acceptable salt thereof, wherein:
Rb′ is selected from —CN, and 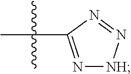
R⁵ is selected from: methyl, ethyl, isopropyl, 2,2-dimethylpropyl, t-butyl, cyclobutyl, cyclopentyl, cyclohexyl, tetrahydrofuranyl, phenyl, phenyl-methyl-, pyridyl, pyridylmethyl-, thiadiazolyl, pyrazolyl, pyrazinyl, isoxazolyl, thiazolyl, pyrimidinyl, oxazolyl, oxadiazolyl, triazolyl, imidazolyl, pyrazolo[1,5-a]pyrimidinyl, imidazo[2,1-b]thiazolyl, imidazo[1,2-a]pyridyl, and anilino, wherein alkyl moieties are optionally substituted with one to three substituents independently selected from R^{a}, and cycloalkyl and cycloheteroalkyl are optionally substituted with one to three substituents selected from R^{b} and oxo, and each aryl and heteroaryl are optionally substituted with one to three substituents independently selected from R^{b};
each R^{a} is independently selected from: hydroxy, methoxy, methylcarbonyloxy, fluoro, chloro, methylthio, amino, N,N-dimethylamino, N-methylamino, methylcarbonyl, methoxycarbonyl, —CN, N-methylcarbonyl-amino-, N-(t-butyloxycarbonyl)amino-, —CF₃, and —OCF₃; and
each R^{b} is independently selected from:
(1) —OH,
(2) —OCH₃,
(3) —OCH₂CF₃,
(4) —Cl
(5) —F,
(6) —Br,
(7) —I,
(8) —SCH₃,
(9) —NH₂,
(10) —OC(O)CH₃,
(11) t-butyloxycarbonyl-,
(12) —CN,
(13) —NHC(O)CF₃,
(14) —CF₃,
(15) —OCF₃,
(16) methyl,
(17) ethyl,
(18) isopropyl,
(19) t-butyl,
(20) —CF₂—CF₂H,
(21) cyclopropyl,
(22) 2-H-tetrazolyl,
(23) cycloheteroalkyl,
(24) phenyl, unsubstituted or substituted with a methyl or halogen substituent, and
(25) 1,2,3-thiazolyl unsubstituted or substituted with a methyl or halogen substituent.

5. The compound according to claim 1, selected from:
(1) N-(3-(4-chlorophenyl)-2(S)-(3-cyanophenyl)-1(S)-methylpropyl)-2-benzoyloxy-2-methyl-propanamide,
(2) N-(3-(4-chlorophenyl)-2(S)-(3-cyanophenyl)-1(S)-methylpropyl)-2-acetoxy-2-methyl-propanamide,
(3) N-(3-(4-chlorophenyl)-2(S)-(3-cyanophenyl)-1(S)-methylpropyl)-2-nicotinoyloxy-2-methyl-propanamide,
(4) N-(3-(4-chlorophenyl)-2(S)-(3-cyanophenyl)-1(S)-methylpropyl)-2-picolinoyloxy-2-methyl-propanamide,
(5) N-(3-(4-chlorophenyl)-2(S)-(3-cyanophenyl)-1(S)-methylpropyl)-2-isonicotinoyloxy-2-methyl-propanamide,
(6) N-(3-(4-chlorophenyl)-2(S)-(3-cyanophenyl)-1(S)-methylpropyl)-2-isobutanoyloxy-2-methyl-propanamide,
(7) N-(3-(4-chlorophenyl)-2(S)-(3-cyanophenyl)-1(S)-methylpropyl)-2-cyclohexylcarbonyloxy-2-methyl-propanamide,
(8) N-(3-(4-chlorophenyl)-2(S)-(3-cyanophenyl)-1(S)-methylpropyl)-2-cyclohexylcarbonyloxy-2-methyl-propanamide,
(9) N-(3-(4-chlorophenyl)-2(S)-(3-cyanophenyl)-1(S)-methylpropyl)-2-cyclopentylcarbonyloxy-2-methyl-propanamide,
(10) N-(3-(4-chlorophenyl)-2(S)-(3-cyanophenyl)-1(S)-methylpropyl)-2-(4-cyano-benzoyloxy)-2-methyl-propanamide,
(11) N-(3-(4-chlorophenyl)-2(S)-(3-cyanophenyl)-1(S)-methylpropyl)-2-(2-trifluoromethoxy-benzoyloxy)-2-methyl-propanamide,
(12) N-(3-(4-chlorophenyl)-2(S)-(3-cyanophenyl)-1(S)-methylpropyl)-2-(2-chloro-benzoyloxy)-2-methyl-propanamide,
(13) N-(3-(4-chlorophenyl)-2(S)-(3-cyanophenyl)-1(S)-methylpropyl)-2-(4-isopropyl-1,2,3-thiadiazolyl-5-carbonyloxy)-2-methyl-propanamide,
(14) N-(3-(4-chlorophenyl)-2(S)-(3-cyanophenyl)-1(S)-methylpropyl)-2-(2-hydroxy-benzoyloxy)-2-methyl-propanamide,
(15) N-(3-(4-chlorophenyl)-2(S)-(3-cyanophenyl)-1(S)-methylpropyl)-2-(5-trifluoromethyl-1-phenyl-pyrazolyl-4-carbonyloxy)-2-methyl-propanamide,
(16) N-(3-(4-chlorophenyl)-2(S)-(3-cyanophenyl)-1(S)-methylpropyl)-2-(2-fluoro-benzoyloxy)-2-methyl-propanamide,
(17) N-(3-(4-chlorophenyl)-2(S)-(3-cyanophenyl)-1(S)-methylpropyl)-2-(1,3-dimethyl-pyrazolyl-5-carbonyloxy)-2-methyl-propanamide,
(18) N-(3-(4-chlorophenyl)-2(S)-(3-cyanophenyl)-1(S)-methylpropyl)-2-pyrazinylcarbonyloxy-2-methyl-propanamide,
(19) N-(3-(4-chlorophenyl)-2(S)-(3-cyanophenyl)-1(S)-methylpropyl)-2-(3-phenyl-isoxazolyl-5-carbonyloxy)-2-methyl-propanamide,
(20) N-(3-(4-chlorophenyl)-2(S)-(3-cyanophenyl)-1(S)-methylpropyl)-2-(pyrazolo[1,5-a]pyrimidinyl-3-carbonyloxy)-2-methyl-propanamide,
(21) N-(3-(4-chlorophenyl)-2(S)-(3-cyanophenyl)-1(S)-methylpropyl)-2-((2R)-(acetoxy)(phenyl)acetoxy)-2-methyl-propanamide,
(22) N-(3-(4-chlorophenyl)-2(S)-(3-cyanophenyl)-1(S)-methylpropyl)-2-(2-acetoxy-acetoxy)-2-methyl-propanamide,
(23) N-(3-(4-chlorophenyl)-2(S)-(3-cyanophenyl)-1(S)-methylpropyl)-2-((25)-(hydroxy)(phenyl)acetoxy)-2-methyl-propanamide,
(24) N-(3-(4-chlorophenyl)-2(S)-(3-cyanophenyl)-1(S)-methylpropyl)-2-(5-methyl-pyrazinyl)carbonyloxy)-2-methyl-propanamide,
(25) N-(3-(4-chlorophenyl)-2(S)-(3-cyanophenyl)-1(S)-methylpropyl)-2-(4-phenyl-1,2,3-thiadiazolyl-5-carbonyloxy)-2-methyl-propanamide,
(26) N-(3-(4-chlorophenyl)-2(S)-(3-cyanophenyl)-1(S)-methylpropyl)-2-(isoxazolyl-5-carbonyloxy)-2-methyl-propanamide,
(27) N-(3-(4-chlorophenyl)-2(S)-(3-cyanophenyl)-1(S)-methylpropyl)-2-(3-tert-butyl-1-methyl-pyrazolyl-5-carbonyloxy)-2-methyl-propanamide,
(28) N-(3-(4-chlorophenyl)-2(S)-(3-cyanophenyl)-1(S)-methylpropyl)-2-(1-tert-butyl-3-methyl-pyrazolyl-5-carbonyloxy)-2-methyl-propanamide,
(29) N-(3-(4-chlorophenyl)-2(S)-(3-cyanophenyl)-1(S)-methylpropyl)-2-(5-methyl-isoxazolyl-3-carbonyloxy)-2-methyl-propanamide,
(30) N-(3-(4-chlorophenyl)-2(S)-(3-cyanophenyl)-1(S)-methylpropyl)-2-(2,6-dichloro-benzoyloxy)-2-methyl-propanamide,
(31) N-(3-(4-chlorophenyl)-2(S)-(3-cyanophenyl)-1(S)-methylpropyl)-2-(2,6-difluoro-benzoyloxy)-2-methyl-propanamide,
(32) N-(3-(4-chlorophenyl)-2(S)-(3-cyanophenyl)-1(S)-methylpropyl)-2-(4-acetoxy-benzoyloxy)-2-methyl-propanamide,
(33) N-(3-(4-chlorophenyl)-2(S)-(3-cyanophenyl)-1(S)-methylpropyl)-2-(3,5-dimethyl-isoxazolyl-4-carbonyloxy)-2-methyl-propanamide,
(34) N-(3-(4-chlorophenyl)-2(S)-(3-cyanophenyl)-1(S)-methylpropyl)-2-(1-methyl-5-trifluoromethyl-pyrazolyl-3-carbonyloxy)-2-methyl-propanamide,
(35) N-(3-(4-chlorophenyl)-2(S)-(3-cyanophenyl)-1(S)-methylpropyl)-2-(4-hydroxy-benzoyloxy)-2-methyl-propanamide,
(36) N-(3-(4-chlorophenyl)-2(S)-(3-cyanophenyl)-1(S)-methylpropyl)-2-(1,5-dimethyl-pyrazolyl-4-carbonyloxy)-2-methyl-propanamide,
(37) N-(3-(4-chlorophenyl)-2(S)-(3-cyanophenyl)-1(S)-methylpropyl)-2-((2S)-(acetoxy)(phenyl)acetoxy)-2-methyl-propanamide,
(38) N-(3-(4-chlorophenyl)-2(S)-(3-cyanophenyl)-1(S)-methylpropyl)-2-((2R)-(hydroxy)(phenyl)acetoxy)-2-methyl-propanamide,
(39) N-(3-(4-chlorophenyl)-2(S)-(3-cyanophenyl)-1(S)-methylpropyl)-2-(3-acetoxy-benzoyloxy)-2-methyl-propanamide,
(40) N-(3-(4-chlorophenyl)-2(S)-(3-cyanophenyl)-1(S)-methylpropyl)-2-(3-hydroxy-benzoyloxy)-2-methyl-propanamide,
(41) N-(3-(4-chlorophenyl)-2(S)-(3-cyanophenyl)-1(S)-methylpropyl)-2-(2,4-dimethyl-thiazolyl-5-carbonyloxy)-2-methyl-propanamide,
(42) N-(3-(4-chlorophenyl)-2(S)-(3-cyanophenyl)-1(S)-methylpropyl)-2-(1-methyl-3-trifluoromethyl-pyrazolyl-5-carbonyloxy)-2-methyl-propanamide,
(43) N-(3-(4-chlorophenyl)-2(S)-(3-cyanophenyl)-1(S)-methylpropyl)-2-(pyrimidinyl-5-carbonyloxy)-2-methyl-propanamide,
(44) N-(3-(4-chlorophenyl)-2(S)-(3-cyanophenyl)-1(S)-methylpropyl)-2-(2-methyl-5-trifluoromethyl-oxazolyl-3-carbonyloxy)-2-methyl-propanamide,
(45) N-(3-(4-chlorophenyl)-2(S)-(3-cyanophenyl)-1(S)-methylpropyl)-2-phenylacetoxy-2-methyl-propanamide,
(46) N-(3-(4-chlorophenyl)-2(S)-(3-cyanophenyl)-1(S)-methylpropyl)-2-((2R)-(tert-butyloxycarbonylamino)-(phenyl)acetoxy)-2-methyl-propanamide,
(47) N-(3-(4-chlorophenyl)-2(S)-(3-cyanophenyl)-1(S)-methylpropyl)-2-((2S)-(tert-butyloxycarbonylamino)-(phenyl)acetoxy)-2-methyl-propanamide,
(48) N-(3-(4-chlorophenyl)-2(S)-(3-cyanophenyl)-1(S)-methylpropyl)-2-(5-methyl-3-phenyl-isoxazolyl-4-carbonyloxy)-2-methyl-propanamide,
(49) N-(3-(4-chlorophenyl)-2(S)-(3-cyanophenyl)-1(S)-methylpropyl)-2-(1-(4-chlorophenyl)-5-trifluoromethyl-pyrazolyl-4-carbonyloxy)-2-methyl-propanamide,
(50) N-(3-(4-chlorophenyl)-2(S)-(3-cyanophenyl)-1(S)-methylpropyl)-2-(1-ethyl-3-methyl-pyrazolyl-5-carbonyloxy)-2-methyl-propanamide,
(51) N-(3-(4-chlorophenyl)-2(S)-(3-cyanophenyl)-1(S)-methylpropyl)-2-(1,3,5-trimethyl-pyrazolyl-4-carbonyloxy)-2-methyl-propanamide,
(52) N-(3-(4-chlorophenyl)-2(S)-(3-cyanophenyl)-1(S)-methylpropyl)-2-(6-methyl-imidazo[2,1-b]thiazolyl-5-carbonyloxy)-2-methyl-propanamide,
(53) N-(3-(4-chlorophenyl)-2(S)-(3-cyanophenyl)-1(S)-methylpropyl)-2-(2-(2-pyridyl)acetoxy)-2-methyl-propanamide,
(54) N-(3-(4-chlorophenyl)-2(S)-(3-cyanophenyl)-1(S)-methylpropyl)-2-(1-phenyl-pyrazolyl-4-carbonyloxy)-2-methyl-propanamide,
(55) N-(3-(4-chlorophenyl)-2(S)-(3-cyanophenyl)-1(S)-methylpropyl)-2-(2-methyl-imidazo[1,2-a]pyridyl 4-carbonyloxy)-2-methyl-propanamide,
(56) N-(3-(4-chlorophenyl)-2(S)-(3-cyanophenyl)-1(S)-methylpropyl)-2-(2-phenyl-5-trifluoromethyl-oxazolyl-4-carbonyloxy)-2-methyl-propanamide,
(57) N-(3-(4-chlorophenyl)-2(S)-(3-cyanophenyl)-1(S)-methylpropyl)-2-(1-methyl-imidazolyl-2-carbonyloxy)-2-methyl-propanamide,
(58) N-(3-(4-chlorophenyl)-2(S)-(3-cyanophenyl)-1(S)-methylpropyl)-2-(1-methyl-imidazolyl-5-carbonyloxy)-2-methyl-propanamide,
(59) N-(3-(4-chlorophenyl)-2(S)-(3-cyanophenyl)-1(S)-methylpropyl)-2-(pyrazolo[1,5-a]pyridyl-3-carbonyloxy)-2-methyl-propanamide,
(60) N-(3-(4-chlorophenyl)-2(S)-(3-cyanophenyl)-1(S)-methylpropyl)-2-(1-isopropyl-pyrazolyl-4-carbonyloxy)-2-methyl-propanamide,
(61) N-(3-(4-chlorophenyl)-2(S)-(3-cyanophenyl)-1(S)-methylpropyl)-2-(1-methyl-imidazolyl-4-carbonyloxy)-2-methyl-propanamide,
(62) N-(3-(4-chlorophenyl)-2(S)-(3-cyanophenyl)-1(S)-methylpropyl)-2-(4-methyl-1,2,5-oxadiazolyl-3-carbonyloxy)-2-methyl-propanamide,
(63) N-(3-(4-chlorophenyl)-2(S)-(3-cyanophenyl)-1(S)-methylpropyl)-2-(5-methyl-2-phenyl-2H-1,2,3-triazolyl-4-carbonyloxy)-2-methyl-propanamide,
(64) N-(3-(4-chlorophenyl)-2(S)-(3-cyanophenyl)-1(S)-methylpropyl)-2-(5-isopropyl-isoxazolyl-3-carbonyloxy)-2-methyl-propanamide,
(65) N-(3-(4-chlorophenyl)-2(S)-(3-cyanophenyl)-1(S)-methylpropyl)-2-(3-methyl-5-(4-methyl-1,2,3-thiazol-5-yl)isoxazolyl-4-carbonyloxy)-2-methyl-propanamide,
(66) N-(3-(4-chlorophenyl)-2(S)-(3-cyanophenyl)-1(S)-methylpropyl)-2-(1-methyl-pyrazolyl-4-carbonyloxy)-2-methyl-propanamide,
(67) N-(3-(4-chlorophenyl)-2(S)-(3-cyanophenyl)-1(S)-methylpropyl)-2-(5-cyclopropyl-isoxazolyl-3-carbonyloxy)-2-methyl-propanamide,
(68) N-(3-(4-chlorophenyl)-2(S)-(3-cyanophenyl)-1(S)-methylpropyl)-2-(3-methyl-5-phenyl-isoxazolyl-4-carbonyloxy)-2-methyl-propanamide,
(69) N-(3-(4-chlorophenyl)-2(S)-(3-cyanophenyl)-1(S)-methylpropyl)-2-(1-methyl-3-trifluoromethyl-1H-pyrazolyl-4-carbonyloxy)-2-methyl-propanamide,
(70) N-(3-(4-chlorophenyl)-2(S)-(3-cyanophenyl)-1(S)-methylpropyl)-2-(5-phenyl-1,2,4-oxadiazolyl-3-carbonyloxy)-2-methyl-propanamide,
(71) N-(3-(4-chlorophenyl)-2(S)-(3-cyanophenyl)-1(S)-methylpropyl)-2-(2-acetoxy-benzoyloxy)-2-methyl-propanamide,
(72) N-(3-(4-chlorophenyl)-2(S)-(3-cyanophenyl)-1(S)-methylpropyl)-2-(2-methyl-benzoyloxy)-2-methyl-propanamide,
(73) N-(3-(4-chlorophenyl)-2(S)-(3-cyanophenyl)-1(S)-methylpropyl)-2-(5-phenyl-isoxazolyl-3-carbonyloxy)-2-methyl-propanamide,
(74) N-(3-(4-chlorophenyl)-2(S)-(3-cyanophenyl)-1(S)-methylpropyl)-2-(pivaloyloxy)-2-methyl-propanamide,
(75) N-(3-(4-chlorophenyl)-2(S)-(3-cyanophenyl)-1(S)-methylpropyl)-2-(2-ethyl-benzoyloxy)-2-methyl-propanamide,
(76) N-(3-(4-chlorophenyl)-2(S)-(3-cyanophenyl)-1(S)-methylpropyl)-2-(2,6-dimethyl-benzoyloxy)-2-methyl-propanamide,
(77) N-(3-(4-chlorophenyl)-2(S)-(3-cyanophenyl)-1(S)-methylpropyl)-2-(2,4-dichloro-benzoyloxy)-2-methyl-propanamide,
(78) N-(3-(4-chlorophenyl)-2(S)-(3-cyanophenyl)-1(S)-methylpropyl)-2-(4-fluoro-2-methyl-benzoyloxy)-2-methyl-propanamide,
(79) N-(3-(4-chlorophenyl)-2(S)-(3-cyanophenyl)-1(S)-methylpropyl)-2-(4-fluoro-2-trifluoromethyl-benzoyloxy)-2-methyl-propanamide,
(80) N-(3-(4-chlorophenyl)-2(S)-(3-cyanophenyl)-1(S)-methylpropyl)-2-(2-trifluoromethyl-benzoyloxy)-2-methyl-propanamide,
(81) N-(3-(4-chlorophenyl)-2(S)-(3-cyanophenyl)-1(S)-methylpropyl)-2-(2-methoxy-benzoyloxy)-2-methyl-propanamide,
(82) N-(3-(4-chlorophenyl)-2(S)-(3-cyanophenyl)-1(S)-methylpropyl)-2-(2,6-dimethoxy-benzoyloxy)-2-methyl-propanamide,
(83) N-(3-(4-chlorophenyl)-2(S)-(3-cyanophenyl)-1(S)-methylpropyl)-2-(3-cyano-benzoyloxy)-2-methyl-propanamide,
(84) N-(3-(4-chlorophenyl)-2(S)-(3-cyanophenyl)-1(S)-methylpropyl)-2-(2-chloro-6-fluoro-benzoyloxy)-2-methyl-propanamide,
(85) N-(3-(4-chlorophenyl)-2(S)-(3-cyanophenyl)-1(S)-methylpropyl)-2-(4-trifluoromethoxy-benzoyloxy)-2-methyl-propanamide,
(86) N-(3-(4-chlorophenyl)-2(S)-(3-cyanophenyl)-1(S)-methylpropyl)-2-(tetrahydrofuranyl-2(S)-carbonyloxy)-2-methyl-propanamide,
(87) N-(3-(4-chlorophenyl)-2(S)-(3-cyanophenyl)-1(S)-methylpropyl)-2-(2-acetamido-6-fluoro-benzoyloxy)-2-methyl-propanamide,
(88) N-(3-(4-chlorophenyl)-2(S)-(3-cyanophenyl)-1(S)-methylpropyl)-2-(2-amino-benzoyloxy)-2-methyl-propanamide,
(89) N-(3-(4-chlorophenyl)-2(S)-(3-cyanophenyl)-1(S)-methylpropyl)-2-(5-oxo-tetrahydrofuranyl-2(R)-carbonyloxy)-2-methyl-propanamide,
(90) N-(3-(4-chlorophenyl)-2(S)-(3-cyanophenyl)-1(S)-methylpropyl)-2-(5-oxo-tetrahydrofuranyl-2(S)-carbonyloxy)-2-methyl-propanamide,
(91) N-(3-(4-chlorophenyl)-2(S)-(3-cyanophenyl)-1(S)-methylpropyl)-2-(4-chloro-benzoyloxy)-2-methyl-propanamide,
(92) N-(3-(4-chlorophenyl)-2(S)-(3-cyanophenyl)-1(S)-methylpropyl)-2-(6-chloro-2-fluoro-3-methyl-benzoyloxy)-2-methyl-propanamide,
(93) N-(3-(4-chlorophenyl)-2(S)-(3-cyanophenyl)-1(S)-methylpropyl)-2-(3,3-dimethyl-butyryloxy)-2-methyl-propanamide,
(94) N-(3-(4-chlorophenyl)-2(S)-(3-cyanophenyl)-1(S)-methylpropyl)-2-anilino-2-methyl-propanamide,
(95) N-(3-(4-chlorophenyl)-2(S)-(3-cyanophenyl)-1(S)-methylpropyl)-2-(3,4-dichloro-benzoyloxy)-2-methyl-propanamide,
(96) N-(3-(4-chlorophenyl)-2(S)-(3-cyanophenyl)-1(S)-methylpropyl)-2-(2-chloro-3,6-dichloro-benzoyloxy)-2-methyl-propanamide,
(97) N-(3-(4-chlorophenyl)-2(S)-(3-cyanophenyl)-1(S)-methylpropyl)-2-(2-chloro-6-fluoro-3-methyl-benzoyloxy)-2-methyl-propanamide,
(98) N-(3-(4-chlorophenyl)-2(S)-(3-cyanophenyl)-1(S)-methylpropyl)-2-(2-amino-6-chloro-benzoyloxy)-2-methyl-propanamide,
(99) N-(3-(4-chlorophenyl)-2(S)-(3-cyanophenyl)-1(S)-methylpropyl)-2-(2-chloro-6-methyl-benzoyloxy)-2-methyl-propanamide,
(100) N-(3-(4-chlorophenyl)-2(S)-(3-cyanophenyl)-1(S)-methylpropyl)-2-(2-phenyl-tetrahydrofuranyl-2-carbonyloxy)-2-methyl-propanamide,
(101) N-(3-(4-chlorophenyl)-2(S)-(3-cyanophenyl)-1(S)-methylpropyl)-2-(2-methyl-tetrahydrofuranyl-2-carbonyloxy)-2-methyl-propanamide,
(102) N-(3-(4-chlorophenyl)-2(S)-(3-cyanophenyl)-1(S)-methylpropyl)-2-(2-methyl-5-oxo-tetrahydrofuranyl-2-carbonyloxy)-2-methyl-propanamide,
(103) N-(3-(4-chlorophenyl)-2(S)-(3-cyanophenyl)-1(S)-methylpropyl)-2-(6-methyl-2-trifluoroacetamido-benzoyloxy)-2-methyl-propanamide,
(104) N-(3-(4-chlorophenyl)-2(S)-(3-cyanophenyl)-1(S)-methylpropyl)-2-(2-amino-6-fluoro-benzoyloxy)-2-methyl-propanamide,
(105) N-(3-(4-chlorophenyl)-2(S)-(3-cyanophenyl)-1(S)-methylpropyl)-2-(2-amino-nicotinoyloxy)-2-methyl-propanamide,
(106) N-(3-(4-chlorophenyl)-2(S)-(3-cyanophenyl)-1(S)-methylpropyl)-2-(2-fluoro-isonicotinoyloxy)-2-methyl-propanamide,
(107) N-(3-(4-chlorophenyl)-2(S)-(3-cyanophenyl)-1(S)-methylpropyl)-2-(2-(2,2,2-trifluoroethoxy)-benzoyloxy)-2-methyl-propanamide,
(108) N-(3-(4-chlorophenyl)-2(S)-(3-cyanophenyl)-1(S)-methylpropyl)-2-(2-(1,1,2,2-tetrafluoroethyl)-benzoyloxy)-2-methyl-propanamide,
(109) N-(3-(4-chlorophenyl)-2(S)-(3-cyanophenyl)-1(S)-methylpropyl)-2-(2-acetamido-6-chloro-benzoyloxy)-2-methyl-propanamide,
(110) N-(3-(4-chlorophenyl)-2(S)-(3-cyanophenyl)-1(S)-methylpropyl)-2-(2-chloro-6-methyl-isonicotinoyloxy)-2-methyl-propanamide,
(111) N-(3-(4-chlorophenyl)-2(S)-(3-cyanophenyl)-1(S)-methylpropyl)-2-(2-amino-6-methyl-benzoyloxy)-2-methyl-propanamide,
(112) N-(3-(4-chlorophenyl)-2-(3-(2H-tetrazol-5-yl)phenyl)-1-methylpropyl)-2-(4-(2H-tetrazol-5-yl)benzoyloxy)-2-methyl-propanamide,
(113) N-(3-(4-chlorophenyl)-2(S)-(3-cyanophenyl)-1(S)-methylpropyl)-2(S)-benzoyloxy)-propanamide,
(114) N-(3-(4-chlorophenyl)-2(S)-(3-cyanophenyl)-1(S)-methylpropyl)-2(R)-benzoyloxy)-propanamide,
(115) N-(3-(4-chlorophenyl)-2(S)-(3-cyanophenyl)-1(S)-methylpropyl)-2(S)-benzoyloxy)-butanamide,
(116) N-(3-(4-chlorophenyl)-2(S)-(3-cyanophenyl)-1(S)-methylpropyl)-2(R)-benzoyloxy)-butanamide,
(117) N-(3-(4-chlorophenyl)-2(S)-(3-cyanophenyl)-1(S)-methylpropyl)-1-benzoyloxy)-cyclopropane-1-carboxamide,
(118) N-(3-(4-chlorophenyl)-2(S)-(3-cyanophenyl)-1(S)-methylpropyl)-2(S)-benzoyloxy)-3-methylbutanamide,
(119) N-(3-(4-chlorophenyl)-2(S)-(3-cyanophenyl)-1(S)-methylpropyl)-2(R)-benzoyloxy)-3-methylbutanamide,
(120) N-(3-(4-chlorophenyl)-2(S)-(3-cyanophenyl)-1(S)-methylpropyl)-2(S)-benzoyloxy)-phenylacetamide,
(121) N-(3-(4-chlorophenyl)-2(S)-(3-cyanophenyl)-1(S)-methylpropyl)-2 (R)-benzoyloxy)-phenylacetamide,
(122) N-(3-(4-chlorophenyl)-2(S)-(3-cyanophenyl)-1(S)-methylpropyl)-2(R)-(2-(2,2,2-trifluoroethoxy)-benzoyloxy)-butanamide,
(123) N-(3-(4-chlorophenyl)-2(S)-(3-cyanophenyl)-1(S)-methylpropyl)-2(S)-(2,6-dichloro-benzoyloxy)-butanamide,
(124) N-(3-(4-chlorophenyl)-2(S)-(3-cyanophenyl)-1(S)-methylpropyl)-2(R)-(2-ethyl-benzoyloxy)-butanamide,
(125) N-(3-(4-chlorophenyl)-2(S)-(3-cyanophenyl)-1(S)-methylpropyl)-2(R)-(cyclobutane-carbonyloxy)-3,3-dimethyl-butanamide,
(126) N-(3-(4-chlorophenyl)-2(S)-(3-cyanophenyl)-1(S)-methylpropyl)-2 (R)-(cyclobutane-carbonyloxy)-3-phenyl-propanamide,
(127) N-(3-(4-chlorophenyl)-2(S)-(3-cyanophenyl)-1(S)-methylpropyl)-2(R)-(3,3-dimethylbutyryloxy)-3-phenyl-propanamide,
(128) N-(3-(4-chlorophenyl)-2(S)-(3-cyanophenyl)-1(S)-methylpropyl)-2(R)-(2,6-dichlorobenzoyloxy)-3-methylbutanamide,
(129) N-(3-(4-chlorophenyl)-2(S)-(3-cyanophenyl)-1(S)-methylpropyl)-2(S)-(3,3-dimethylbutyryloxy)-2-methyl-3-phenyl-propanamide,
(130) N-(3-(4-chlorophenyl)-2(S)-(3-cyanophenyl)-1(S)-methylpropyl)-2 (R)-(3,3-dimethylbutyryloxy)-2-methyl-3-phenyl-propanamide,
(131) N-(3-(4-chlorophenyl)-2(S)-(3-cyanophenyl)-1(S)-methylpropyl)-2(S)-(cyclobutane-carbonyloxy)-2-methyl-3-phenyl-propanamide, and
(132) N-(3-(4-chlorophenyl)-2(S)-(3-cyanophenyl)-1(S)-methylpropyl)-2 (R)-(cyclobutane-carbonyloxy)-2-methyl-3-phenyl-propanamide,
or a pharmaceutically acceptable salt thereof.

6. The compound according to claim 1, selected from:
(1) N-(3-(4-chlorophenyl)-2(S)-(3-cyanophenyl)-1(S)-methylpropyl)-2-benzoyloxy-2-methyl-propanamide,
(2) N-(3-(4-chlorophenyl)-2(S)-(3-cyanophenyl)-1(S)-methylpropyl)-2-cyclohexylcarbonyloxy-2-methyl-propanamide,
(3) N-(3-(4-chlorophenyl)-2(S)-(3-cyanophenyl)-1(S)-methyl propyl)-2-cyclohexylcarbonyloxy-2-methyl-propanamide,
(4) N-(3-(4-chlorophenyl)-2(S)-(3-cyanophenyl)-1(S)-methylpropyl)-2-cyclopentylcarbonyloxy-2-methyl-propanamide,
(5) N-(3-(4-chlorophenyl)-2(S)-(3-cyanophenyl)-1(S)-methylpropyl)-2-(4-cyano-benzoyloxy)-2-methyl-propanamide,
(6) N-(3-(4-chlorophenyl)-2(S)-(3-cyanophenyl)-1(S)-methylpropyl)-2-(2-trifluoromethoxy-benzoyloxy)-2-methyl-propanamide,
(7) N-(3-(4-chlorophenyl)-2(S)-(3-cyanophenyl)-1(S)-methylpropyl)-2-(2-chloro-benzoyloxy)-2-methyl-propanamide,
(8) N-(3-(4-chlorophenyl)-2(S)-(3-cyanophenyl)-1(S)-methylpropyl)-2-(2-hydroxy-benzoyloxy)-2-methyl-propanamide,
(9) N-(3-(4-chlorophenyl)-2(S)-(3-cyanophenyl)-1(S)-methylpropyl)-2-(2,6-dichloro-benzoyloxy)-2-methyl-propanamide,
(10) N-(3-(4-chlorophenyl)-2(S)-(3-cyanophenyl)-1(S)-methylpropyl)-2-(4-hydroxy-benzoyloxy)-2-methyl-propanamide,
(11) N-(3-(4-chlorophenyl)-2(S)-(3-cyanophenyl)-1(S)-methylpropyl)-2-((2S)-(acetoxy)(phenyl)acetoxy)-2-methyl-propanamide,
(12) N-(3-(4-chlorophenyl)-2(S)-(3-cyanophenyl)-1(S)-methylpropyl)-2-((2R)-(hydroxy)(phenyl)acetoxy)-2-methyl-propanamide,
(13) N-(3-(4-chlorophenyl)-2(S)-(3-cyanophenyl)-1(S)-methylpropyl)-2-phenylacetoxy-2-methyl-propanamide,
(14) N-(3-(4-chlorophenyl)-2(S)-(3-cyanophenyl)-1(S)-methylpropyl)-2-(2-acetoxy-benzoyloxy)-2-methyl-propanamide,
(15) N-(3-(4-chlorophenyl)-2(S)-(3-cyanophenyl)-1(S)-methylpropyl)-2-(2-methyl-benzoyloxy)-2-methyl-propanamide,
(16) N-(3-(4-chlorophenyl)-2(S)-(3-cyanophenyl)-1(S)-methylpropyl)-2-(2,6-dimethyl-benzoyloxy)-2-methyl-propanamide,
(17) N-(3-(4-chlorophenyl)-2(S)-(3-cyanophenyl)-1(S)-methylpropyl)-2-(2-trifluoromethyl-benzoyloxy)-2-methyl-propanamide,
(18) N-(3-(4-chlorophenyl)-2(S)-(3-cyanophenyl)-1(S)-methylpropyl)-2-(3-cyano-benzoyloxy)-2-methyl-propanamide,
(19) N-(3-(4-chlorophenyl)-2(S)-(3-cyanophenyl)-1(S)-methylpropyl)-2-(2-chloro-6-fluoro-benzoyloxy)-2-methyl-propanamide,
(20) N-(3-(4-chlorophenyl)-2(S)-(3-cyanophenyl)-1(S)-methylpropyl)-2-(4-chloro-benzoyloxy)-2-methyl-propanamide,
(21) N-(3-(4-chlorophenyl)-2(S)-(3-cyanophenyl)-1(S)-methylpropyl)-2-(6-chloro-2-fluoro-3-methyl-benzoyloxy)-2-methyl-propanamide,
(22) N-(3-(4-chlorophenyl)-2(S)-(3-cyanophenyl)-1(S)-methylpropyl)-2-(3,3-dimethyl-butyryloxy)-2-methyl-propanamide,
(23) N-(3-(4-chlorophenyl)-2(S)-(3-cyanophenyl)-1(S)-methylpropyl)-2-(3,4-dichloro-benzoyloxy)-2-methyl-propanamide,
(24) N-(3-(4-chlorophenyl)-2(S)-(3-cyanophenyl)-1(S)-methylpropyl)-2-(2-chloro-6-fluoro-3-methyl-benzoyloxy)-2-methyl-propanamide,
(25) N-(3-(4-chlorophenyl)-2(S)-(3-cyanophenyl)-1(S)-methylpropyl)-2-(2-amino-6-fluoro-benzoyloxy)-2-methyl-propanamide,
(26) N-(3-(4-chlorophenyl)-2(S)-(3-cyanophenyl)-1(S)-methylpropyl)-2(S)-(2,6-dichloro-benzoyloxy)-butanamide,
(27) N-(3-(4-chlorophenyl)-2(S)-(3-cyanophenyl)-1(S)-methylpropyl)-2(R)-(2-ethyl-benzoyloxy)-butanamide,
(28) N-(3-(4-chlorophenyl)-2(S)-(3-cyanophenyl)-1(S)-methylpropyl)-2(R)-(2,6-dichlorobenzoyloxy)-3-methylbutanamide, and
(29) N-(3-(4-chlorophenyl)-2(S)-(3-cyanophenyl)-1(S)-methylpropyl)-2(S)-(cyclobutane-carbonyloxy)-2-methyl-3-phenyl-propanamide,
or a pharmaceutically acceptable salt thereof.

7-12. (canceled)

13. A composition comprising a compound according to claim 1 and a pharmaceutically acceptable carrier.

14. A composition comprising a compound according to claim 1 and a compound selected from simvastatin, ezetimibe and sitagliptin, and a pharmaceutically acceptable carrier.

15. A method of treating CB1 receptor mediated diseases by administering to a patient in need of such treatment a therapeutically effective amount of a compound according to claim 1, or a pharmaceutically acceptable salt thereof.

16. The method according to claim 15, wherein the disease mediated by the Cannabinoid-1 receptor is selected from: psychosis, memory deficit, cognitive disorders, Alzheimer's disease, migraine, neuropathy, neuro-inflammatory disorders, cerebral vascular accidents, head trauma, anxiety disorders, stress, epilepsy, Parkinson's disease, schizophrenia, substance abuse disorders, constipation, chronic intestinal pseudo-obstruction, non-alcoholic fatty liver disease, non-alcoholic steatohepatitis, cirrhosis of the liver, asthma, obesity, and other eating disorders associated with excessive food intake.

17. The method according to claim 16, wherein the disease mediated by the Cannabinoid-1 receptor is selected from substance abuse disorders, and eating disorders associated with excessive food intake.

18. The method according to claim 17 wherein the substance abuse disorder is abuse of or addiction to a substance selected from: opiates, alcohol, marijuana, and nicotine, and the eating disorder associated with excessive food intake is selected from obesity, bulimia nervosa, and compulsive eating disorders.

19. The method according to claim 18 wherein the eating disorder associated with excessive food intake is obesity.
